(19) United States
(12) Patent Application Publication
(10) Pub. No.: US 2024/0125795 A1
(43) Pub. Date: Apr. 18, 2024

(54) APPARATUS FOR BIOMOLECULE ASSAY

(71) Applicant: Seer, Inc., Redwood City, CA (US)

(72) Inventors: Daniel Hornburg, Foster City, CA (US); Craig Stolarczyk, San Mateo, CA (US); Behzad Tangeysh, Redwood City, CA (US); Asim Siddiqui, San Francisco, CA (US); Tristan Brown, Sunnyvale, CA (US); Shadi Roshdiferdosi, Belmont, CA (US); Martin Goldberg, Saratoga, CA (US); Moaraj HASAN, San Francisco, CA (US)

(21) Appl. No.: 18/547,831

(22) PCT Filed: Feb. 25, 2022
(86) PCT No.: PCT/US2022/017907

§ 371 Date: Aug. 24, 2023

Related U.S. Application Data
(60) Provisional application No. 63/154,660, filed on Feb. 26, 2021.
(60) Provisional application No. 63/306,951, filed on Feb. 4, 2022.

(51) Int. Cl. G01N 33/68; G01N 33/543

(52) U.S. Cl. CPC G01N 33/6848; G01N 33/54346

(57) ABSTRACT

Disclosed herein are methods for identifying physicochemical properties associated with protein corona formation at the level of proteins and NP-functionalization. Further disclosed herein are compositions comprising combinations of particles configured for low abundance protein collection and deep proteomic analysis.

### CROSS-REFERENCE

The present application claims the benefit of U.S. Provisional Application No. 63/154,660, filed Feb. 26, 2021, and U.S. Provisional Application No. 63/306,951, filed Feb. 4, 2022, each of which is incorporated herein by reference in its entirety.

### BACKGROUND

Analysis of low abundance biomolecules is a major challenge in proteomics. While some recent progress has improved capture for single biomolecules from samples, diagnostic methods often require detection of a large number of biomolecules for accurate biological state, omic, and organismal identifications. Accordingly, compositions and methods are needed for enriching large portions of low abundance biomolecules from samples.

### SUMMARY

In one aspect, provided herein is a method of selecting surfaces for a biomolecule assay, comprising: (a) providing one or more biological samples comprising a plurality of biomolecules; (b) contacting the one or more biological samples with a plurality of surfaces, such that each surface in the plurality of surfaces adsorbs a subset of biomolecules in the plurality of biomolecules; (c) determining, for each surface in the plurality of surfaces, abundances of the subset of biomolecules adsorbed thereon; and (d) selecting a subset of surfaces in the plurality of surfaces based at least in part on the abundances when the subset of surfaces adsorbs biomolecules or biomolecule groups that comprise a different abundance pattern compared to another subset of surfaces in the plurality of surfaces.

In some embodiments, the subset of surfaces is selected when a first surface in the subset of surfaces binds a first set of functionally-related and/or structurally-related biomolecules. In some embodiments, the subset of surfaces is selected when a second surface in the subset of surfaces binds a second set of functionally-related and/or structurally-related biomolecules.

In some embodiments, the first set, the second set, or both functionally related biomolecules comprises at least one of: a hormonal protein, a cytolytic protein, an innate immunity protein, a membrane attack complex, a complement pathway protein, an amyloid fibril, a protein involved in cholesterol metabolism, a protein involved in steroid metabolism, a protein with gamma carboxyglutamic acid domains, a protein associated with amyloidosis, a sulfated protein, a proteoglycan protein, an immunoglobulin, an adaptive immunity protein, a mitochondrial protein, a membrane protein, a cell shape protein, a muscular protein, a protein that binds to genetic material, a protein associated with gene expression and/or regulation, a protein associated with intra and/or extracellular space, and any combination thereof.

In some embodiments, the method may further comprise contacting a new biological sample, not among the one or more biological samples, with the subset of surfaces to thereby assay the first set or the second set of functionally related and/or structurally-related biomolecules in the new biological sample.

In some embodiments, the first surface and the second surface each adsorbs a given biomolecule in the plurality of biomolecules at a different relative abundance. In some embodiments, the first surface adsorbs at least one biomolecule that is not adsorbed on the second surface.

In some embodiments, the one or more biological samples are samples obtained from subjects afflicted with a given disease, such that the selected subset of surfaces is optimized for assaying a new biological sample for the given disease.

In some embodiments, the method further comprises contacting a new biological sample, not among the one or more biological samples, with the subset of surfaces to thereby probe biomolecules in the new biological sample for determining a disease state of the new biological sample related to the given disease.

In some embodiments, the one or more biological samples are obtained from an individual, such that the selected subset of surfaces is optimized for assaying biological samples from the individual. In some embodiments, the one or more biological samples are obtained from a group of individuals having at least one attribute, such that the selected subset of surfaces is optimized for assaying biological samples from individuals having the at least one attribute. In some embodiments, the at least one attribute comprises a genetic factor, a non-genetic factor, or both. In some embodiments, the genetic factor comprises one or more genetic mutations, presence or absence of one or more alleles, presence or absence of one or more genes, presence or absence of one or more chromosomes, or any combination thereof. In some embodiments, the non-genetic factor comprises a level of physical activity, quality and pattern of sleep, consumption of drugs and/or alcohol, biometrics, or any combination thereof. In some embodiments, the one or more biological samples are samples obtained from one or more species, such that the selected subset of surfaces is optimized for assaying for the at least one species in the one or more species.

In some embodiments, the first surface and the second surface is chosen when a Jaccard index between the identities of the distinct subset of biomolecules adsorbed on the first surface and the second surface is at most about 0.1, 0.2, 0.3, 0.4, 0.5, 0.6, 0.7, 0.8, 0.9. In some embodiments, the first surface and the second surface is chosen when a Pearson correlation index between measured intensities of the first set of functionally-related biomolecules and the second set of functionally-related biomolecules is at most about 0.1, 0.2, 0.3, 0.4, 0.5, 0.6, 0.7, 0.8, 0.9, or 1.

In some embodiments, the subset of surfaces is selected when the subset of surfaces adsorbs biomolecule or biomolecule groups at a greater dynamic range compared to another subset of surfaces in the plurality of surfaces. In some embodiments, the greater dynamic range is at least about 1, 2, 3, 4, 5, 6, 7, 8, 9, or 10 magnitudes greater.

In some embodiments, the one or more biological samples comprises derivatives or portions of the same given biological sample. In some embodiments, the one or more biological samples comprises human blood plasma samples. In some embodiments, the one or more biological samples comprises a biological sample standard. In some embodiments, the biological sample standard is a HeLa cell extract.

In some embodiments, the plurality of biomolecules comprises polyamino acids. In some embodiments, the polyamino acids comprise peptides, proteins, or a combination thereof.

In some embodiments, the distinct subset of biomolecules adsorbed on at least one surface in the plurality of surfaces comprises at least two biomolecules that do not share a common binding motif.

In some embodiments, the determining the identities in (c) is performed by: (i) desorbing the distinct subset of biomolecules adsorbed on each surface in the plurality of surfaces to produce desorbed biomolecules, (ii) performing mass spectrometry on the desorbed biomolecules to produce mass spectrometry signals, and (iii) quantifying the mass spectrometry signals to determine the identities of the distinct subset of biomolecules. In some embodiments, (i) further comprises digesting at least a portion of the distinct subset of biomolecules to produce desorbed biomolecules. In some embodiments, the digesting comprises contacting the distinct subset of biomolecules with a protease.

In some embodiments, each surface in the plurality of surfaces adsorbs a distinct subset of biomolecules in the plurality of biomolecules. In some embodiments, a first distinct subset of biomolecules adsorbed on a first surface in the plurality of surfaces comprises at least one common biomolecule with a second subset of biomolecules adsorbed on a second surface in the plurality of surfaces. In some embodiments, the first distinct subset of biomolecules and the second subset of biomolecules comprises at least one biomolecule not in common.

In some embodiments, the different abundance pattern comprises enrichment of low abundance biomolecules relative to the plurality of biomolecules in the one or more biological samples.

In another aspect, described herein is a method of producing an enriched biological sample, comprising: (a) providing a sample comprising a plurality of biomolecules; (b) contacting the sample with a particle or resin to specifically bind at least one biomolecule or biomolecule class target in the sample to the particle or resin; (c) separating the particle or resin and the at least one biomolecule from the sample, thereby producing a depleted sample; (d) contacting the depleted sample with a surface, wherein the surface is configured to adsorb a set of biomolecules in the depleted sample on the surface; (e) separating the set of biomolecules and the surface from the depleted sample; and (f) releasing the set of biomolecules from the surface to produce an enriched sample comprising the set of biomolecules.

In some embodiments, the at least one biomolecule or biomolecule class target comprises: albumin, IgG, IgA, IgM, IgD, IgE, IgG (light chain), alpha-1-acid glycoprotein, fibrinogen, haptoglobin, alpha-1-antitrypsin, alpha-2-macroglobulin, transferrin, apolipoprotein A-1, or any combination thereof.

In some embodiments, at least one biomolecule or biomolecule class target comprises a predetermined subset of the plurality of biomolecules comprising a high relative abundance.

In some embodiments, in step (c), the separating reduces an abundance of the at least one biomolecule or biomolecule class target at least by a factor of 2, 5, 10, or 100. In some embodiments, in step (c), producing the depleted sample yields at least about 30% more unique proteins, protein groups, or peptides in the enriched sample of step (f). In some embodiments, in step (c), producing the depleted sample yields a larger dynamic range of at least about 1 magnitude in the unique proteins or protein groups in the enriched sample of step (f).

In some embodiments, the method may further comprise after step (c) or before step (d), drying and reconstituting the depleted sample to a predetermined concentration or volume.

In some embodiments, the method may further comprise after step (e), drying and reconstituting the enriched sample to a predetermined concentration or volume.

In some embodiments, the method is performed in less than about 72 hours.

In some embodiments, the biomolecule comprises a protein or protein group.

In some embodiments, the surface is a nanoparticle surface.

In some embodiments, the method may further comprise contacting the depleted sample with a second surface, wherein the second surface is configured to adsorb a second set of biomolecules in the depleted sample on the second surface.

In some embodiments, the releasing in (f) further comprises digesting the set of biomolecules.

In some embodiments, the particle or resin is disposed in a column.

In another aspect, described herein is a kit for enriching a biological sample, comprising: (a) a first substance configured to specifically bind to a first set of biomolecule targets; (b) a second substance configured to adsorb a second set of biomolecule targets; and (c) a third substance configured to adsorb a third set of biomolecule targets.

In some embodiments, the first substance is a resin or a particle. In some embodiments, the first substance comprises a specific binding moiety configured to bind to the first set of biomolecule targets. In some embodiments, the first substance is configured to specifically bind to at least one of: albumin, IgG, IgA, IgM, IgD, IgE, IgG (light chains), alpha-1-acid glycoprotein, fibrinogen, haptoglobin, alpha-1-antitrypsin, alpha-2-macroglobulin, transferrin, and apolipoprotein A-1.

In some embodiments, the kit may further comprise a fourth substance configured to non-specifically bind to a fourth set of biomolecule targets.

In some embodiments, the kit may further comprise a fifth substance configured to non-specifically bind to a fifth set of biomolecule targets.

In some embodiments, the second substance comprises a plurality of domains, wherein each domain in the plurality of domains is configured to non-specifically bind to a distinct subset in the second set of biomolecule targets. In some embodiments, the second substance comprises a particle surface, and the plurality of domains comprises a plurality of surface regions on the particle surface. In some embodiments, the second substance comprises a plurality of particle surfaces, and the plurality of particle surfaces are disposed on a plurality of particles.

In some embodiments, the kit comprises a chamber or a well having the first substance, the second substance, and the third substance disposed therein. In some embodiments, the chamber comprises a column. In some embodiments, the chamber comprises a microfluidic channel.

In some embodiments, a surface region of the well comprises the first substance.

Various aspects of the present disclosure provide methods for determining particle properties, and further for identifying combinations of particle properties which enable deep omic profiling of biological samples. The present disclosure further provides compositions comprising particles with combinations of physicochemical properties optimized for defined capture of proteins (e.g., tailored to a subset of proteins, protein classes, or broad capture of proteins across multiple classes) from biological samples.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

The patent or application file contains at least one drawing executed in color. Copies of this patent or patent application publication with color drawing(s) will be provided by the Office upon request and payment of the necessary fee.

FIG. 1 shows a schematic of a workflow for particle-based protein collection from a biological sample for mass spectrometric analysis.

FIG. 2 depicts example types of surface functionalization for particles, in accordance with some embodiments.

FIG. 3A shows a plot of mass spectrometric intensities for different proteins collected from plasma on 37 different particles, in accordance with some embodiments.

FIG. 3B shows a hierarchical clustering performed on 37 particles with different combinations of physicochemical properties based on protein corona compositions of functionally and/or structurally related proteins formed with each particle, in accordance with some embodiments.

FIG. 3C shows a hierarchical clustering performed on particles with different combinations of physicochemical properties based on protein corona compositions of structurally related proteins formed with each particle, in accordance with some embodiments.

FIG. 4A shows a plot of variance decomposition performed on the protein corona data shown in FIGS. 3A-3B, illustrating the degree to which different factors may contribute to observed protein intensities, in accordance with some embodiments.

FIG. 4B shows a plot of degrees of variance in protein intensities as functions of different particle properties for the 37 particles shown in FIGS. 3A-B and FIG. 4A, in accordance with some embodiments.

FIG. 5A shows a schematic of four different workflows for plasma protein purification and analysis, in accordance with some embodiments. Some workflows may utilize particles, plasma depletion methods, neat plasma, denaturation, reduction/alkylation, protein digestion, magnetic separation, peptide fractionation, high-pH fractionation, or any combination thereof.

FIG. 5B shows the number of protein groups identified with workflows shown in FIG. 5A, in accordance with some embodiments.

FIG. 5C shows variations in peptide intensities over multiple LC-MS/MS parameters, including LC-gradient length and MS instrumentation, in accordance with some embodiments.

FIG. 5D shows human plasma abundances of proteins identified with each workflow shown in FIG. 5A, in accordance with some embodiments.

FIG. 5E shows proteomic data including plasma proteome coverage from each workflow shown in FIG. 5A, in accordance with some embodiments.

FIG. 5F shows the overlap between the identified protein groups across each of the workflows shown in FIG. 5A, in accordance with some embodiments.

FIG. 5G shows the number of protein group identifications for the five particle panel and the high-pH depletion workflows shown in FIG. 5A for 7 different protein annotations, in accordance with some embodiments.

FIG. 6A shows the median number of protein groups identified for each workflow shown in FIG. 5A utilizing data-dependent mass spectrometric acquisition, in accordance with some embodiments.

FIG. 6B shows coefficients of variation (CV) of median normalized peptide intensities filtered for data-dependent mass spectrometric identifications across the five particle panel, high-pH fractionation (“Deep Fractionation”), and neat plasma workflows shown in FIG. 5A, in accordance with some embodiments.

FIG. 6C shows the dynamic range of proteins identified with each workflow shown in FIG. 6A-6B, in accordance with some embodiments.

FIG. 6D shows percent coverage of the human proteome in some workflows (top) and a comparison of the relative coverage of the human proteome by the five particle panel workflow and the first high-pH fractionation sample (bottom) of FIGS. 6A-C, in accordance with some embodiments.

FIG. 7 shows the median number of protein groups identified, the Jaccard index, correlation coefficient, and coefficient of variation from 10 particles as well as neat and depleted plasma, in accordance with some embodiments.

FIG. 8 shows clustering analysis performed on the protein intensity and overlap results shown in FIG. 7 as a distance tree for the particles, depleted plasma, and neat plasma, in accordance with some embodiments.

FIG. 9 shows TEM images of each particle, along with their zeta potential, hydrodynamic radii, and polydispersity index (PDI, bar graphs below images), in accordance with some embodiments.

FIG. 10A shows a volcano plot depicting the coefficients derived from a model for protein binding based on the properties of three specific particles, in accordance with some embodiments.

FIG. 10B shows results from 500 random samplings selecting 2×12 non-overlapping subjects assayed with the 10 particles, in accordance with some embodiments.

FIG. 10C shows correlation coefficients between the determined coefficients for each protein and the zeta potential of the particle, in accordance with some embodiments.

FIG. 11 shows a computer system that is programmed or otherwise configured to implement methods provided herein, in accordance with some embodiments.

FIG. 12 shows the number of proteins collected on and subsequently identified by mass spectrometry following collection on particle panels comprising from 1 to 12 particles, in accordance with some embodiments.

FIG. 13A provides an example workflow for enriching subsets of biomolecules from a biological sample, in accordance with some embodiments.

FIG. 13B provides an example workflow for assaying biomolecules from a biological sample, in accordance with some embodiments.

FIG. 14 shows time used for various plasma protein assay steps for a particle-based method and for two high-pH depletion based methods, in accordance with some embodiments.

FIG. 15 shows an apparatus, in accordance with some embodiments.

FIG. 16 shows transfer units, in accordance with some embodiments.

FIG. 17 shows an apparatus and components thereof, in accordance with some embodiments.

FIG. 18 shows an apparatus and components thereof, in accordance with some embodiments.

FIG. 19 shows a transfer unit, in accordance with some embodiments.

FIG. 20 shows a transfer unit, in accordance with some embodiments.

FIG. 21 shows an illustration of a plurality of partitions, in accordance with some embodiments.

FIG. 22 shows transfer units, in accordance with some embodiments.

FIG. 23 shows an illustration of an apparatus and components thereof, in accordance with some embodiments.

FIG. 24 shows a schematic of a workflow for assaying biomolecules from a biological sample using a trap column, in accordance with some embodiments.

FIG. 25 shows a schematic of a workflow for assaying biomolecules from a biological sample using a trap column, in accordance with some embodiments.

FIG. 26 shows a schematic of a workflow for assaying biomolecules from a biological sample using nanoparticles and a resin or bead, in accordance with some embodiments.

FIG. 27 shows a schematic of a workflow for assaying biomolecules from a biological sample using a depletion step, in accordance with some embodiments.

FIG. 28 shows biomolecules for removal by a depletion column, in accordance with some embodiments.

FIGS. 29A-29B shows a depletion column and a depletion well, respectively, in accordance with some embodiments.

FIG. 30 shows protein yields from depletion experiments and control experiments, in accordance with some embodiments.

FIG. 31 shows coefficient of variance from depletion experiments and control experiments, in accordance with some embodiments.

FIG. 32 shows protein group identifications from depletion experiments and control experiments, in accordance with some embodiments.

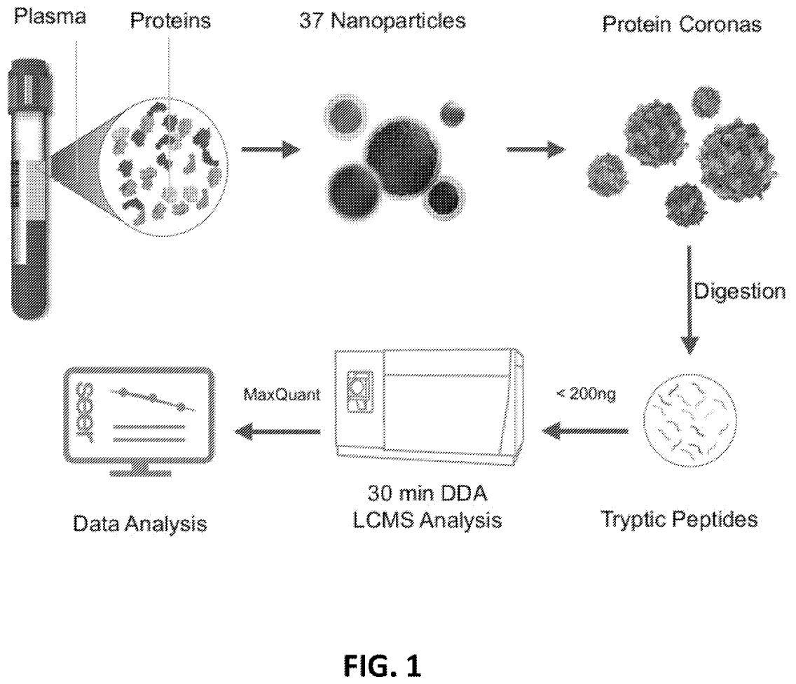

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed. Further, headings provided herein are for convenience only and do not interpret the scope or meaning of the claimed disclosure. The section headings used herein are for organizational purposes only and are not to be construed as limiting the subject matter described.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The term “optional” or “optionally” denotes that a subsequently described event or circumstance can but need not occur, and that the description includes instances where the event or circumstance occurs and instances in which it does not.

The term “about” means within ±1 of the last significant digit of a given value. For example, if it is stated, “the depleted sample yields at least about 30% more unique proteins, protein groups, or peptides in the enriched sample”, it is implied that the yield is between 20% to 40%. In another example, if it is stated, “the depleted sample yields at least about 35% more unique proteins, protein groups, or peptides in the enriched sample”, it is implied that the yield is between 34% to 36%.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All references cited herein are incorporated by reference in their entirety as though fully set forth.

Biological samples can be complex mixtures which can contain vast arrays of biomolecules with disparate properties. The presence or absence and concentration of various biomolecules, as well as correlations between various subsets of biomolecules (e.g., proteins and nucleic acids), may be indicative of the biological state of a sample (e.g., a healthy or a disease state). For example, a pattern of abundances of dilute plasma proteins may be strongly indicative of the health status of a human subject. However, some enrichment methods may fail to capture more than a narrow subset of biomolecules.

Disclosed herein are systems, methods, and compositions for assaying biomolecules in a biological sample using one or more surfaces that are configured to capture a broad subset of the biomolecules. In some cases, this may be achieved by using one or more surfaces that are configured to adsorb numerous distinct types of biomolecules thereon. Biomolecules assayed in this way may provide a finer resolution of the composition of the biological sample, which may then lead to greater clarity and insights into the biology or contents of a biological sample.

Also described herein are methods for selecting one or more surfaces for a predetermined function. While there may be various surfaces having various physicochemical properties at disposal for assaying biomolecules, one, or a particular combination of the surfaces, may provide an advantageous assay for a predetermined function. For example, the one or more surfaces may be selected for detecting a particular disease, which may be useful for both diagnosis and prognosis. In some cases, the one or more surfaces may be selected for obtaining or monitoring an individual's biological state over time. Still in other cases, the one or more surfaces may be selected for detecting a specific group of biomolecules associated with a given biochemical system (e.g., hormones). One or more surfaces may be selected for various predetermined functions which is described in further detail herein.

Methods for Selecting Particles and/or Surfaces

In some aspects, the present disclosure describes a method of selecting surfaces for a biomolecule assay. In some cases, the method comprises providing one or more biological samples comprising a plurality of biomolecules. In some cases, the method comprises contacting the one or more biological samples with a plurality of surfaces. In some cases, each surface in the plurality of surfaces adsorbs a subset of biomolecules in the plurality of biomolecules. In some cases, the method comprises determining, for each surface in the plurality of surfaces, abundances of the subset of biomolecules adsorbed thereon. In some cases, the method comprises determining, for at least a subset of the surfaces in the plurality of surfaces, abundances of the subset of biomolecules adsorbed thereon. In some cases, the method comprises selecting a subset of surfaces in the plurality of surfaces based at least in part on the abundances when the subset of surfaces adsorbs biomolecules or biomolecule groups that comprise a different abundance pattern compared to another subset of surfaces in the plurality of surfaces.

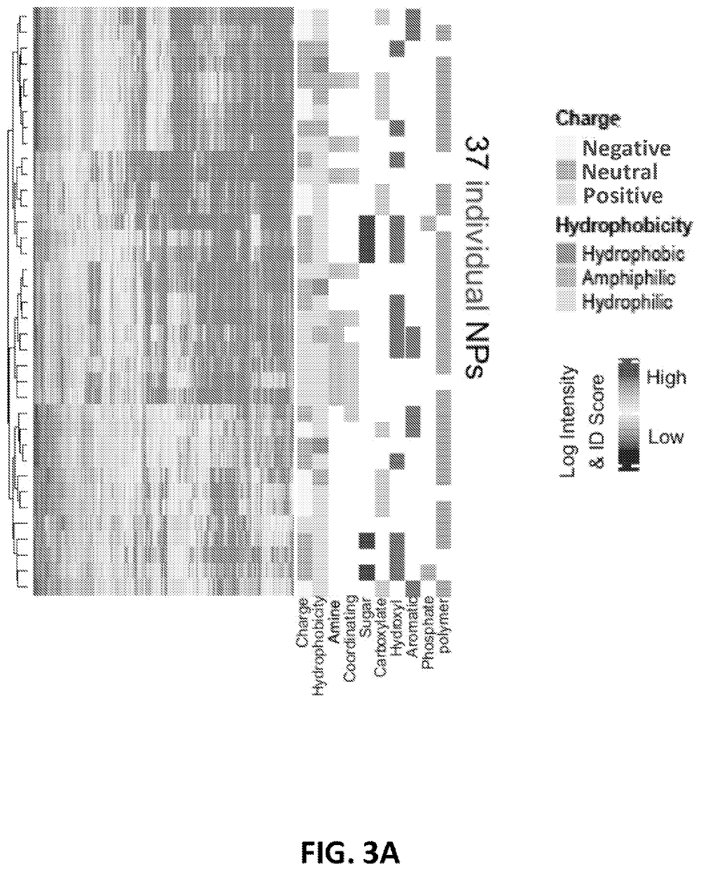

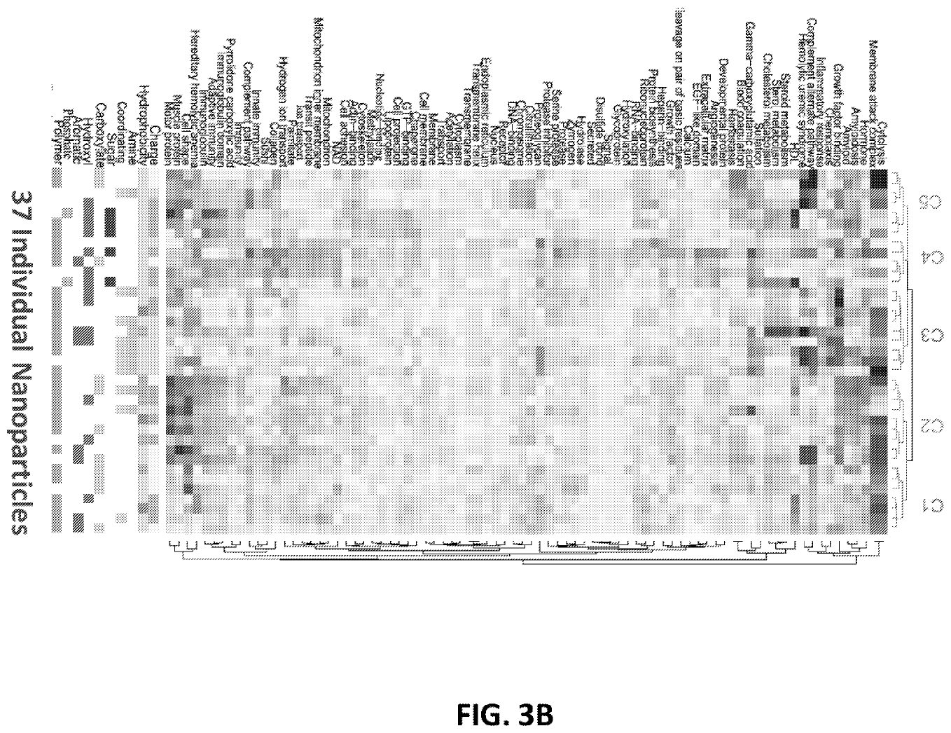

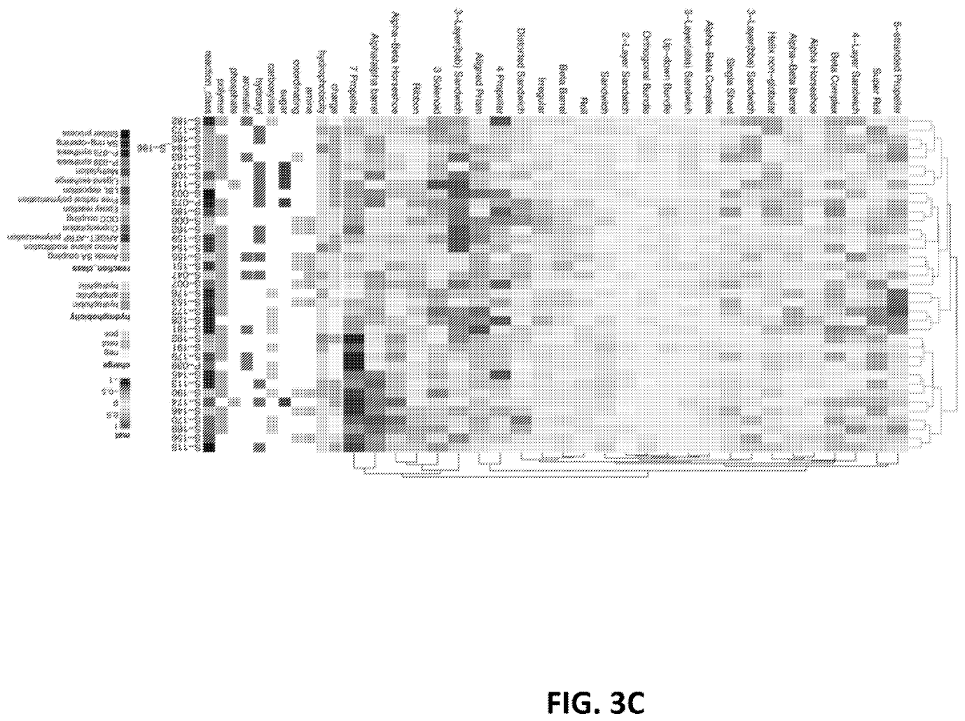

In some cases, the subset of surfaces is selected when a first surface in the subset of surfaces binds a first set of functionally-related biomolecules. In some cases, the subset of surfaces is selected when a second surface in the subset of surfaces binds a second set of functionally-related biomolecules of the same sample. FIG. 3A illustrates an example of hierarchical clustering of particles and based on protein intensities that were measured using the particles. Each particle exhibited a characteristic pattern of biomolecules that were adsorbed and subsequently measured. FIG. 3B illustrates an example of 1D annotation enrichment scores for biomolecules based on Uniprot Keywords, which show that some particles adsorbed certain functionally-related sets of biomolecules.

In some cases, the subset of surfaces is selected when a first surface in the subset of surfaces binds a first set of structurally-related biomolecules. In some cases, the subset of surfaces is selected when a second surface in the subset of surfaces binds a second set of structurally-related biomolecules of the same sample. FIG. 3C illustrates an example of 1D annotation enrichment scores for biomolecules based on Uniprot Keywords, which show that some particles adsorbed certain structurally-related sets of biomolecules based on Class, Architecture, Topology, Homology (CATH) annotation. For example, some CATH architectures representing the secondary structures of proteins are enriched as a function of the charge of the NP surface functionalization. Enriched annotations are indicated in red, while depleted annotations are indicated in blue.

The subset of surfaces may be selected such that the subset is specialized for assaying proteins associated with motor function (e.g., Uniprot keywords for muscle protein, motor protein, cell shape, etc.). In some cases, the subset of surfaces may be selected such that the subset is specialized for assaying proteins associated with metabolism of sterols and related molecules (e.g., Uniprot keywords for steroid metabolism, sterol metabolism, cholesterol metabolism, etc.). In some cases, the subset of surfaces may be selected such that the subset is specialized for assaying as broadly as possible the various functionally related biomolecules.

In some cases, the first set, the second set, or both functionally related biomolecules may comprise at least one of: a hormonal protein, a cytolytic protein, an innate immunity protein, a membrane attack complex, a complement pathway protein, an amyloid fibril, a protein involved in cholesterol metabolism, a protein involved in steroid metabolism, a protein with gamma carboxyglutamic acid domains, a protein associated with amyloidosis, a sulfated protein, a proteoglycan protein, an immunoglobulin, an adaptive immunity protein, a mitochondrial protein, a membrane protein, a cell shape protein, a muscular protein, a protein that binds to genetic material, a protein associated with gene expression and/or regulation, a protein associated with intra and/or extracellular space, and any combination thereof. Any one or combination thereof functionally related biomolecules may be of interest, and the subset of surfaces may be selected to target those biomolecules.

A selected subset of surfaces may be applied to new samples. In some cases, the method may comprise contacting a new (e.g., separate) biological sample, not among the one or more biological samples, with the subset of surfaces to thereby assay the first set or the second set of functionally related biomolecules in the new biological sample. In some cases, the method comprises contacting a new biological sample, not among the one or more biological samples, with the subset of surfaces to thereby probe biomolecules in the new biological sample for determining a disease state of the new biological sample related to the given disease. In some cases, a subset of surfaces or particles may be selected to assay specifically any one of the diseases disclosed herein.

In some cases, the one or more biological samples are samples obtained from subjects afflicted with a given disease, such that the selected subset of surfaces is optimized for assaying a new biological sample for the given disease. Therefore, the subset may be used to assay a new biological sample from a subject to determine if the subject has the given disease.

In some cases, the one or more biological samples are obtained from an individual, such that the selected subset of surfaces is optimized for assaying biological samples from the individual. In some cases, the subset of surfaces may be selected based on an ability to broadly assay (e.g., assaying numerous unique proteins or protein groups) a specific biological sample from one individual (e.g., blood plasma from a person). The subset of surfaces may then be used to monitor the individual's biological state over time. In some cases, the one or more biological samples comprises derivatives or portions of the same given biological sample.

In some cases, the one or more biological samples are obtained from a group of individuals having at least one attribute, such that the selected subset of surfaces is optimized for assaying biological samples from individuals having the at least one attribute. In some cases, the at least one attribute may comprise a genetic factor, a non-genetic factor, or both. In some cases, the one or more biological samples are obtained from a group of individuals having at least two attributes, such that the selected subset of surfaces is optimized for assaying biological samples from individuals having the at least two attributes. In some cases, the at least two attributes may comprise at least a genetic factor, a non-genetic factor, or both.

For example, the subset of surfaces may be optimized to assay biomolecules for individuals having a given genetic disease. In another example, the subset of surfaces may be optimized to assay biomolecules for individuals from a certain geographic region, thereby having a similarity in genetics. In some cases, a genetic factor may comprise one or more genetic mutations, presence or absence of one or more alleles, presence or absence of one or more genes, presence or absence of one or more chromosomes, or any combination thereof.

In some cases, the non-genetic factor comprises a level of physical activity, quality and pattern of sleep, consumption of drugs and/or alcohol, biometrics, a certain lifestyle, a socioeconomic status, geographic area of residence, exposure to certain pollutants, a quantitative metric thereof, or any combination thereof. In some cases, a quantitative metric may be a self-reported by an individual (e.g., responses to a questionnaire or a survey), an assessment by a healthcare professional, an assessment by an individual collecting information and/or data, or a combination thereof.

In some cases, the one or more biological samples are samples obtained from one or more species, such that the selected subset of surfaces is optimized for assaying for the at least one species in the one or more species.

The subset of surfaces may be selected to be specialized for various types of biological samples. In some cases, a biological sample may be a human blood plasma sample, saliva, feces, tears, a cell, a tissue, an organ, or any other biological sample disclosed herein. In some cases, a biological sample may be a portion of human blood plasma, feces, tears, a cell, a tissue, an organ, or any other biological sample disclosed herein.

Performance of a given subset of surfaces may be assessed using a biological sample standard. In some examples, the biological sample standard may be a HeLa cell extract. In some examples, the biological sample standard may be a spiked protein (e.g., E. coli). In some examples, the biological sample standard may be non-homologous to the species under study.

Differences in the adsorption of biomolecules between surfaces in the subset may be characterized in various ways. In some cases, the first surface and the second surface may adsorb similar biomolecules from a biological sample. In some cases, the first surface adsorbs at least one biomolecule that is not adsorbed on the second surface. In some cases, the second surface adsorbs at least one biomolecule that is not adsorbed on the first surface. In some cases, the relative abundances of the biomolecules adsorbed may be different between the first surface and the second surface. This may be useful when assaying the adsorbed biomolecules using an assay that may have stochasticity (e.g., some cases of mass spectrometry), where higher abundances of a given biomolecule may provide higher probability of detecting the given biomolecule. Therefore, in some cases, the first surface and the second surface may each adsorb a given biomolecule in the plurality of biomolecules at a different relative abundance. In some cases, the relative abundances of the biomolecules adsorbed may be the same between the first surface and the second surface.

In some cases, the first surface and the second surface are chosen when a Jaccard index between the identities of the distinct subset of biomolecules adsorbed on the first surface and the second surface is at most about 0.1, 0.2, 0.3, 0.4, 0.5, 0.6, 0.7, 0.8, or 0.9. In some cases, the first surface and the second surface are chosen when a Pearson correlation index between measured intensities of the first set of functionally-related biomolecules and the second set of functionally-related biomolecules is at most about 0.1, 0.2, 0.3, 0.4, 0.5, 0.6, 0.7, 0.8, 0.9, or 1.0. In some cases, the identities and/or the intensities may be determined by mass spectrometry performed on adsorbed biomolecules on the first surface and the second surface. For example, the determining the identities or the intensities may be performed by: (i) desorbing the distinct subset of biomolecules adsorbed on each surface in the plurality of surfaces to produce desorbed biomolecules, (ii) performing mass spectrometry on the desorbed biomolecules to produce mass spectrometry signals, and (iii) quantifying the mass spectrometry signals to determine the identities and/or intensities of the distinct subset of biomolecules.

In some cases, the subset of surfaces is selected when the subset of surfaces adsorbs biomolecule or biomolecule groups at a greater dynamic range compared to another subset of surfaces in the plurality of surfaces. In some cases, the greater dynamic range is at least about 1, 2, 3, 4, 5, 6, 7, 8, 9, 10, or more magnitudes greater.

In some cases, the distinct subset of biomolecules adsorbed on at least one surface in the plurality of surfaces comprises at least two biomolecules that do not share a common binding motif. In some cases, (i) further comprises digesting at least a portion of the distinct subset of biomolecules to produce desorbed biomolecules. In some cases, the digesting comprises contacting the distinct subset of biomolecules with a protease. In some cases, each surface in the plurality of surfaces adsorbs a distinct subset of biomolecules in the plurality of biomolecules. In some cases, a first distinct subset of biomolecules adsorbed on a first surface in the plurality of surfaces comprises at least one common biomolecule with a second subset of biomolecules adsorbed on a second surface in the plurality of surfaces. In some cases, the first distinct subset of biomolecules and the second subset of biomolecules comprises at least one biomolecule not in common. In some cases, the different abundance pattern comprises enrichment of low abundance biomolecules relative to the plurality of biomolecules in the one or more biological samples.

### Depletion Enhanced Proteomics

In some aspects, the present disclosure describes a method of producing an enriched biological sample. In some aspects, a biomolecule of interest (e.g., a low abundance protein) may be enriched in a biomolecule corona relative to the untreated sample (e.g., a sample that is not assayed using particles). The biomolecule of interest may be a protein. The biomolecule corona may be a protein corona. A level of enrichment may be the percent increase or fold increase in relative abundance of the biomolecule of interest (e.g., number of copies of the biomolecule of interest versus the total number of biomolecules) in the biomolecule corona as compared to the biological sample from which the biomolecule corona was collected. A biomolecule of interest may be enriched in a biomolecule corona by increasing the abundance of the biomolecule of interest in the biomolecule corona as compared to the sample that has not been contacted to the sensor element. A biomolecule of interest may be enriched by decreasing the abundance of a biomolecule that is in high abundance biological sample.

In some cases, a biomolecule or biomolecule class target in a biological sample may be depleted. In some cases, the depletion may allow for the detection of more unique proteins or protein groups in an assay, for example, an assay that comprises contacting the depleted biological sample with a non-specific binding surface is disclosed herein, and performing mass spectrometry on biomolecules adsorbed on the non-specific binding surface. In some cases, depletion may refer to a reduction in abundance of a given biomolecule by factor of at least about 2, 5, 10, 100, or more.

In some cases, the method comprises providing a sample comprising a plurality of biomolecules. In some cases, the method comprises contacting the sample with a particle or functional resin to specifically bind at least one biomolecule or biomolecule class target in the sample to the particle or resin. In some cases, the method comprises separating the particle or resin and then at least one biomolecule from the sample, thereby producing a depleted sample. In some cases, the method comprises contacting the depleted sample with a surface, wherein the surface is configured to adsorb a set of biomolecules remaining in the depleted sample on the surface. In some cases, the method comprises separating the set of biomolecules and the surface from the depleted sample. In some cases, the method comprises releasing the set of biomolecules from the surface to produce an enriched sample comprising the set of biomolecules. In some cases, the method may comprise drying and reconstituting the depleted sample to a predetermined concentration or volume. In some cases, the particle or resin may be disposed in a column.

A biomolecule or biomolecule class target may be any biomolecule in a biological sample. In some cases, the biomolecule or biomolecule class target may comprise a high abundance (e.g., relative to other biomolecules or biomolecule classes) in the biological sample. In some cases, the a biomolecule or biomolecule class target may comprise: albumin, IgG, IgA, IgM, IgD, IgE, IgG (light chain), alpha-1-acid glycoprotein, fibrinogen, haptoglobin, alpha-1-antitrypsin, alpha-2-macroglobulin, transferrin, apolipoprotein A-1, or any combination thereof. These biomolecules may be high abundance biomolecules in human blood plasma samples. However, it shall be understood that other biomolecules or biomolecule classes may be targeted for depletion, since there are many varieties of biological samples, and various biological samples may have different high abundance biomolecules.

In some cases, producing the depleted sample may yield at least about 10%, 20%, 30%, 40%, 50%, 60%, 70%, 80%, 90%, or 100% more unique biomolecules, biomolecule classes, proteins, protein groups, or peptides in the enriched sample that are detected, for example, using mass spectrometry.

In some cases, producing the depleted sample yields a larger dynamic range of at least about 1, 2, 3, 4, 5, 6, 7, 8, 9, 10, or more magnitudes in the unique biomolecules or biomolecule classes, unique proteins or protein groups in the enriched sample that are detected, for example, using mass spectrometry.

In some cases, the method is performed in less than about a week, 6 days, 5 days, 4 days, 3 days, 2 days, 1 day, 12 hours, 6 hours, 4 hours, 2 hours, 1 hour, or less.

Properties and Types of Particles and/or Surfaces

Particle types and surface types consistent with the methods disclosed herein can be made from various materials. As used herein, a “surface” may refer to a surface of a particle. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, glass, ceramics, metal-organic frameworks (MOF), polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron and cadmium, or any other material described in U.S. Pat. No. 7,749,299. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material).

A particle or surface may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer that is polymerized in-situ or coupled to the particle as a polymer), or any combination thereof. Examples of polymers include any one of or any combination of polyethylenes, polycarbonates, polyanhydrides, polyimides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated, or may comprise a degree of phase separation. The polymer may comprise a lipid-terminated polyalkylene glycol and a polyester, or any other material disclosed in U.S. Pat. No. 9,549,901.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol, or any other material listed in U.S. Pat. No. 9,445,994, which is incorporated herein by reference in its entirety.

Examples of particles of the present disclosure are provided in TABLE 2.

TABLE 2
Example particles of the present disclosure

| Batch No. | Type | Particle ID | Description |
|---|---|---|---|
| S-001-001 | HX-13 | SP-001 | Carboxylate (Citrate) superparamagnetic iron oxide NPs |
| | | | (SPION) |
| S-002-001 | HX-19 | SP-002 | Phenol-formaldehyde coated SPION |
| S-003-001 | HX-20 | SP-003 | Silica-coated superparamagnetic iron oxide NPs |
| | | | (SPION) |
| S-004-001 | HX-31 | SP-004 | Polystyrene coated SPION |
| S-005-001 | HX-38 | SP-005 | Carboxylated Poly(styrene-co-methacrylic acid), P(St- |
| | | | co-MAA) coated SPION |
| S-006-001 | HX-42 | SP-006 | N-(3-Trimethoxysilylpropyl)diethylenetriamine coated |
| | | | SPION |
| S-007-001 | HX-56 | SP-007 | poly(N-(3-(dimethylamino)propyl) methacrylamide) |
| | | | (PDMAPMA)-coated SPION |
| S-008-001 | HX-57 | SP-008 | 1,2,4,5-Benzenetetracarboxylic acid coated SPION |
| S-009-001 | HX-58 | SP-009 | PVBTMAC coated |
| | | | poly(vinylbenzyltrimethylammonium chloride) |
| | | | (PVBTMAC) coated SPION |
| S-010-001 | HX-59 | SP-010 | Carboxylate, PAA coated SPION |
| S-011-001 | HX-86 | SP-011 | poly(oligo(ethylene glycol) methyl ether methacrylate) |
| | | | (POEGMA)-coated SPION |
| S-163-001 | | SP-163 | Cis-ubiquitin-functionalized styrene particle |
| S-164-001 | | SP-164 | Ubiquitin-functionalized styrene particle |
| P-033-001 | P33 | SP-333 | Carboxylate functionalized 1 μm magnetic |
| | | | microparticle, surfactant free SPION |
| P-039-003 | P39 | SP-339 | Polystyrene carboxyl functionalized SPION |
| P-041-001 | P41 | SP-341 | Carboxylic acid SPION |
| P-047-001 | P47 | SP-365 | Silica SPION |
| P-048-001 | P48 | SP-348 | Carboxylic acid, 150 nm SPION |
| P-053-001 | P53 | SP-353 | Amino surface microparticle, 0.4-0.6 μm SPION |
| P-056-001 | P56 | SP-356 | Silica amino functionalized microparticle, 0.1-0.39 μm |
| | | | SPION |
| P-063-001 | P63 | SP-363 | Jeffamine surface, 0.1-0.39 μm SPION |
| P-064-001 | P64 | SP-364 | Polystyrene microparticle, 2.0-2.9 μm SPION |
| P-065-001 | P65 | SP-365 | Silica SPION |
| P-069-001 | P69 | SP-369 | Carboxylated Original coating, 50 nm SPION |
| P-073-001 | P73 | SP-373 | Dextran based coating, 0.13 μm SPION |
| P-074-001 | P74 | SP-374 | Silica Silanol coated with lower acidity SPION |

A particle or surface of the present disclosure may be synthesized, or a particle or surface of the present disclosure may be purchased from a commercial vendor. For example, particles consistent with the present disclosure may be purchased from commercial vendors including Sigma-Aldrich, Life Technologies, Fisher Biosciences, nanoComposix, Nanopartz, Spherotech, and other commercial vendors. In some cases, a particle or surface of the present disclosure may be purchased from a commercial vendor and further modified, coated, or functionalized.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity.

Particles that are consistent with the present disclosure can comprise a wide range of sizes. In some cases, a particle of the present disclosure may be a nanoparticle. In some cases, a nanoparticle of the present disclosure may be from about 10 nm to about 1000 nm in diameter. For example, the nanoparticles disclosed herein can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. In some cases, a nanoparticle may be less than 1000 nm in diameter.

A particle of the present disclosure may be a microparticle. A microparticle may be a particle that is from about 1 μm to about 1000 μm in diameter. For example, the microparticles disclosed here can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. In some cases, a microparticle may be less than 1000 μm in diameter.

The ratio between surface area and mass can be a determinant of a particle's properties in the methods of the instant disclosure. For example, the number and types of biomolecules that a particle adsorbs from a solution may vary with the particle's surface area to mass ratio. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have higher surface area to mass ratios than large particles (e.g., with diameters of 200 nm or more). In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg.

In some cases, a plurality of particles (e.g., of a particle panel) of the compositions and methods described herein may comprise a range of surface area to mass ratios. In some cases, the range of surface area to mass ratios for a plurality of particles is less than 100 cm²/mg, 80 cm²/mg, 60 cm²/mg, 40 cm²/mg, 20 cm²/mg, 10 cm²/mg, 5 cm²/mg, or 2 cm²/mg. In some cases, the surface area to mass ratios for a plurality of particles varies by no more than 40%, 30%, 20%, 10%, 5%, 3%, 2%, or 1% between the particles in the plurality.

In some cases, a plurality of particles (e.g., in a particle panel) may have a wider range of surface area to mass ratios. In some cases, the range of surface area to mass ratios for a plurality of particles is greater than 100 cm²/mg, 150 cm²/mg, 200 cm²/mg, 250 cm²/mg, 300 cm²/mg, 400 cm²/mg, 500 cm²/mg, 800 cm²/mg, 1000 cm²/mg, 1200 cm²/mg, 1500 cm²/mg, 2000 cm²/mg, 3000 cm²/mg, 5000 cm²/mg, 7500 cm²/mg, 10000 cm²/mg, or more. In some cases, the surface area to mass ratios for a plurality of particles (e.g., within a panel) can vary by more than 100%, 200%, 300%, 400%, 500%, 1000%, 10000% or more. In some cases, the plurality of particles with a wide range of surface area to mass ratios comprises at least 2, 3, 4, 5, 6, 7, 8, 9, 10, 12, 15, 20, or more different types of particles.

A particle may comprise a wide array of physical properties. A physical property of a particle may include composition, size, surface charge, hydrophobicity, hydrophilicity, surface functionalization, surface topography, surface curvature, porosity, core material, shell material, shape, and any combination thereof.

A surface functionalization may comprise a polymerizable functional group, a positively or negatively charged functional group, a zwitterionic functional group, an acidic or basic functional group, a polar functional group, or any combination thereof. A surface functionalization may comprise carboxyl groups, hydroxyl groups, thiol groups, cyano groups, cyanate groups, nitro groups, ammonium groups, alkyl groups, imidazolium groups, sulfonium groups, pyridinium groups, pyrrolidinium groups, phosphonium groups, aminopropyl groups, amine groups, boronic acid groups, N-succinimidyl ester groups, PEG groups, streptavidin, methyl ether groups, triethoxylpropylaminosilane groups, PCP groups, citrate groups, lipoic acid groups, BPEI groups, or any combination thereof. A particle from among the plurality of particles may be selected from the group consisting of: micelles, liposomes, iron oxide particles, silver particles, gold particles, palladium particles, quantum dots, platinum particles, titanium particles, silica particles, metal or inorganic oxide particles, synthetic polymer particles, copolymer particles, terpolymer particles, polymeric particles with metal cores, polymeric particles with metal oxide cores, polystyrene sulfonate particles, polyethylene oxide particles, polyoxyethylene glycol particles, polyethylene imine particles, polylactic acid particles, polycaprolactone particles, polyglycolic acid particles, poly(lactide-co-glycolide polymer particles, cellulose ether polymer particles, polyvinylpyrrolidone particles, polyvinyl acetate particles, polyvinylpyrrolidone-vinyl acetate copolymer particles, polyvinyl alcohol particles, acrylate particles, polyacrylic acid particles, crotonic acid copolymer particles, polyethlene phosphonate particles, polyalkylene particles, carboxy vinyl polymer particles, sodium alginate particles, carrageenan particles, xanthan gum particles, gum acacia particles, Arabic gum particles, guar gum particles, pullulan particles, agar particles, chitin particles, chitosan particles, pectin particles, karaya turn particles, locust bean gum particles, maltodextrin particles, amylose particles, corn starch particles, potato starch particles, rice starch particles, tapioca starch particles, pea starch particles, sweet potato starch particles, barley starch particles, wheat starch particles, hydroxypropylated high amylose starch particles, dextrin particles, levan particles, elsinan particles, gluten particles, collagen particles, whey protein isolate particles, casein particles, milk protein particles, soy protein particles, keratin particles, polyethylene particles, polycarbonate particles, polyanhydride particles, polyhydroxyacid particles, polypropylfumerate particles, polycaprolactone particles, polyamine particles, polyacetal particles, polyether particles, polyester particles, poly(orthoester) particles, polycyanoacrylate particles, polyurethane particles, polyphosphazene particles, polyacrylate particles, polymethacrylate particles, polycyanoacrylate particles, polyurea particles, polyamine particles, polystyrene particles, poly(lysine) particles, chitosan particles, dextran particles, poly(acrylamide) particles, derivatized poly(acrylamide) particles, gelatin particles, starch particles, chitosan particles, dextran particles, gelatin particles, starch particles, poly-(3-amino-ester particles, poly(amido amine) particles, poly lactic-co-glycolic acid particles, polyanhydride particles, bioreducible polymer particles, and 2-(3-aminopropylamino)ethanol particles, and any combination thereof.

Particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group.

A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

The present disclosure includes compositions (e.g., particle panels) and methods that comprise two or more particles differing in at least one physicochemical property. A composition or method of the present disclosure may comprise 3 to 6 particles differing in at least one physicochemical property. A composition or method of the present disclosure may comprise 4 to 8 particles differing in at least one physicochemical property. A composition or method of the present disclosure may comprise 4 to 10 particles differing in at least one physicochemical property. A composition or method of the present disclosure may comprise 5 to 12 particles differing in at least one physicochemical property. A composition or method of the present disclosure may comprise 6 to 14 particles differing in at least one physicochemical property. A composition or method of the present disclosure may comprise 8 to 15 particles differing in at least one physicochemical property. A composition or method of the present disclosure may comprise 10 to 20 particles differing in at least one physicochemical property. A composition or method of the present disclosure may comprise at least 2 distinct particle types, at least 3 distinct particle types, at least 4 distinct particle types, at least 5 distinct particle types, at least 6 distinct particle types, at least 7 distinct particle types, at least 8 distinct particle types, at least 9 distinct particle types, at least 10 distinct particle types, at least 11 distinct particle types, at least 12 distinct particle types, at least 13 distinct particle types, at least 14 distinct particle types, at least 15 distinct particle types, at least 20 distinct particle types, at least 25 particle types, or at least 30 distinct particle types.

Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a small molecule functionalization, a macromolecular functionalization, or a combination of two or more such functionalizations. A macromolecular functionalization may comprise a biomacromolecule, such as a protein or a polynucleotide (e.g., a 100-mer DNA molecule). A macromolecular functionalization may be comprise a protein, polynucleotide, or polysaccharide, or may be comparable in size to any of the aforementioned classes of species. For example, a macromolecular functionalization may comprise a volume of at least 6 nm³, at least 8 nm³, at least 12 nm³, at least 15 nm³, at least 20 nm³, at least 30 nm³, at least 50 nm³, at least 80 nm³, at least 120 nm³, at least 180 nm³, at least 300 nm³, at least 500 nm³, at least 800 nm³, at least 1200 nm³, at least 1500 nm³, or at least 2000 nm³. A macromolecular functionalization may comprise a surface area of at least at least 15 nm², at least 20 nm², at least 25 nm², at least 40 nm², at least 80 nm², at least 150 nm², at least 300 nm², at least 500 nm², at least 800 nm², at least 1200 nm², or at least 1500 nm². A macromolecular functionalization may comprise a bait molecule.

A macromolecular functionalization may comprise a specific form of attachment to a particle. A macromolecule may be tethered to a particle via a linker. The linker may hold the macromolecule close to the particle, thereby restricting its motion and reorientation relative to the particle, or may extend the macromolecule away from the particle. The linker may be rigid (e.g., a polyolefin linker) or flexible (e.g., a nucleic acid linker). A linker may be no more than 0.5 nm in length, no more than 1 nm in length, no more than 1.5 nm in length, no more than 2 nm in length, no more than 3 nm in length, no more than 4 nm in length, no more than 5 nm in length, no more than 8 nm in length, or no more than 10 nm in length. A linker may be at least 1 nm in length, at least 2 nm in length, at least 3 nm in length, at least 4 nm in length, at least 5 nm in length, at least 8 nm in length, at least 12 nm in length, at least 15 nm in length, at least 20 nm in length, at least 25 nm in length, or at least 30 nm in length. As such, a surface functionalization on a particle may project beyond a primary corona associated with the particle. A surface functionalization may also be situated beneath or within a biomolecule corona that forms on the particle surface.

A macromolecule may be tethered at a specific location, such as a protein's C-terminus, or may be tethered at a number of possible sites. For example, a peptide may be covalent attached to a particle via any of its surface exposed lysine residues.

A particle may comprise a single surface such as a specific small molecule, or a plurality of surface functionalizations, such as a plurality of different small molecules.

A particle may comprise a high affinity for a particular biomolecule or class of biomolecules. For example, a surface functionalization may comprise a nonpolar moiety (such as an organosilane) that interacts strongly with nonpolar protein functional groups and alpha helices. Analogously, a macromolecular surface functionalization may comprise a peptide (e.g., an antibody) with a high affinity for a specific molecular target.

A particle may comprise a small molecule functionalization. A small molecule functionalization may comprise a mass of fewer than 600 Daltons, fewer than 500 Daltons, fewer than 400 Daltons, fewer than 300 Daltons, fewer than 200 Daltons, or fewer than 100 Daltons. A small molecule functionalization may comprise an ionizable moiety, such as a chemical group with a pKₐ or pK_{b} of less than 6 or 7. A small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

A particle of the present disclosure may be contacted with a biological sample (e.g., a biofluid) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. A single particle type (e.g., a particle of a type listed in TABLE 2) may be contacted to a biological sample. A plurality of particle types (e.g., a plurality of the particle types provided in TABLE 2) may be contacted to a biological sample. The plurality of particle types may be combined and contacted to the biological sample in a single sample volume. The plurality of particle types may be sequentially contacted to a biological sample and separated from the biological sample prior to contacting a subsequent particle type to the biological sample. Protein corona analysis of the biomolecule corona may compress the dynamic range of the analysis compared to a total protein analysis method.

The particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

### Particle Panels

The present disclosure provides compositions and methods of use thereof for assaying a sample for proteins. Compositions described herein include particle panels comprising one or more than one distinct particle types. Particle panels described herein can vary in the number of particle types and the diversity of particle types in a single panel. For example, particles in a panel may vary based on size, polydispersity, shape and morphology, surface charge, surface chemistry and functionalization, and base material. Panels may be incubated with a sample to be analyzed for proteins and protein concentrations. Proteins in the sample adsorb to the surface of the different particle types in the particle panel to form a protein corona. The exact protein and the concentration of protein that adsorbs to a certain particle type in the particle panel may depend on the composition, size, and surface charge of said particle type. Thus, each particle type in a panel may have different protein coronas due to adsorbing a different set of proteins, different concentrations of a particular protein, or a combination thereof. Each particle type in a panel may have mutually exclusive protein coronas or may have overlapping protein coronas. Overlapping protein coronas can overlap in protein identity, in protein concentration, or both.

The present disclosure also provides methods for selecting particle types for inclusion in a panel depending on the sample type. Particle types included in a panel may be a combination of particles that are optimized for removal of highly abundant proteins. Particle types also consistent for inclusion in a panel are those selected for adsorbing particular proteins of interest. The particles can be nanoparticles. The particles can be microparticles. The particles can be a combination of nanoparticles and microparticles.

The particle panels disclosed herein can be used to identify the number of distinct proteins disclosed herein, and/or any of the specific proteins disclosed herein, over a wide dynamic range. For example, the particle panels disclosed herein comprising distinct particle types, can enrich for proteins in a sample, which can be identified using the Proteograph workflow, over the entire dynamic range at which proteins are present in a sample (e.g., a plasma sample). In some cases, a particle panel including any number of distinct particle types disclosed herein, enriches and identifies proteins over a dynamic range of at least 2. In some cases, a particle panel including any number of distinct particle types disclosed herein, enriches and identifies proteins over a dynamic range of at least 3. In some cases, a particle panel including any number of distinct particle types disclosed herein, enriches and identifies proteins over a dynamic range of at least 4. In some cases, a particle panel including any number of distinct particle types disclosed herein, enriches and identifies proteins over a dynamic range of at least 5. In some cases, a particle panel including any number of distinct particle types disclosed herein, enriches and identifies proteins over a dynamic range of at least 6. In some cases, a particle panel including any number of distinct particle types disclosed herein, enriches and identifies proteins over a dynamic range of at least 7. In some cases, a particle panel including any number of distinct particle types disclosed herein, enriches and identifies proteins over a dynamic range of at least 8. In some cases, a particle panel including any number of distinct particle types disclosed herein, enriches and identifies proteins over a dynamic range of at least 9. In some cases, a particle panel including any number of distinct particle types disclosed herein, enriches and identifies proteins over a dynamic range of at least 10. In some cases, a particle panel including any number of distinct particle types disclosed herein, enriches and identifies proteins over a dynamic range of at least 11. In some cases, a particle panel including any number of distinct particle types disclosed herein, enriches and identifies proteins over a dynamic range of at least 12. In some cases, a particle panel including any number of distinct particle types disclosed herein, enriches and identifies proteins over a dynamic range of at least 13. In some cases, a particle panel including any number of distinct particle types disclosed herein, enriches and identifies proteins over a dynamic range of at least 14. In some cases, a particle panel including any number of distinct particle types disclosed herein, enriches and identifies proteins over a dynamic range of at least 15. In some cases, a particle panel including any number of distinct particle types disclosed herein, enriches and identifies proteins over a dynamic range of at least 20. In some cases, a particle panel including any number of distinct particle types disclosed herein, enriches and identifies proteins over a dynamic range of from 2 to 100. In some cases, a particle panel including any number of distinct particle types disclosed herein, enriches and identifies proteins over a dynamic range of from 2 to 20. In some cases, a particle panel including any number of distinct particle types disclosed herein, enriches and identifies proteins over a dynamic range of from 2 to 10. In some cases, a particle panel including any number of distinct particle types disclosed herein, enriches and identifies proteins over a dynamic range of from 2 to 5. In some cases, a particle panel including any number of distinct particle types disclosed herein, enriches and identifies proteins over a dynamic range of from 5 to 10.

A particle panel including any number of distinct particle types disclosed herein, enriches and identifies a single protein or protein group. In some cases, the single protein or protein group may comprise proteins having different post-translational modifications. Introducing a nanoparticle (NP) into a biofluid such as blood plasma may lead to the formation of a selective, specific, and reproducible protein corona at the nano-bio interface driven by the relationship between protein-NP affinity, protein abundance and protein-protein interactions. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the particle panel including any number of distinct particle types disclosed herein, enriches and identifies a single protein or protein group by binding different domains, sequences, or epitopes of the single protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group.

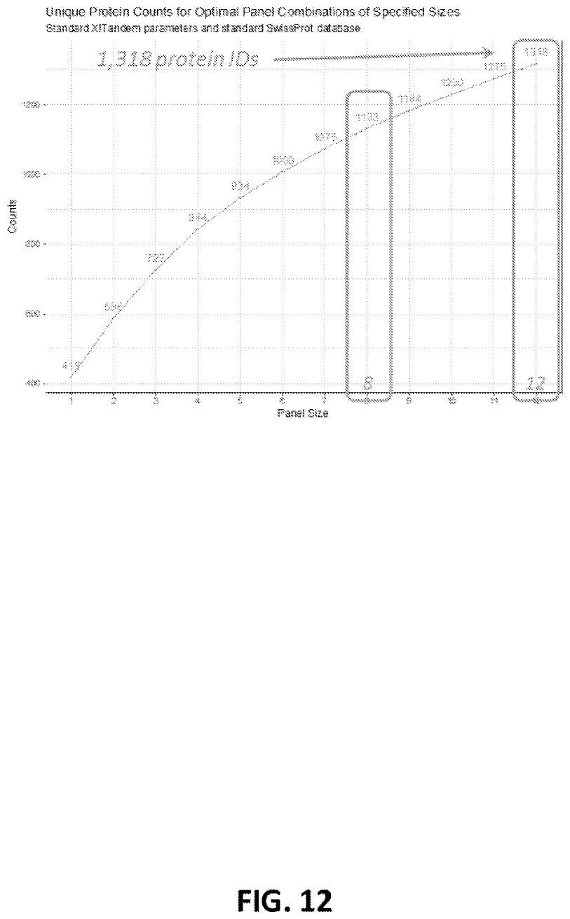

A particle panel can have more than one particle type. Increasing the number of particle types in a panel can be a method for increasing the number of proteins that can be identified in a given sample. An example of how increasing panel size may increase the number of identified proteins is shown in FIG. 12, in which a panel size of one particle type identified 419 different proteins, a panel size of two particle types identified 588 different proteins, a panel size of three particle types identified 727 different proteins, a panel size of four particle types identified 844 proteins, a panel size of five particle types identified 934 different proteins, a panel size of six particle types identified 1008 different proteins, a panel size of seven particle types identified 1075 different proteins, a panel size of eight particle types identified 1133 different proteins, a panel size of nine particle types identified 1184 different proteins, a panel size of 10 particle types identified 1230 different proteins, a panel size of 11 particle types identified 1275 different proteins, and a panel size of 12 particle types identified 1318 different proteins.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA) coated particle. A particle panel consistent with the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

### Protein Analysis Methods

The particles and methods of use thereof disclosed herein can bind a large number of unique biomolecules (e.g., proteins) in a biological sample (e.g., a biofluid). For example, a particle disclosed herein can be incubated with a biological sample to form a protein corona comprising at least 100 unique proteins, at least 120 unique proteins, at least 140 unique proteins, at least 160 unique proteins, at least 180 unique proteins, at least 200 unique proteins, at least 220 unique proteins, at least 240 unique proteins, at least 260 unique proteins, at least 280 unique proteins, at least 300 unique proteins, at least 320 unique proteins, at least 340 unique proteins, at least 360 unique proteins, at least 380 unique proteins, at least 400 unique proteins, at least 420 unique proteins, at least 440 unique proteins, at least 460 unique proteins, at least 480 unique proteins, at least 500 unique proteins, at least 520 unique proteins, at least 540 unique proteins, at least 560 unique proteins, at least 580 unique proteins, at least 600 unique proteins, at least 620 unique proteins, at least 640 unique proteins, at least 660 unique proteins, at least 680 unique proteins, at least 700 unique proteins, at least 720 unique proteins, at least 740 unique proteins, at least 760 unique proteins, at least 780 unique proteins, at least 800 unique proteins, at least 820 unique proteins, at least 840 unique proteins, at least 860 unique proteins, at least 880 unique proteins, at least 900 unique proteins, at least 920 unique proteins, at least 940 unique proteins, at least 960 unique proteins, at least 980 unique proteins, at least 1000 unique proteins, from 100 to 1000 unique proteins, from 150 to 950 unique proteins, from 200 to 900 unique proteins, from 250 to 850 unique proteins, from 300 to 800 unique proteins, from 350 to 750 unique proteins, from 400 to 700 unique proteins, from 450 to 650 unique proteins, from 500 to 600 unique proteins, from 200 to 250 unique proteins, from 250 to 300 unique proteins, from 300 to 350 unique proteins, from 350 to 400 unique proteins, from 400 to 450 unique proteins, from 450 to 500 unique proteins, from 500 to 550 unique proteins, from 550 to 600 unique proteins, from 600 to 650 unique proteins, from 650 to 700 unique proteins, from 700 to 750 unique proteins, from 750 to 800 unique proteins, from 800 to 850 unique proteins, from 850 to 900 unique proteins, from 900 to 950 unique proteins, from 950 to 1000 unique proteins. In some cases, a surface disclosed herein may be incubated with a biological sample to adsorb at least 1000, 2000, 3000, 4000, 5000, 6000, 7000, 8000, 9000, or 10000 unique biomolecules. In some cases, a surface disclosed herein may be incubated with a biological sample to adsorb at most 1000, 2000, 3000, 4000, 5000, 6000, 7000, 8000, 9000, or 10000 unique biomolecules. In some cases, several different types of particles can be used, separately or in combination, to identify large numbers of proteins in a particular biological sample. In other words, particles can be multiplexed in order to bind and identify large numbers of proteins in a biological sample. Protein corona analysis may compress the dynamic range of the analysis compared to a protein analysis of the original sample.

Proteins collected on particles may be subjected to further analysis. A method may comprise collecting a biomolecule corona or a subset of biomolecules from a biomolecule corona. The collected biomolecule corona or the collected subset of biomolecules from the biomolecule corona may be subjected to further particle-based analysis (e.g., particle adsorption). The collected biomolecule corona or the collected subset of biomolecules from the biomolecule corona may be purified or fractionated (e.g., by a chromatographic method). The collected biomolecule corona or the collected subset of biomolecules from the biomolecule corona may be analyzed (e.g., by mass spectrometry).

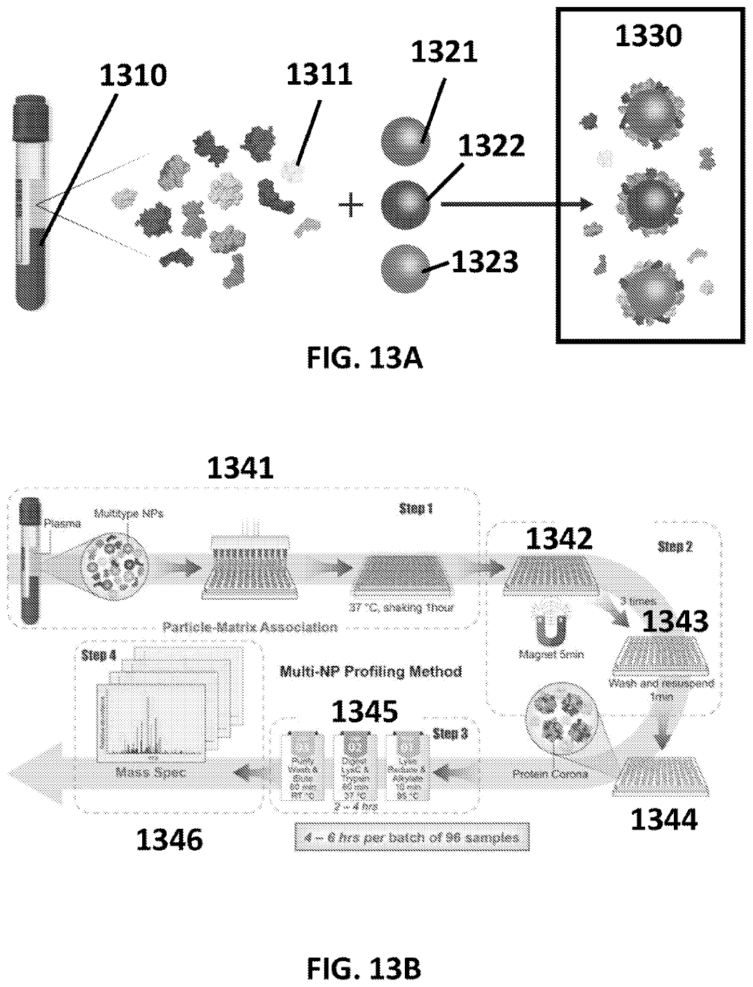

FIGS. 13A-B provide an example of a method consistent with the present disclosure. FIG. 13A illustrates a schematic of the formation of protein corona, wherein a plurality of particles 1321, 1322, & 1323 particles are contacted with a biological sample 1310 comprising biomolecules molecules 1311, and wherein each particle adsorbs a plurality of biomolecules from the biological sample to its surface 1330. The different particles may be distinct particle types (depicted in the center of the figure, with the top, middle, and bottom spheres representing the three distinct particle types), such that each particle differs from the other particles by at least one physicochemical property. This difference in physicochemical properties can lead to the formation of different protein corona compositions on the particle surfaces FIG. 13B shows a biomolecule corona (e.g., protein corona) analysis workflow which includes: (1341) particle-plasma incubation and protein corona formation; (1342) particle collection (e.g., with a magnet); (1343) washing away solution and analytes not adsorbed to the particles; (1434) resuspension of the particles; (1435) digestion of corona proteins; and (1346) liquid chromatography-mass spectrometry analysis (LC-MS). In this example, each plasma-NP well is a sample for a total of 96 samples per plate.

Protein corona analysis may comprise an automated component. For example, an automated instrument may contact a sample with a particle or particle panel, identify proteins on the particle or particle panel (e.g., digest the proteins on the particle or particle panel and perform mass spectrometric analysis), and generate data for identifying a specific biomolecule or a biological state of a sample. The automated instrument may divide a sample into a plurality of volumes, and perform analysis on each volume. The automated instrument may analyze multiple separate samples, for example by disposing multiple samples within multiple wells in a well plate, and performing parallel analysis on each sample.

The particle panels disclosed herein can be used to identifying a number of proteins, peptides, protein groups, or protein classes using a protein analysis workflow described herein (e.g., a protein corona analysis workflow). Protein corona analysis may comprise contacting a sample to distinct particle types (e.g., a particle panel), forming biomolecule corona on the distinct particle types, and identifying the biomolecules in the biomolecule corona (e.g., by mass spectrometry). Feature intensities, as disclosed herein, refers to the intensity of a discrete spike (“feature”) seen on a plot of mass to charge ratio versus intensity from a mass spectrometry run of a sample. These features can correspond to variably ionized fragments of peptides and/or proteins. Using the data analysis methods described herein, feature intensities can be sorted into protein groups. Protein groups refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). Alternatively, if the peptide sequence is unique to a single protein (Protein 1), a protein group could be the “ZZX” protein group having one member (Protein 1). Each protein group can be supported by more than one peptide sequence. Protein detected or identified according to the instant disclosure can refer to a distinct protein detected in the sample (e.g., distinct relative other proteins detected using mass spectrometry). Thus, analysis of proteins present in distinct coronas corresponding to the distinct particle types in a particle panel yields a high number of feature intensities. This number decreases as feature intensities are processed into distinct peptides, further decreases as distinct peptides are processed into distinct proteins, and further decreases as peptides are grouped into protein groups (two or more proteins that share a distinct peptide sequence).

The methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. Thus, the methods disclosed herein provide for isolating or separating a particle type from unbound protein in a sample. A particle type may be separated by a variety of means, including but not limited to magnetic separation, centrifugation, filtration, or gravitational separation. Particle panels may be incubated with a plurality of spatially isolated samples, wherein each spatially isolated sample is in a well in a well plate (e.g., a 96-well plate). After incubation, the particle types in each of the wells of the well plate can be separated from unbound protein present in the spatially isolated samples by placing the entire plate on a magnet. This simultaneously pulls down the superparamagnetic particles in the particle panel. The supernatant in each sample can be removed to remove the unbound protein. These steps (incubate, pull down) can be repeated to effectively wash the particles, thus removing residual background unbound protein that may be present in a sample. This is one example, but one of skill in the art could envision numerous other scenarios in which superparamagnetic particles are rapidly isolated from one or more than one spatially isolated samples at the same time.

The methods and compositions of the present disclosure provide identification and measurement of particular proteins in the biological samples by processing of the proteomic data via digestion of coronas formed on the surface of particles. Examples of proteins that can be identified and measured include highly abundant proteins, proteins of medium abundance, and low-abundance proteins. A low abundance protein may be present in a sample at concentrations at or below about 10 ng/mL. A high abundance protein may be present in a sample at concentrations at or above about 10 μg/mL. A protein of moderate abundance may be present in a sample at concentrations between about 10 ng/mL and about 10 μg/mL. Examples of proteins that are highly abundant proteins include albumin, IgG, and the top 14 proteins in abundance that contribute 95% of the analyte mass in plasma. Additionally, any proteins that may be purified using a conventional depletion column may be directly detected in a sample using the particle panels disclosed herein. Examples of proteins may be any protein listed in published databases such as Keshishian et al. (Mol Cell Proteomics. 2015 Sep. 14(9):2375-93. doi: 10.1074/mcp.M114.046813. Epub 2015 Feb. 27.), Farr et al. (J Proteome Res. 2014 Jan. 3; 13(1):60-75. doi: 10.1021/pr4010037. Epub 2013 Dec. 6.), or Pernemalm et al. (Expert Rev Proteomics. 2014 August; 11(4):431-48. doi: 10.1586/14789450.2014.901157. Epub 2014 Mar. 24.).

Examples of proteins that can be measured and identified using the methods and compositions disclosed herein include albumin, IgG, lysozyme, CEA, HER-2/neu, bladder tumor antigen, thyroglobulin, alpha-fetoprotein, PSA, CA125, CA19.9, CA 15.3, leptin, prolactin, osteopontin, IGF-II, CD98, fascin, sPigR, 14-3-3 eta, troponin I, B-type natriuretic peptide, BRCA1, c-Myc, IL-6, fibrinogen. EGFR, gastrin, PH, G-CSF, desmin. NSE, FSH, VEGF, P21, PCNA, calcitonin, PR, CA125, LH, somatostatin. S100, insulin. alpha-prolactin, ACTH, Bc1-2, ER alpha, Ki-67, p53, cathepsin D, beta catenin. VWF, CD15, k-ras, caspase 3, EPN, CD10, FAS, BRCA2. CD30L, CD30, CGA, CRP, prothrombin, CD44, APEX, transferrin, GM-CSF, E-cadherin, IL-2, Bax, IFN-gamma, beta-2-MG, TNF alpha, c-erbB-2, trypsin, cyclin D1, MG B, HG-1, YKL-40, S-gamma, NESP-55, netrin-1, geminin, GADD45A, CDK-6, CCL21, BrMS1, 17betaHDI, PDGFRA, Pcaf, CCL5, MMP3, claudin-4, and claudin-3. In some cases, other examples of proteins that can be measured and identified using the particle panels disclosed herein are any proteins or protein groups listed in the open targets database for a particular disease indication of interest (e.g., prostate cancer, lung cancer, or Alzheimer's disease).

The methods and compositions disclosed herein may also elucidate protein classes or interactions of the protein classes. A protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); or proteins bearing a post-translational modification (e.g., ubiquitinated or citrullinated proteins). A protein class may contain at least 2 proteins, 5 proteins, 10 proteins, 20 proteins, 40 proteins, 60 proteins, 80 proteins, 100 proteins, 150 proteins, 200 proteins, or more.

The proteomic data of the biological sample can be identified, measured, and quantified using a number of different analytical techniques. For example, proteomic data can be generated using SDS-PAGE or any gel-based separation technique. Peptides and proteins can also be identified, measured, and quantified using an immunoassay, such as ELISA. Alternatively, proteomic data can be identified, measured, and quantified using mass spectrometry, high performance liquid chromatography, LC-MS/MS, Edman Degradation, immunoaffinity techniques, methods disclosed in EP3548652, WO2019083856, WO2019133892, each of which is incorporated herein by reference in its entirety, and other protein separation techniques.

An assay may comprise protein collection of particles, protein digestion, and mass spectrometric analysis (e.g., MS, LC-MS, LC-MS/MS). The digestion may comprise chemical digestion, such as by cyanogen bromide or 2-Nitro-5-thiocyanatobenzoic acid (NTCB). The digestion may comprise enzymatic digestion, such as by trypsin or pepsin. The digestion may comprise enzymatic digestion by a plurality of proteases. The digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. The digestion may cleave peptides at random positions. The digestion may cleave peptides at a specific position (e.g., at methionines) or sequence (e.g., glutamate-histidine-glutamate). The digestion may enable similar proteins to be distinguished. For example, an assay may resolve 8 distinct proteins as a single protein group with a first digestion method, and as 8 separate proteins with distinct signals with a second digestion method. The digestion may generate an average peptide fragment length of 8 to 15 amino acids. The digestion may generate an average peptide fragment length of 12 to 18 amino acids. The digestion may generate an average peptide fragment length of 15 to 25 amino acids. The digestion may generate an average peptide fragment length of 20 to 30 amino acids. The digestion may generate an average peptide fragment length of 30 to 50 amino acids.

An assay may rapidly generate and analyze proteomic data. Beginning with an input biological sample (e.g., a buccal or nasal smear, plasma, or tissue), an assay of the present disclosure may generate and analyze proteomic data in less than 7 hours. Beginning with an input biological sample, an assay of the present disclosure may generate and analyze proteomic data in 5-7 hours. Beginning with an input biological sample, an assay of the present disclosure may generate and analyze proteomic data in less than 5 hours. Beginning with an input biological sample, an assay of the present disclosure may generate and analyze proteomic data in 3-5 hours. Beginning with an input biological sample, an assay of the present disclosure may generate and analyze proteomic data in 2-4 hours. Beginning with an input biological sample, an assay of the present disclosure may generate and analyze proteomic data in 2-3 hours. Beginning with an input biological sample, an assay of the present disclosure may generate and analyze proteomic data in less than 3 hours. Beginning with an input biological sample, an assay of the present disclosure may generate and analyze proteomic data in less than 2 hours. The analyzing may comprise identifying a protein group. The analyzing may comprise identifying a protein class. The analyzing may comprise quantifying an abundance of a biomolecule, a peptide, a protein, protein group, or a protein class. The analyzing may comprise identifying a ratio of abundances of two biomolecules, peptides, proteins, protein groups, or protein classes. The analyzing may comprise identifying a biological state.

### Kits

Provided herein are kits comprising compositions of the present disclosure that may be used to perform the methods of the present disclosure. A kit may comprise one or more particle types to interrogate a sample to identify a biological state of a sample. In some cases, a kit may comprise a particle type provided in TABLE 2. A kit may comprise a reagent for functionalizing a particle (e.g., a reagent for tethering a small molecule functionalization to a particle surface). The kit may be pre-packaged in discrete aliquots. In some cases, the kit can comprise a plurality of different particle types that can be used to interrogate a sample. The plurality of particle types can be pre-packaged where each particle type of the plurality is packaged separately. Alternately, the plurality of particle types can be packaged together to contain combination of particle types in a single package. A particle may be provided in dried (e.g., lyophilized) form, or may be provided in a suspension or solution. The particles may be provided in a well plate. For example, a kit may contain a 24-384 well plate with the particles sealed within the wells. Two wells in such a well plate may contain different particles or concentrations of particles. Two wells may comprise different buffers or chemical conditions. For example, a well plate may be provided with different particles in each row of wells and different buffers in each column of rows. A well may be sealed by a removable covering. For example, a kit may comprise a well plate comprising a plastic slip covering a plurality of wells. A well may be sealed by a pierceable covering. For example, a well may be covered by a septum that a needle can pierce to facilitate sample movement into and out of the well.

In some aspects, the present disclosure describes a kit for enriching a biological sample. In some cases, the kit comprises a first substance configured to specifically bind to a first set of biomolecule targets. In some cases, the kit comprises a second substance configured to adsorb a second set of biomolecule targets. In some cases, the kit comprises a third substance configured to adsorb a third set of biomolecule targets.

In some cases, the first substance is a resin or a particle. In some cases, the first substance comprises a specific binding moiety configured to bind to the first set of biomolecule targets. In some cases, the first substance is configured to specifically bind to at least one of: albumin, IgG, IgA, IgM, IgD, IgE, IgG (light chains), alpha-1-acid glycoprotein, fibrinogen, haptoglobin, alpha-1-antitrypsin, alpha-2-macroglobulin, transferrin, and apolipoprotein A-1.

In some cases, the kit may further comprise a fourth substance configured to non-specifically bind to a fourth set of biomolecule targets. In some cases, the kit may further comprise a fifth substance configured to non-specifically bind to a fifth set of biomolecule targets. The kit may comprise various number of substances configured to non-specifically bind to sets of biomolecule targets.

In some cases, the kit may further comprise a fifth substance configured to specifically bind to a fifth set of biomolecule targets. In some cases, the kit may further comprise a sixth substance configured to specifically bind to a sixth set of biomolecule targets. The kit may comprise various number of substances configured to specifically bind to sets of biomolecule targets.

In some cases, the second substance comprises a plurality of domains. In some cases, each domain in the plurality of domains is configured to non-specifically bind to a distinct subset in the second set of biomolecule targets. In some cases, the second substance comprises a particle surface. In some cases, the plurality of domains comprises a plurality of surface regions on the particle surface. For example, the second substance may comprise a particle comprising two or more distinct regions having distinct physicochemical properties. In some cases, the second substance comprises a plurality of particle surfaces, and the plurality of particle surfaces are disposed on a plurality of particles. For example, the second substance may be a mixture of two or more particles disclosed herein.

In some cases, the kit comprises a chamber or a well having the first substance, the second substance, and the third substance disposed therein. The kit may comprise a chamber or a well of various shapes and forms that are configured to receive a biological sample. For example, in some cases, the chamber comprises a column. In some cases, the chamber comprises a microfluidic channel. In some cases, a surface region of the well comprises the first substance.

### Samples

The present disclosure provides a range of samples that can be assayed using the particles and the methods provided herein. A sample may be a biological sample (e.g., a sample derived from a living organism). A sample may comprise a cell or be cell-free. A sample may comprise a biofluid, such as blood, serum, plasma, urine, or cerebrospinal fluid (CSF). Samples consistent with the present disclosure include biological samples from a subject. The subject may be a human or a non-human animal. Said biological samples can contain a plurality of proteins or proteomic data, which may be analyzed after adsorption of proteins to the surface of the various sensor element (e.g., particle) types in a panel and subsequent digestion of protein coronas. Proteomic data can comprise nucleic acids, peptides, or proteins. A biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract.

A wide range of samples are compatible for use within the methods and compositions of the present disclosure. The biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. The biological sample may comprise multiple biological samples (e.g., pooled plasma from multiple subjects, or multiple tissue samples from a single subject). The biological sample may comprise a single type of biofluid or biomaterial from a single source.

The biological sample may be diluted or pre-treated. The biological sample may undergo depletion (e.g., the biological sample comprises serum) prior to or following contact with a particle or plurality of particles. The biological sample may also undergo physical (e.g., homogenization or sonication) or chemical treatment prior to or following contact with a particle or plurality of particles. The biological sample may be diluted prior to or following contact with a particle or plurality of particles. The dilution medium may comprise buffer or salts, or be purified water (e.g., distilled water). Different partitions of a biological sample may undergo different degrees of dilution. A biological sample or a portion thereof may undergo a 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution.

In some cases, the biological sample may comprise a plurality of biomolecules. In some cases, a plurality of biomolecules may comprise polyamino acids. In some cases, the polyamino acids comprise peptides, proteins, or a combination thereof. In some cases, the plurality of biomolecules may comprise nucleic acids, carbohydrates, polyamino acids, or any combination thereof.

### Biological States

The compositions and methods disclosed herein can be used to identify various biological states in a particular biological sample. For example, a biological state can refer to an elevated or low level of a particular protein or a set of proteins. In other examples, a biological state can refer to identification of a disease, such as cancer. The particles and methods of us thereof can be used to distinguish between two biological states. The two biological states may be related diseases states (e.g., two HRAS mutant colon cancers or different stages of a type of a cancer). The two biological states may be different phases of a disease, such as pre-Alzheimer's and mild Alzheimer's. The two biological states may be distinguished with a high degree of accuracy (e.g., the percentage of accurately identified biological states among a population of samples). For example, the compositions and methods of the present disclosure may distinguish two biological states with at least 60% accuracy, at least 70% accuracy, at least 75% accuracy at least 80% accuracy, at least 85% accuracy, at least 90% accuracy, at least 95% accuracy, at least 98% accuracy, or at least 99% accuracy. The two biological states may be distinguished with a high degree of specificity (e.g., the rate at which negative results are correctly identified among a population of samples). For example, the compositions and methods of the present disclosure may distinguish two biological states with at least 60% specificity, at least 70% specificity, at least 75% specificity at least 80% specificity, at least 85% specificity, at least 90% specificity, at least 95% specificity, at least 98% specificity, or at least 99% specificity.

The methods, compositions, and systems described herein can be used to determine a disease state, and/or prognose or diagnose a disease or disorder. The diseases or disorders contemplated include, but are not limited to, for example, cancer, cardiovascular disease, endocrine disease, inflammatory disease, a neurological disease and the like.

The methods, compositions, and systems described herein can be used to determine, prognose, and/or diagnose a cancer disease state. The term “cancer” is meant to encompass any cancer, neoplastic and preneoplastic disease that is characterized by abnormal growth of cells, including tumors and benign growths. Cancer may, for example, be lung cancer, pancreatic cancer, or skin cancer. In many cases, the methods, compositions and systems described herein are not only able to diagnose cancer (e.g. determine if a subject (a) does not have cancer, (b) is in a pre-cancer development stage, (c) is in early stage of cancer, (d) is in a late stage of cancer) but are able to determine the type of cancer.

The methods, compositions, and systems of the present disclosure can additionally be used to detect other cancers, such as acute lymphoblastic leukemia (ALL); acute myeloid leukemia (AML); cancer in adolescents; adrenocortical carcinoma; childhood adrenocortical carcinoma; unusual cancers of childhood; AIDS-related cancers; kaposi sarcoma (soft tissue sarcoma); AIDS-related lymphoma (lymphoma); primary cns lymphoma (lymphoma); anal cancer; appendix cancer—see gastrointestinal carcinoid tumors; astrocytomas, childhood (brain cancer); atypical teratoid/rhabdoid tumor, childhood, central nervous system (brain cancer); basal cell carcinoma of the skin—see skin cancer; bile duct cancer; bladder cancer; childhood bladder cancer; bone cancer (includes ewing sarcoma and osteosarcoma and malignant fibrous histiocytoma); brain tumors; breast cancer; childhood breast cancer; bronchial tumors, childhood; burkitt lymphoma—see non-hodgkin lymphoma; carcinoid tumor (gastrointestinal); childhood carcinoid tumors; carcinoma of unknown primary; childhood carcinoma of unknown primary; cardiac (heart) tumors, childhood; central nervous system; atypical teratoid/rhabdoid tumor, childhood (brain cancer); embryonal tumors, childhood (brain cancer); germ cell tumor, childhood (brain cancer); primary cns lymphoma; cervical cancer; childhood cervical cancer; childhood cancers; cancers of childhood, unusual; cholangiocarcinoma—see bile duct cancer; chordoma, childhood; chronic lymphocytic leukemia (CLL); chronic myelogenous leukemia (CIVIL); chronic myeloproliferative neoplasms; colorectal cancer; childhood colorectal cancer; craniopharyngioma, childhood (brain cancer); cutaneous t-cell lymphoma—see lymphoma (mycosis fungoides and sézary syndrome); ductal carcinoma in situ (DCIS)—see breast cancer; embryonal tumors, central nervous system, childhood (brain cancer); endometrial cancer (uterine cancer); ependymoma, childhood (brain cancer); esophageal cancer; childhood esophageal cancer; esthesioneuroblastoma (head and neck cancer); ewing sarcoma (bone cancer); extracranial germ cell tumor, childhood; extragonadal germ cell tumor; eye cancer; childhood intraocular melanoma; intraocular melanoma; retinoblastoma; fallopian tube cancer; fibrous histiocytoma of bone, malignant, and osteosarcoma; gallbladder cancer; gastric (stomach) cancer; childhood gastric (stomach) cancer; gastrointestinal carcinoid tumor; gastrointestinal stromal tumors (GIST) (soft tissue sarcoma); childhood gastrointestinal stromal tumors; germ cell tumors; childhood central nervous system germ cell tumors (brain cancer); childhood extracranial germ cell tumors; extragonadal germ cell tumors; ovarian germ cell tumors; testicular cancer; gestational trophoblastic disease; hairy cell leukemia; head and neck cancer; heart tumors, childhood; hepatocellular (liver) cancer; histiocytosis, langerhans cell; hodgkin lymphoma; hypopharyngeal cancer (head and neck cancer); intraocular melanoma; childhood intraocular melanoma; islet cell tumors, pancreatic neuroendocrine tumors; kaposi sarcoma (soft tissue sarcoma); kidney (renal cell) cancer; langerhans cell histiocytosis; laryngeal cancer (head and neck cancer); leukemia; lip and oral cavity cancer (head and neck cancer); liver cancer; lung cancer (non-small cell and small cell); childhood lung cancer; lymphoma; male breast cancer; malignant fibrous histiocytoma of bone and osteosarcoma; melanoma; childhood melanoma; melanoma, intraocular (eye); childhood intraocular melanoma; merkel cell carcinoma (skin cancer); mesothelioma, malignant; childhood mesothelioma; metastatic cancer; metastatic squamous neck cancer with occult primary (head and neck cancer); midline tract carcinoma with nut gene changes; mouth cancer (head and neck cancer); multiple endocrine neoplasia syndromes; multiple myeloma/plasma cell neoplasms; mycosis fungoides (lymphoma); myelodysplastic syndromes, myelodysplastic/myeloproliferative neoplasms; myelogenous leukemia, chronic (cml); myeloid leukemia, acute (aml); myeloproliferative neoplasms, chronic; nasal cavity and paranasal sinus cancer (head and neck cancer); nasopharyngeal cancer (head and neck cancer); neuroblastoma; non-hodgkin lymphoma; non-small cell lung cancer; oral cancer, lip and oral cavity cancer and oropharyngeal cancer (head and neck cancer); osteosarcoma and malignant fibrous histiocytoma of bone; ovarian cancer; childhood ovarian cancer; pancreatic cancer; childhood pancreatic cancer; pancreatic neuroendocrine tumors (islet cell tumors); papillomatosis (childhood laryngeal); paraganglioma; childhood paraganglioma; paranasal sinus and nasal cavity cancer (head and neck cancer); parathyroid cancer; penile cancer; pharyngeal cancer (head and neck cancer); pheochromocytoma; childhood pheochromocytoma; pituitary tumor; plasma cell neoplasm/multiple myeloma; pleuropulmonary blastoma; pregnancy and breast cancer; primary central nervous system (CNS) lymphoma; primary peritoneal cancer; prostate cancer; rectal cancer; recurrent cancer; renal cell (kidney) cancer; retinoblastoma; rhabdomyosarcoma, childhood (soft tissue sarcoma); salivary gland cancer (head and neck cancer); sarcoma; childhood rhabdomyosarcoma (soft tissue sarcoma); childhood vascular tumors (soft tissue sarcoma); ewing sarcoma (bone cancer); kaposi sarcoma (soft tissue sarcoma); osteosarcoma (bone cancer); soft tissue sarcoma; uterine sarcoma; sézary syndrome (lymphoma); skin cancer; childhood skin cancer; small cell lung cancer; small intestine cancer; soft tissue sarcoma; squamous cell carcinoma of the skin—see skin cancer; squamous neck cancer with occult primary, metastatic (head and neck cancer); stomach (gastric) cancer; childhood stomach (gastric) cancer; t-cell lymphoma, cutaneous—see lymphoma (mycosis fungoides and sézary syndrome); testicular cancer; childhood testicular cancer; throat cancer (head and neck cancer); nasopharyngeal cancer; oropharyngeal cancer; hypopharyngeal cancer; thymoma and thymic carcinoma; thyroid cancer; transitional cell cancer of the renal pelvis and ureter (kidney (renal cell) cancer); carcinoma of unknown primary; childhood cancer of unknown primary; unusual cancers of childhood; ureter and renal pelvis, transitional cell cancer (kidney (renal cell) cancer; urethral cancer; uterine cancer, endometrial; uterine sarcoma; vaginal cancer; childhood vaginal cancer; vascular tumors (soft tissue sarcoma); vulvar cancer; wilms tumor and other childhood kidney tumors; or cancer in young adults.

The methods, compositions, and systems of the present disclosure may be used to detect a cardiovascular disease state. As used herein, the terms “cardiovascular disease” (CVD) or “cardiovascular disorder” are used to classify numerous conditions affecting the heart, heart valves, and vasculature (e.g., veins and arteries) of the body and encompasses diseases and conditions including, but not limited to atherosclerosis, myocardial infarction, acute coronary syndrome, angina, congestive heart failure, aortic aneurysm, aortic dissection, iliac or femoral aneurysm, pulmonary embolism, atrial fibrillation, stroke, transient ischemic attack, systolic dysfunction, diastolic dysfunction, myocarditis, atrial tachycardia, ventricular fibrillation, endocarditis, peripheral vascular disease, and coronary artery disease (CAD). Further, the term cardiovascular disease refers to conditions in subjects that ultimately have a cardiovascular event or cardiovascular complication, referring to the manifestation of an adverse condition in a subject brought on by cardiovascular disease, such as sudden cardiac death or acute coronary syndrome, including, but not limited to, myocardial infarction, unstable angina, aneurysm, stroke, heart failure, non-fatal myocardial infarction, stroke, angina pectoris, transient ischemic attacks, aortic aneurysm, aortic dissection, cardiomyopathy, abnormal cardiac catheterization, abnormal cardiac imaging, stent or graft revascularization, risk of experiencing an abnormal stress test, risk of experiencing abnormal myocardial perfusion, and death.

As used herein, the ability to detect, diagnose or prognose cardiovascular disease, for example, atherosclerosis, can include determining if the patient is in a pre-stage of cardiovascular disease, has developed early, moderate or severe forms of cardiovascular disease, or has suffered one or more cardiovascular event or complication associated with cardiovascular disease.

Atherosclerosis (also known as arteriosclerotic vascular disease or ASVD) is a cardiovascular disease in which an artery-wall thickens as a result of invasion and accumulation and deposition of arterial plaques containing white blood cells on the innermost layer of the walls of arteries resulting in the narrowing and hardening of the arteries. The arterial plaque is an accumulation of macrophage cells or debris, and contains lipids (cholesterol and fatty acids), calcium and a variable amount of fibrous connective tissue. Diseases associated with atherosclerosis include, but are not limited to, atherothrombosis, coronary heart disease, deep venous thrombosis, carotid artery disease, angina pectoris, peripheral arterial disease, chronic kidney disease, acute coronary syndrome, vascular stenosis, myocardial infarction, aneurysm or stroke. In one embodiment the automated apparatuses, compositions, and methods of the present disclosure may distinguish the different stages of atherosclerosis, including, but not limited to, the different degrees of stenosis in a subject.

In some cases, the disease or disorder detected by the methods, compositions, or systems of the present disclosure is an endocrine disease. The term “endocrine disease” is used to refer to a disorder associated with dysregulation of endocrine system of a subject. Endocrine diseases may result from a gland producing too much or too little of an endocrine hormone causing a hormonal imbalance, or due to the development of lesions (such as nodules or tumors) in the endocrine system, which may or may not affect hormone levels. Suitable endocrine diseases able to be treated include, but are not limited to, e.g., Acromegaly, Addison's Disease, Adrenal Cancer, Adrenal Disorders, Anaplastic Thyroid Cancer, Cushing's Syndrome, De Quervain's Thyroiditis, Diabetes, Follicular Thyroid Cancer, Gestational Diabetes, Goiters, Graves' Disease, Growth Disorders, Growth Hormone Deficiency, Hashimoto's Thyroiditis, Hurthle Cell Thyroid Cancer, Hyperglycemia, Hyperparathyroidism, Hyperthyroidism, Hypoglycemia, Hypoparathyroidism, Hypothyroidism, Low Testosterone, Medullary Thyroid Cancer, MEN 1, MEN 2A, MEN 2B, Menopause, Metabolic Syndrome, Obesity, Osteoporosis, Papillary Thyroid Cancer, Parathyroid Diseases, Pheochromocytoma, Pituitary Disorders, Pituitary Tumors, Polycystic Ovary Syndrome, Prediabetes, Silent, Thyroiditis, Thyroid Cancer, Thyroid Diseases, Thyroid Nodules, Thyroiditis, Turner Syndrome, Type 1 Diabetes, Type 2 Diabetes, and the like.

In some cases, the disease or disorder detected by methods, compositions, or systems of the present disclosure is an inflammatory disease. As referred to herein, inflammatory disease refers to a disease caused by uncontrolled inflammation in the body of a subject. Inflammation is a biological response of the subject to a harmful stimulus which may be external or internal such as pathogens, necrosed cells and tissues, irritants etc. However, when the inflammatory response becomes abnormal, it results in self-tissue injury and may lead to various diseases and disorders. Inflammatory diseases can include, but are not limited to, asthma, glomerulonephritis, inflammatory bowel disease, rheumatoid arthritis, hypersensitivities, pelvic inflammatory disease, autoimmune diseases, arthritis; necrotizing enterocolitis (NEC), gastroenteritis, pelvic inflammatory disease (PID), emphysema, pleurisy, pyelitis, pharyngitis, angina, acne vulgaris, urinary tract infection, appendicitis, bursitis, colitis, cystitis, dermatitis, phlebitis, rhinitis, tendonitis, tonsillitis, vasculitis, autoimmune diseases; celiac disease; chronic prostatitis, hypersensitivities, reperfusion injury; sarcoidosis, transplant rejection, vasculitis, interstitial cystitis, hay fever, periodontitis, atherosclerosis, psoriasis, ankylosing spondylitis, juvenile idiopathic arthritis, Behcet's disease, spondyloarthritis, uveitis, systemic lupus erythematosus, and cancer. For example, the arthritis includes rheumatoid arthritis, psoriatic arthritis, osteoarthritis or juvenile idiopathic arthritis, and the like.

The methods, compositions, and systems of the present disclosure may detect a neurological disease state. Neurological disorders or neurological diseases are used interchangeably and refer to diseases of the brain, spine and the nerves that connect them. Neurological diseases include, but are not limited to, brain tumors, epilepsy, Parkinson's disease, Alzheimer's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease (including but are not limited to, multiple sclerosis, Devic's disease (i.e. neuromyelitis optica), central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, and anti-MAG peripheral neuropathy) and the like. Neurological disorders also include immune-mediated neurological disorders (IMNDs), which include diseases with at least one component of the immune system reacts against host proteins present in the central or peripheral nervous system and contributes to disease pathology. IMNDs may include, but are not limited to, demyelinating disease, paraneoplastic neurological syndromes, immune-mediated encephalomyelitis, immune-mediated autonomic neuropathy, myasthenia gravis, autoantibody-associated encephalopathy, and acute disseminated encephalomyelitis.

Methods, systems, and/or apparatuses of the present disclosure may be able to accurately distinguish between patients with or without Alzheimer's disease. These may also be able to detect patients who are pre-symptomatic and may develop Alzheimer's disease several years after the screening. This provides advantages of being able to treat a disease at a very early stage, even before development of the disease.

The methods, compositions, and systems of the present disclosure can detect a pre-disease stage of a disease or disorder. A pre-disease stage is a stage at which the patient has not developed any signs or symptoms of the disease. A pre-cancerous stage would be a stage in which cancer or tumor or cancerous cells have not be identified within the subject. A pre-neurological disease stage would be a stage in which a person has not developed one or more symptom of the neurological disease. The ability to diagnose a disease before one or more sign or symptom of the disease is present allows for close monitoring of the subject and the ability to treat the disease at a very early stage, increasing the prospect of being able to halt progression or reduce the severity of the disease.

The methods, compositions, and systems of the present disclosure may detect the early stages of a disease or disorder. Early stages of the disease can refer to when the first signs or symptoms of a disease may manifest within a subject. The early stage of a disease may be a stage at which there are no outward signs or symptoms. For example, in Alzheimer's disease an early stage may be a pre-Alzheimer's stage in which no symptoms are detected yet the patient will develop Alzheimer's months or years later.

Identifying a disease in either pre-disease development or in the early states may often lead to a higher likelihood for a positive outcome for the patient. For example, diagnosing cancer at an early stage (stage 0 or stage 1) can increase the likelihood of survival by over 80%. Stage 0 cancer can describe a cancer before it has begun to spread to nearby tissues. This stage of cancer is often highly curable, usually by removing the entire tumor with surgery. Stage 1 cancer may usually be a small cancer or tumor that has not grown deeply into nearby tissue and has not spread to lymph nodes or other parts of the body.

In some cases, the methods, compositions, and systems of the present disclosure are able to detect intermediate stages of the disease. Intermediate states of the disease describe stages of the disease that have passed the first signs and symptoms and the patient is experiencing one or more symptom of the disease. For example, for cancer, stage II or III cancers are considered intermediate stages, indicating larger cancers or tumors that have grown more deeply into nearby tissue. In some instances, stage II or III cancers may have also spread to lymph nodes but not to other parts of the body.

Further, the methods, compositions, and systems of the present disclosure may be able to detect late or advanced stages of the disease. Late or advanced stages of the disease may also be called “severe” or “advanced” and usually indicates that the subject is suffering from multiple symptoms and effects of the disease. For example, severe stage cancer includes stage IV, where the cancer has spread to other organs or parts of the body and is sometimes referred to as advanced or metastatic cancer.

The methods of the present disclosure can include processing the biomolecule corona data of a sample against a collection of biomolecule corona datasets representative of a plurality of diseases and/or a plurality of disease states to determine if the sample indicates a disease and/or disease state. For example, samples can be collected from a population of subjects over time. Once the subjects develop a disease or disorder, the present disclosure allows for the ability to characterize and detect the changes in biomolecule fingerprints over time in the subject by computationally analyzing the biomolecule fingerprint of the sample from the same subject before they have developed a disease to the biomolecule fingerprint of the subject after they have developed the disease. Samples can also be taken from cohorts of patients who all develop the same disease, allowing for analysis and characterization of the biomolecule fingerprints that are associated with the different stages of the disease for these patients (e.g. from pre-disease to disease states).

In some cases, the methods, compositions, and systems of the present disclosure are able to distinguish not only between different types of diseases, but also between the different stages of the disease (e.g. early stages of cancer). This can comprise distinguishing healthy subjects from pre-disease state subjects. The pre-disease state may be stage 0 or stage 1 cancer, a neurodegenerative disease, dementia, a coronary disease, a kidney disease, a cardiovascular disease (e.g., coronary artery disease), diabetes, or a liver disease. Distinguishing between different stages of the disease can comprise distinguishing between two stages of a cancer (e.g., stage 0 vs stage 1 or stage 1 vs stage 3).

### Apparatus

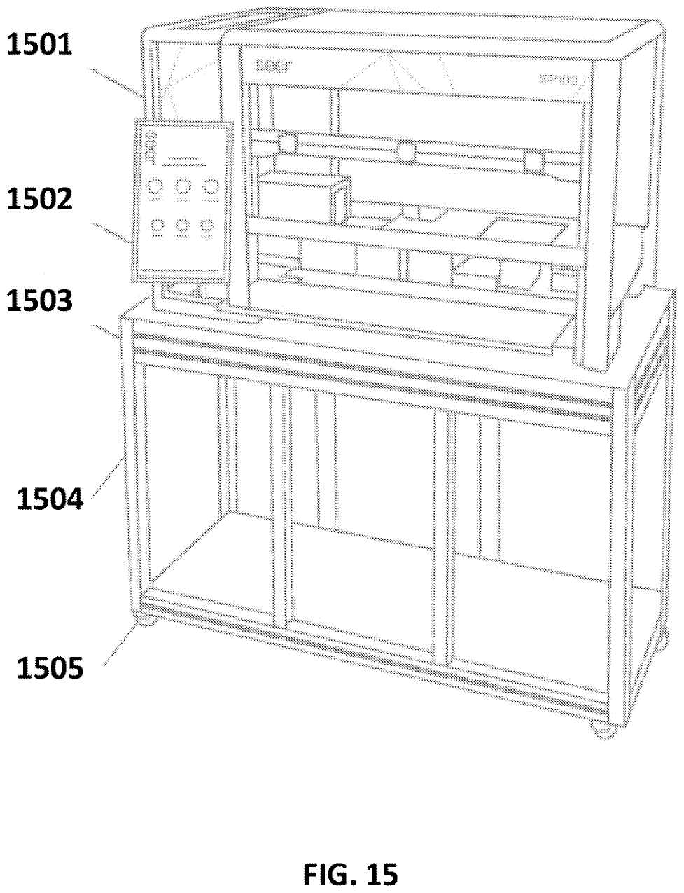

In some aspects, the present disclosure describes an apparatus for assaying a biological sample. FIG. 15 illustrates an apparatus in accordance with some embodiments. In some cases, the apparatus may comprise a stage (1503). In some cases, components of the apparatus may be coupled to the stage. In some cases, the stage may comprise one or more supports (1504) coupled thereto. In some cases, the one or more supports may be configured to move the apparatus (1505). In some cases, components of the apparatus may be coupled to the one or more supports. In some cases, the apparatus may comprise a housing (1501). In some cases, components of the apparatus may be disposed inside the housing. In some cases, the apparatus may comprise a display (1502) coupled thereto. In some cases, housing may comprise one or more supports coupled thereto. In some cases, the apparatus may comprise a rail. In some cases, a component of the apparatus may be movably coupled to the rail.

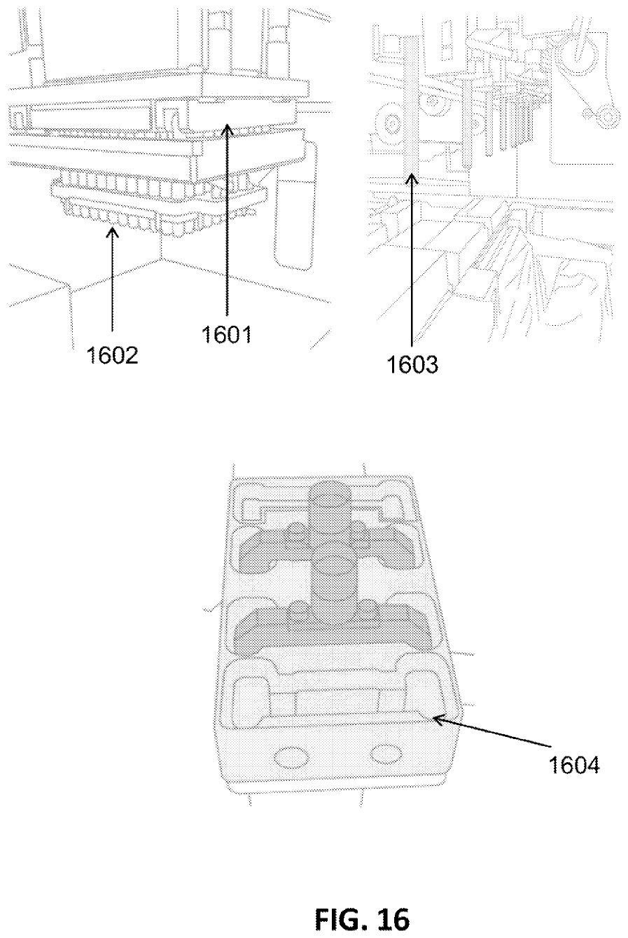

In some cases, the apparatus may comprise one or more transfer units (1601, 1603). In some cases, a transfer unit may be configured to transport liquid samples. FIG. 16 shows transfer units, in accordance with some embodiments. In some cases, a transfer unit may be configured to transport solid samples. In some cases, a transfer unit may comprise a pipette (1602). In some cases, a transfer unit may comprise a plurality of pipettes. In some cases, a transfer unit may comprise a pump. In some cases, a transfer unit may be movable. In some cases, a transfer unit may comprise a rail. In some cases, a transfer unit may comprise a motor configured to move the transfer unit across the rail. In some cases, a transfer unit may comprise a plurality of rails, such that the transfer unit is movable in at least two dimensions. In some cases, a transfer unit may comprise a plurality of rails, such that the transfer unit is movable in at least three dimensions. In some cases, the transfer unit may comprise a robotic arm. In some cases, a transfer unit may comprise a gripper (1604). In some cases, a gripper may be configured to transfer one or more partitions.

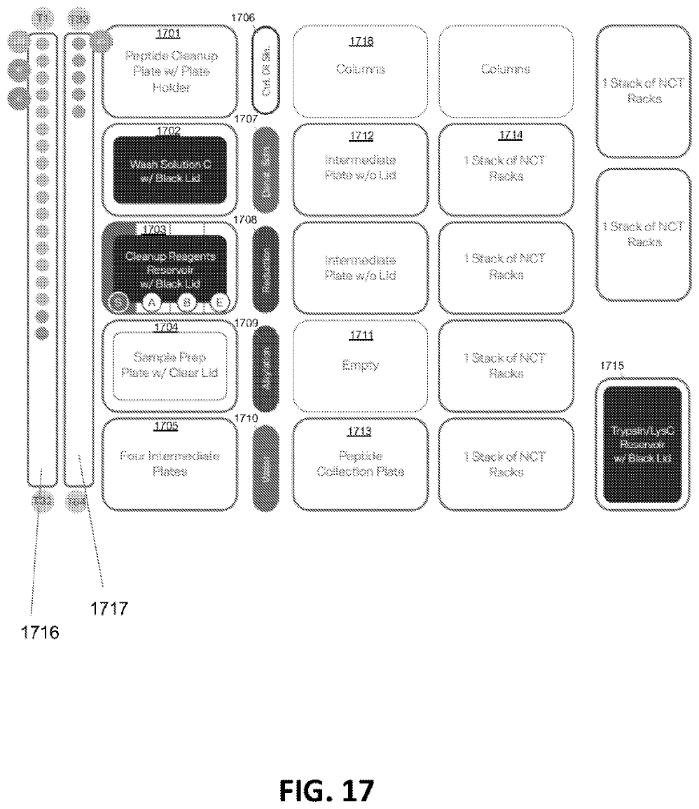

FIG. 17 shows a layout of apparatus components, in accordance with some embodiments. In some cases, the apparatus may comprise a sample storage chamber or well (1716) configured to receive and retain the biological sample. In some cases, the sample storage chamber or well may be configured to receive and retain at least 1, 2, 4, 8, 16, 32, 64, 96, 128, or 256 distinct biological samples. In some cases, the apparatus may comprise a particle storage chamber or well (1717) configured to receive and retain one or more particles. In some cases, the particle storage chamber or well may be configured to receive and retain at least 1, 2, 4, 8, 16, 32, 64, 96, 128, or 256 distinct particles. In some cases, the apparatus may comprise a plurality of plates. In some cases, the apparatus may comprise a cleanup plate (1701), a sample preparation plate (1704), intermediate plate (1705), peptide collection plate (1713), or any combination thereof. In some cases, the apparatus may comprise a plurality of reagent storage chambers or wells. In some cases, the apparatus may comprise a reagent storage chamber or well for a wash solution (1702), cleanup reagents (1703), control dilution solution (1706), denaturation reagent (1707), reduction reagent (1708), alkylation reagent (1709), water (1710), trypsin reagent/lyse reagent (1715), or any combination thereof. In some cases, the apparatus may comprise empty slots (1711) for additional components. In some cases, the apparatus may comprise a rack for pipette tips (1714). In some cases, the apparatus may comprise one or more columns (1718).

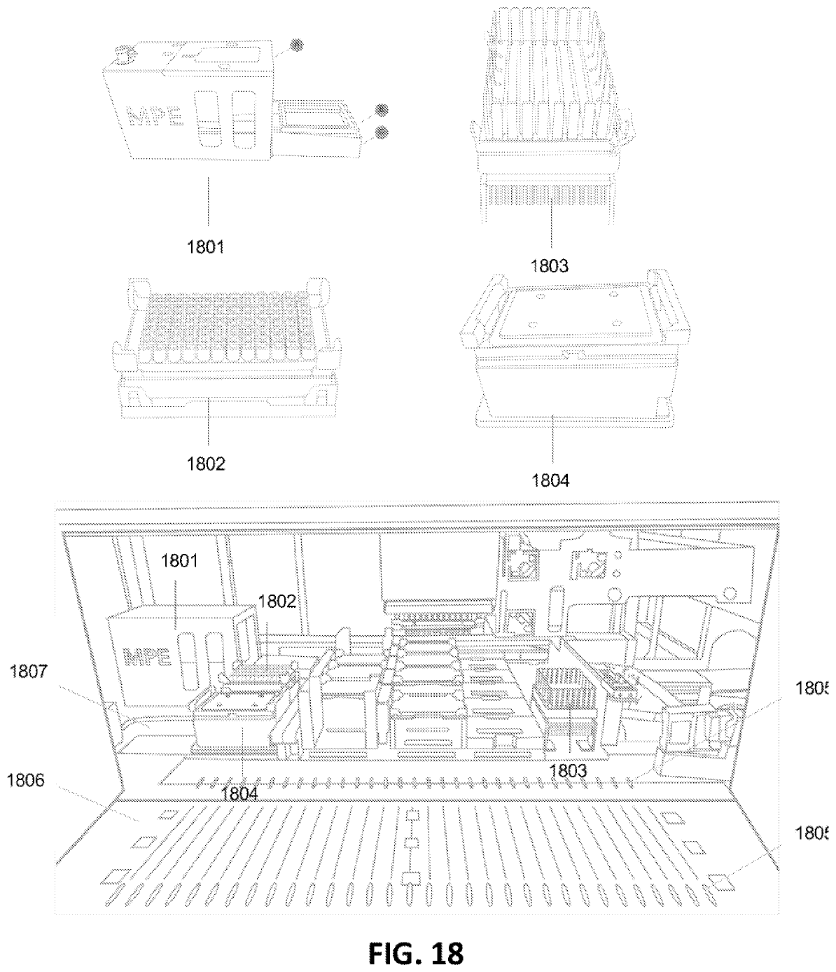

FIG. 18 shows an illustration of apparatus components. In some cases, the apparatus may comprise a filtration system (1801). In some cases, the filtration system may comprise a vacuum. In some cases, the filtration system may comprise a pump. In some cases, the apparatus may comprise a magnetic separation system (1802). In some cases, the magnetic separation system may comprise a magnet. In some cases, the magnetic separation system may be configured to couple with one or more partitions. In some cases, the apparatus may comprise a chiller (1803). In some cases, the apparatus may comprise a heater, shaker, or both (1804). In some cases, the apparatus may comprise rules (1805). In some cases, the apparatus may comprise a working surface (1806). In some cases, the apparatus may comprise a work deck (1807).

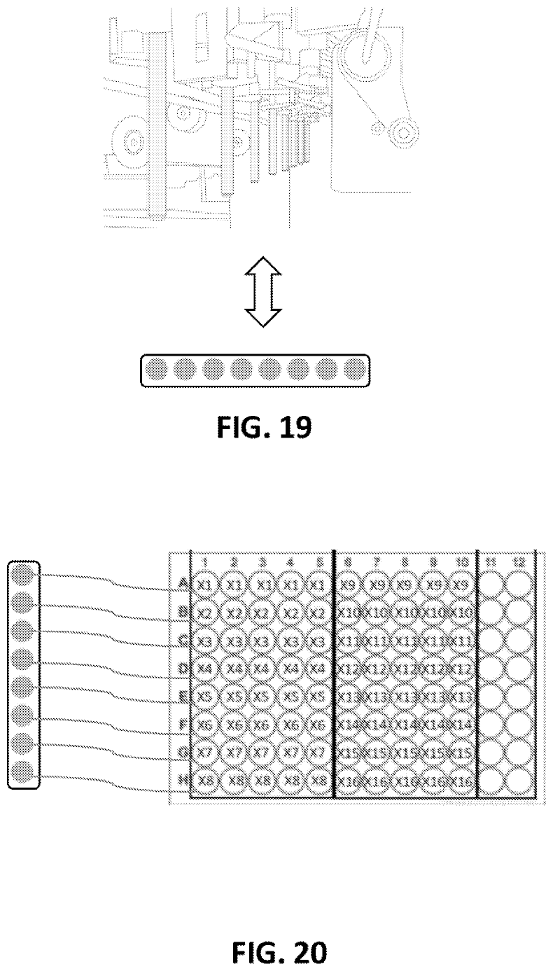

In some cases, the sample storage unit may be operably coupled to the one or more transfer units. In some cases, the one or more transfer units may couple temporarily to the sample storage unit to transport a portion of a biological sample from the sample storage unit. In some cases, the one or more transfer units may move in proximity to the sample storage unit, make contact with a biological sample in the sample storage unit, collect a portion of the biological sample from the sample storage unit, and then move away from the sample storage unit, for example as shown in FIG. 19.

In some cases, the one or more transfer units may be coupled to the sample storage unit via fluidic connection, for example as shown in FIG. 20. In some cases, the one or more transport units may actuate a pump such that a portion of a biological sample in the sample storage unit is transported from the sample storage unit to another component in the apparatus.

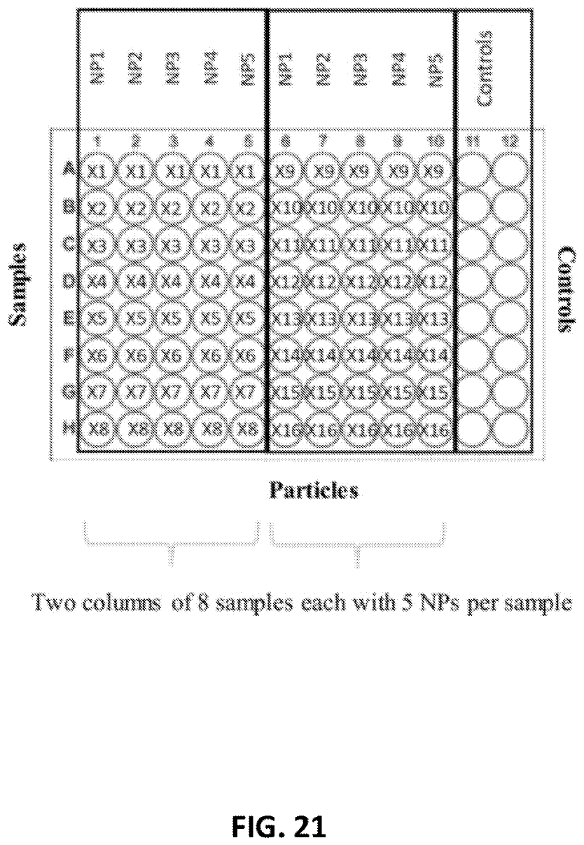

In some cases, the apparatus may comprise a partition containing therein a particle. FIG. 21 shows a plurality of partitions, in accordance with some embodiments. In some cases, the apparatus may comprise a single partition (e.g., an Eppendorf tube) for holding a volume of sample or reagents. In some cases, the apparatus may comprise a plurality of partitions (e.g., a 16 well plate, a 96 well plate, a 384 well plate, a plurality of wells in a microwell plate) for holding sample or reagent volumes. In some cases, a partition may comprise a well, a channel (e.g., a microfluidic channel in a microfluidic device), or a compartment. In some cases, a partition may comprise plasticware (e.g., a plastic multi-well plate), a metal structure (e.g., a metal multi-well plate), a carbon material structure (e.g., a carbon composite material multi-well plate), a gel, glassware, or any combination thereof. In some cases, a fluidic channel or chamber may be a microfluidic or nanofluidic channel or chamber. In some cases, a partition may be sealed (e.g., with a operable plastic slip or a pierceable septum) or be sealable (e.g., may comprise a reusable cap or lid).

In some cases, a partition may be configured to hold a volume of at least 1 to 10 microliters (μl), at least 5 to 25 μl, at least 20 to 50 μl, at least 40 to 200 μl, at least 100 to 500 μl, at least 200 μl to 1 ml, at least 2 ml, at least 3 ml, or more. In some cases, a partition may be configured to hold a volume of less than about 240 μl, 200 μl, 150 μl, 100 μl, 75 μl, 50 μl, 25 μl, 10 μl, 5 μl, 1 μl, or less. In some cases, a partition may be temperature controlled. In some cases, a partition may be configured to prevent or diminish evaporation. In some cases, a partition may be designed to minimize the influx of ambient light.

In some cases, a plurality of partitions may be grouped by particles, samples, control or any combination thereof, as shown in FIG. 21. In this example, the plurality of partitions comprises 8 rows and 12 columns that can be used with 5 types of particles (i.e., NP1, NP2, NP3, NP4, and NP5). In some cases, each nanoparticle may occupy two columns, and up to 16 biological samples may be deposited. In this example, each biological sample is labeled as X1, X2, X3, and so forth, until X16. In some cases, there may be two columns for control experiments, wherein each control well in the columns may receive a control particle composition, a control biological sample, or both. In some cases, each control well may be utilized at a certain step or between steps of an experiment so that an experimental procedure being followed can be troubleshooted. In some cases, particles may be populated in the partitions and then the biological samples may be added in after. In some cases, the biological samples may be populated in the partitions and then the particles may be added in after.

In some cases, a subset of the partitions may be grouped by particle or grouped by sample. In some cases, the plurality of partitions may comprise rows for samples and columns for particles. In some cases, the plurality of partitions may be grouped by a specific composition of particles.

In some cases, a partition may comprise a single particle for a single biological sample. In some cases, a partition may comprise a plurality of particles for a single biological sample. In some cases, a partition may comprise a single particle for a plurality of biological samples. In some cases, a partition may comprise a plurality of particles for a plurality of biological samples.

In some cases, the one or more transfer units may be configured to transport a sample to a single partition, or to divide the sample among a plurality of partitions, or to sequentially transfer the sample from one partition to another. For example, a 5 ml sample may be evenly divided between 500 partitions, resulting in separate 10 μl sample volumes. In some cases, a sample may be mixed with reagents within a partition. In some cases, a sample may undergo a dilution (e.g., with buffer) within a partition.

In some cases, the apparatus may comprise a magnet configured to apply a magnetic field to the contents of a partition, as shown in FIG. 13B. In some cases, the applied magnetic field may separate magnetic substances from non-magnetic substances within a partition. In some cases, the apparatus may comprise a shaker. In some cases, the substrate may be shaken, vibrated, or sonicated by an instrument, as shown in FIG. 13B.

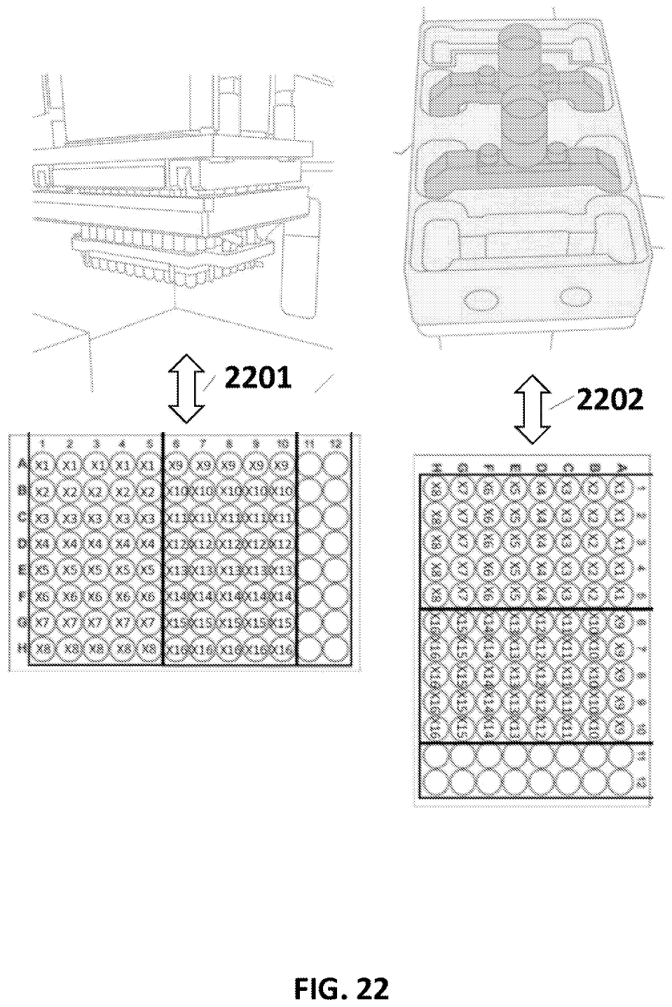

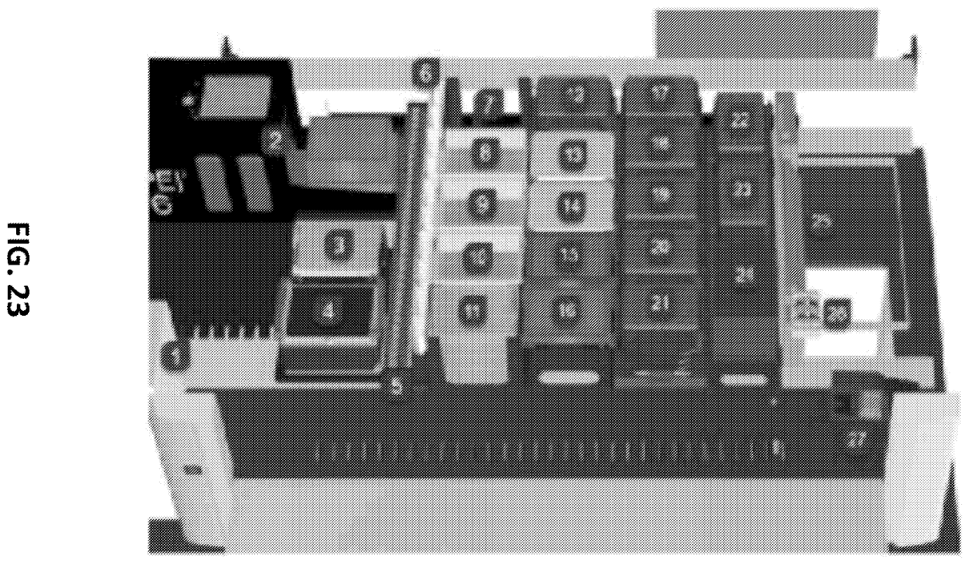

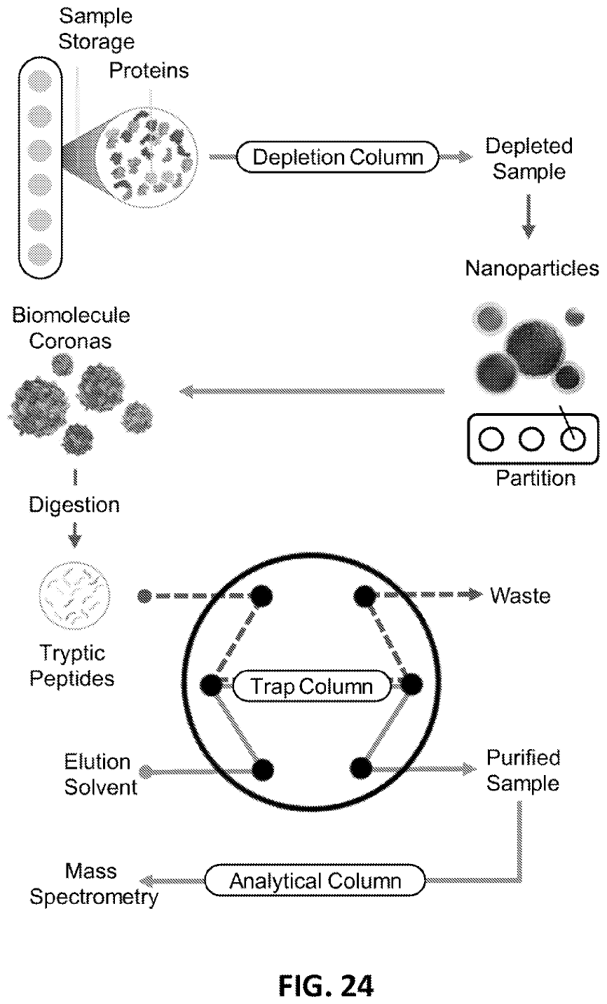

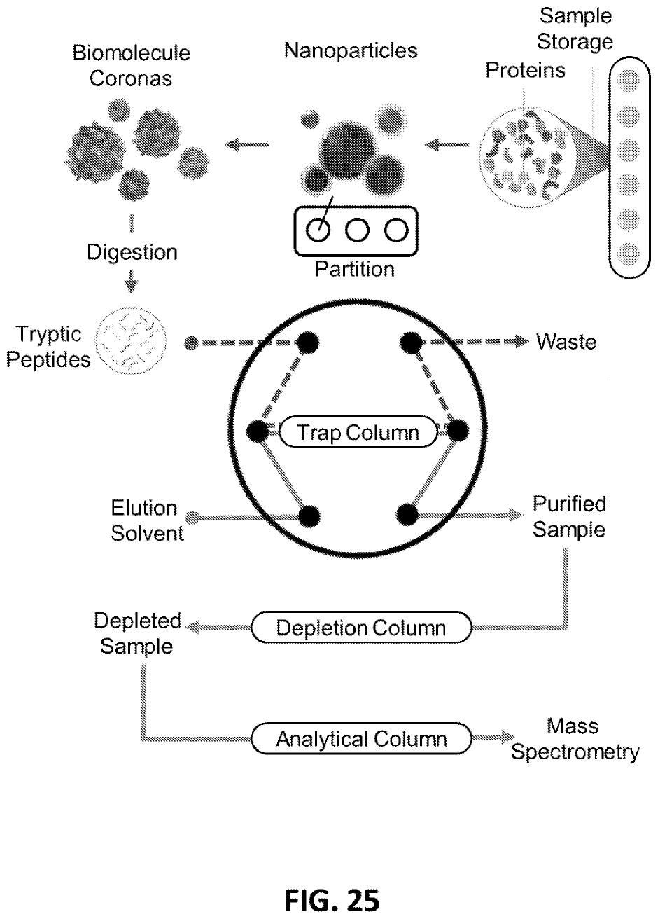

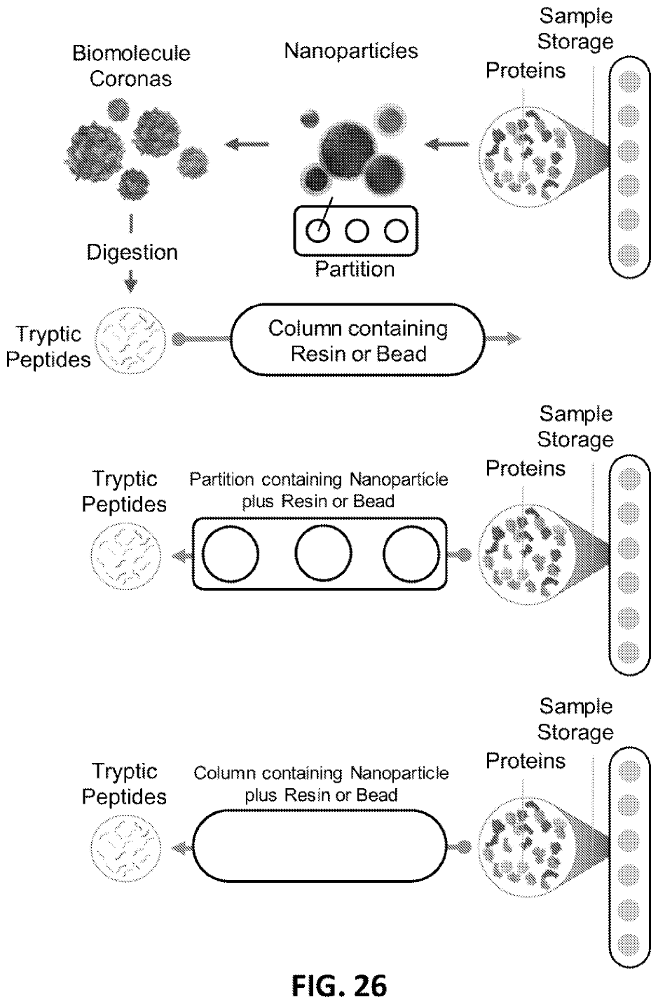

In some cases, the partition may be operably coupled to the one or more transfer units, for example, as shown in FIG. 22. In some cases, the one or more transfer units may couple temporarily to the partition to transport a portion of a biological sample from the partition (2201). In some cases, the one or more transfer units may move in proximity to the partition, make contact with a biological sample in the partition, collect a portion of the biological sample from the partition, and then move away from the partition. In some cases, the one or more transfer units may couple temporarily to the partition to transport the partition (2202). In some cases, the one or more transfer units may move in proximity to the partition, couple to the partition, and transfer the partition.

In some cases, the one or more transfer units may be coupled to the partition via fluidic connection. In some cases, the one or more transport units may actuate a pump such that a portion of a biological sample in the partition is transported from the partition to another component in the apparatus.

In some cases, the apparatus may comprise a plurality of columns. In some cases, a column may comprise a chamber comprising a particulate, for example, a resin or a bead. In some cases, the particulate may comprise a substance configured to bind proteins from a sample. In some cases, the particulate may comprise a substance configured to selectively bind one or more proteins. In some cases, the particulate may comprise a substance configured to selectively bind high abundance proteins. In some cases, the particulate may comprise substance configured to bind one or more proteins under one condition, and release the one or more proteins in another condition. For example, the substance may bind the one or more proteins when using one solvent, and the substance may release the one or more proteins when using another solvent.

In some cases, a column may comprise one or more openings configured to receive and/or output samples and/or solvents. In some cases, a column may comprise sufficient tubing, channels, seals, closures, lids, covers, and etc. in operable communication with other components in the apparatus. In some cases, a column may be in fluidic communication with another column.

In some cases, the apparatus may comprise a trap column. In some cases, the trap column may be configurable between at least two modes. In a first mode, the trap column may be configured to receive a biological sample under conditions sufficient to trap proteins in the biological sample in the trap column. In a second mode, the trap column may be configured to receive a solvent under conditions sufficient to release proteins in the trap column. In some cases, the trap column may comprise dimension sufficient for the substance to trap proteins in the biological sample in less than about 1, 2, 3, 4, 5, 10, 15, 20, 25, 30, 35, 40, 45, 50, 55, or 60 minutes. In some cases, the trap column may comprise dimension sufficient for the substance to trap proteins in the biological sample in less than about 1, 2, 3, 4, 5, 6, 7, 8, 9, or 10 hours. In some cases, the trap column may comprise dimension sufficient for the substance to release proteins in the biological sample in less than about 1, 2, 3, 4, 5, 10, 15, 20, 25, 30, 35, 40, 45, 50, 55, or 60 minutes. In some cases, the trap column may comprise dimension sufficient for the substance to release proteins in the biological sample in less than about 1, 2, 3, 4, 5, 6, 7, 8, 9, or 10 hours.

In some cases, the apparatus may comprise a depletion column. In some cases, a depletion column may comprise a substance configured to selectively bind high abundance proteins. In some cases, the substance may selectively bind to at least 1, 2, 3, 4, 5, 6, 7, 8, 9, 10, 11, 12, 13, 14, 15, 16, 17, 18, 19, 20, 30, 40, 50, 60, 70, 80, 90, or 100 high abundance proteins. In some cases, the substance may selectively bind to one or more high abundance proteins selected from the group consisting of: albumin, IgG, IgA, IgM, IgD, IgE, IgG (light chains), alpha-1-acid glycoprotein, fibrinogen, haptoglobin, alpha-1-antitrypsin, alpha-2-macroglobulin, transferrin, and apolipoprotein A-1. In some cases, the depletion column may comprise dimension sufficient for the substance to selectively bind high abundance proteins in the biological sample in less than about 1, 2, 3, 4, 5, 10, 15, 20, 25, 30, 35, 40, 45, 50, 55, or 60 minutes. In some cases, the depletion column may comprise dimension sufficient for the substance to selectively bind high abundance proteins in the biological sample in less than about 1, 2, 3, 4, 5, 6, 7, 8, 9, or 10 hours.

In some cases, the apparatus may comprise an analytical column. In some cases, an analytical column may comprise a substance configured to chromatographically separate proteins. In some cases, the analytical column may comprise dimension sufficient for the substance to chromatographically separate proteins in the biological sample in less than about 1, 2, 3, 4, 5, 10, 15, 20, 25, 30, 35, 40, 45, 50, 55, or 60 minutes. In some cases, the analytical column may comprise dimension sufficient for the substance to chromatographically separate proteins in the biological sample in less than about 1, 2, 3, 4, 5, 6, 7, 8, 9, or 10 hours.

In some cases, the plurality of columns may be operably coupled to the one or more transfer units. In some cases, the one or more transfer units may couple temporarily to the plurality of columns to transport a portion of a biological sample from the plurality of columns. In some cases, the one or more transfer units may move in proximity to the plurality of columns, make contact with a biological sample in the plurality of columns, collect a portion of the biological sample from the plurality of columns, and then move away from the plurality of columns.

In some cases, the one or more transfer units may be coupled to the plurality of columns via fluidic connection. In some cases, the one or more transport units may actuate a pump such that a portion of a biological sample in the plurality of columns is transported from the plurality of columns to another component in the apparatus. In some cases, the one or more transport units may transport the portion of the biological sample between columns in the plurality of columns.

In some cases, the apparatus may comprise a control unit comprising one or more processors. In some cases, the control unit may be in electrical communication with the one or more transfer units. In some cases, the control unit may comprise instructions that when executed actuates the one or more transfer units to transfer a sample between components in the apparatus. In some cases, the one or more transfer units may be configured to transfer the biological sample from the sample storage unit to the depletion column to produce a depleted sample. In some cases, the one or more transfer units may be configured to transfer the depleted sample to the partition to adsorb a plurality of biomolecules from the biological sample onto the particle. In some cases, the one or more transfer units may be configured to transfer the plurality of biomolecules to the trap column to produce a purified sample. In some cases, the one or more transfer units may be configured to transfer the purified sample to the analytical column to produce a separated sample. In some cases, the one or more transfer units may be configured to transfer the separated sample to a mass spectrometer for mass spectrometry analysis on the plurality of biomolecules to produce a plurality of signals. In some cases, the one or more transfer units may be configured to transfer the biological sample from the sample storage unit to adsorb a plurality of biomolecules from the biological sample onto the particle. In some cases, the one or more transfer units may be configured to transfer the plurality of biomolecules to the trap column to produce a purified sample. In some cases, the one or more transfer units may be configured to transfer the purified sample to the depletion column to produce a depleted sample. In some cases, the one or more transfer units may be configured to transfer the depleted sample to the analytical column to produce a separated sample. In some cases, the one or more transfer units may be configured to transfer the separated sample to a mass spectrometer for mass spectrometry analysis on the plurality of biomolecules to produce a plurality of signals.

In some cases, the apparatus may comprise a plurality of reagent storage units. In some cases, the plurality of reagent storage units may comprise a reagent storage unit containing therein an aqueous solvent. In some cases, the plurality of reagent storage units may comprise a reagent storage unit containing therein an elution solvent. In some cases, the plurality of reagent storage units may comprise a waste storage unit containing therein waste. In some cases, the plurality of reagent storage units may comprise a wash solution unit containing therein a wash solution. In some cases, the plurality of reagent storage units may comprise an enzyme reagent unit, containing therein enzymes for processing a biological sample.

In some cases, the apparatus may comprise a chiller. In some cases, the apparatus may comprise a heater. In some cases, the apparatus may comprise a filter plate. In some cases, the apparatus may comprise an evaporator. In some cases, the apparatus may comprise a vacuum. In some cases, the apparatus may comprise a sample preparation unit comprising an HPLC column. In some cases, the apparatus may comprise a sample preparation unit comprising a filter. In some cases, the apparatus may comprise a sample preparation unit comprising a centrifuge. In some cases, the one or more transfer units may be operably coupled to the sample preparation unit.

FIG. 23 shows some components of an apparatus, in accordance with some embodiments. TABLE 1 provides a description of the components in FIG. 23.

TABLE 1
Modules for the Automated system

| Number | Description |
|---|---|
| 1 | CO-RE 96 Probe Head |
| 2 | MPE² Filter |
| 3 | Magnet Position |
| 4 | HHS |
| 5 | Magnet Position |
| 6 | Nanoparticle & Plasma Samples Tubes |
| 7 | Plate-Stack Module |
| 8 | TE Buffer Reservoir |
| 9 | Wetting Reagent (100% Methanol) Reservoir |
| 10 | Condition Reagent (H₂O) |
| 11 | Plate Stack Module |
| 12 | NTR Module |
| 13 | Lid Park Position |
| 14 | Lid Park Position |
| 15 | Plate Carrier Position |
| 16 | Plate Carrier Position |
| 17 | Nested Tip Rack (NTR) Stack Module |
| | for Multi-Probe Head (MPH) |
| 18 | NTR Stack Module for MPH |
| 19 | NTR Stack Module for MPH |
| 20 | NTR Stack Module for MPH |
| 21 | NTR Stack Module for MPH |
| 22 | NTR Stack Module for Channels |
| 23 | NTR Stack Module for Channels |
| 24 | Inheco Cold Plate Air Cooled (CPAC) |
| 25 | Tip Waste |
| 26 | Compressed O-Ring Expanion (CO-RE) Paddles |
| 27 | Autoload |
| 28 | STARlet Chassis |

In some cases, the one or more transfer units is operably coupled to the plurality of reagent storage units. In some cases, the one or more transfer units may couple temporarily to the plurality of reagent storage units to transport a reagent from the plurality of reagent units to another component in the apparatus. In some cases, the one or more transfer units may move in proximity to the plurality of reagent storage units, make contact with a reagent in the plurality of reagent storage units, collect a reagent from the plurality of reagent storage units, and then move away from the plurality of reagent storage units.

In some cases, the one or more transfer units may be coupled to the plurality of reagent storage units via fluidic connection. In some cases, the one or more transport units may actuate a pump such that a reagent in the plurality of reagent storage units is transported from the plurality of reagent storage units to another component in the apparatus.

### Computer Control Systems

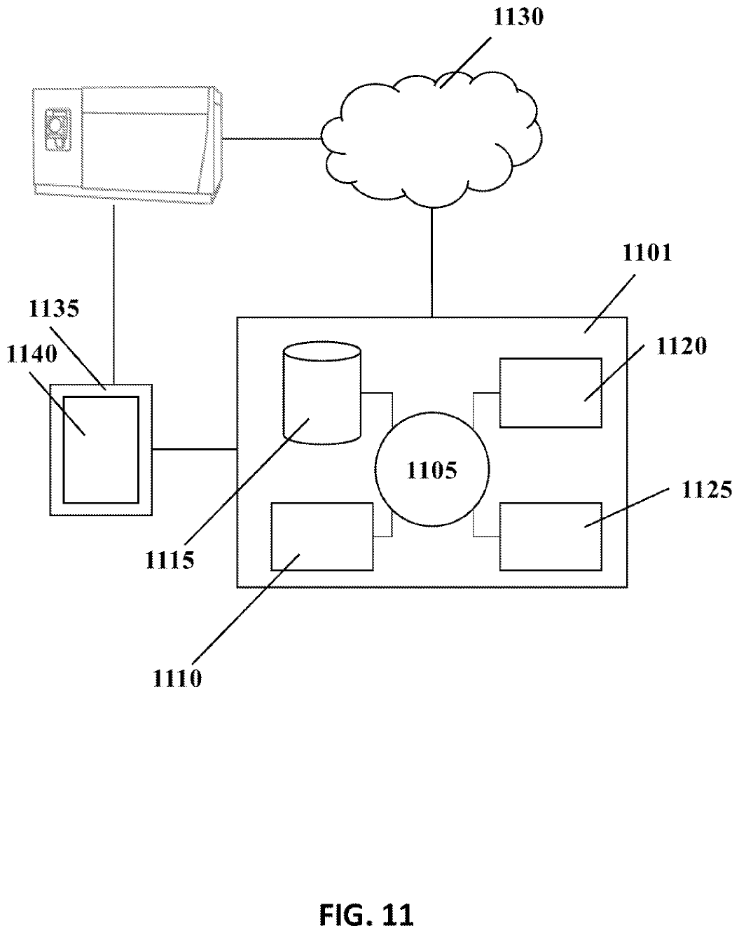

The present disclosure provides computer control systems that are programmed to implement methods of the disclosure. FIG. 11 shows a computer system that is programmed or otherwise configured to implement methods provided herein. The computer system 1101 can regulate various aspects of the assays disclosed herein, which are capable of being automated (e.g., movement of any of the reagents disclosed herein on a substrate). The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example a readout of the proteins identified using the methods disclosed herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105.

Determination, analysis or statistical classification is done by methods known in the art, including, but not limited to, for example, a wide variety of supervised and unsupervised data analysis and clustering approaches such as hierarchical cluster analysis (HCA), principal component analysis (PCA), Partial least squares Discriminant Analysis (PLSDA), machine learning (also known as random forest), logistic regression, decision trees, support vector machine (SVM), k-nearest neighbors, naive bayes, linear regression, polynomial regression, SVM for regression, K-means clustering, and hidden Markov models, among others. The computer system can perform various aspects of analyzing the protein sets or protein corona of the present disclosure, such as, for example, comparing/analyzing the biomolecule corona of several samples to determine with statistical significance what patterns are common between the individual biomolecule coronas to determine a protein set that is associated with the biological state. The computer system can be used to develop classifiers to detect and discriminate different protein sets or protein corona (e.g., characteristic of the composition of a protein corona). Data collected from the presently disclosed sensor array can be used to train a machine learning algorithm, specifically an algorithm that receives array measurements from a patient and outputs specific biomolecule corona compositions from each patient. Before training the algorithm, raw data from the array can be first denoised to reduce variability in individual variables.

Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FPgrowth algorithm; Hierarchical clustering, such as Singlelinkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory. A computer system may be adapted to implement a method described herein. The system includes a central computer server that is programmed to implement the methods described herein. The server includes a central processing unit (CPU, also “processor”) which can be a single core processor, a multi core processor, or plurality of processors for parallel processing. The server also includes memory (e.g., random access memory, read-only memory, flash memory); electronic storage unit (e.g. hard disk); communications interface (e.g., network adaptor) for communicating with one or more other systems; and peripheral devices which may include cache, other memory, data storage, and/or electronic display adaptors. The memory, storage unit, interface, and peripheral devices are in communication with the processor through a communications bus (solid lines), such as a motherboard. The storage unit can be a data storage unit for storing data. The server is operatively coupled to a computer network (“network”) with the aid of the communications interface. The network can be the Internet, an intranet and/or an extranet, an intranet and/or extranet that is in communication with the Internet, a telecommunication or data network. The network in some cases, with the aid of the server, can implement a peer-to-peer network, which may enable devices coupled to the server to behave as a client or a server.

The storage unit can store files, such as subject reports, and/or communications with the data about individuals, or any aspect of data associated with the present disclosure.

The computer server can communicate with one or more remote computer systems through the network. The one or more remote computer systems may be, for example, personal computers, laptops, tablets, telephones, Smart phones, or personal digital assistants.

In some applications the computer system includes a single server. In other situations, the system includes multiple servers in communication with one another through an intranet, extranet and/or the internet.

The server can be adapted to store measurement data or a database as provided herein, patient information from the subject, such as, for example, medical history, family history, demographic data and/or other clinical or personal information of potential relevance to a particular application. Such information can be stored on the storage unit or the server and such data can be transmitted through a network.

Methods as described herein can be implemented by way of machine (or computer processor) executable code (or software) stored on an electronic storage location of the server, such as, for example, on the memory, or electronic storage unit. During use, the code can be executed by the processor. In some cases, the code can be retrieved from the storage unit and stored on the memory for ready access by the processor. In some situations, the electronic storage unit can be precluded, and machine-executable instructions are stored on memory. Alternatively, the code can be executed on a second computer system.

Aspects of the systems and methods provided herein, such as the server, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

The computer systems described herein may comprise computer-executable code for performing any of the algorithms or algorithms-based methods described herein. In some applications the algorithms described herein will make use of a memory unit that is comprised of at least one database.

Data relating to the present disclosure can be transmitted over a network or connections for reception and/or review by a receiver. The receiver can be but is not limited to the subject to whom the report pertains; or to a caregiver thereof, e.g., a health care provider, manager, other health care professional, or other caretaker; a person or entity that performed and/or ordered the analysis. The receiver can also be a local or remote system for storing such reports (e.g. servers or other systems of a “cloud computing” architecture). In one embodiment, a computer-readable medium includes a medium suitable for transmission of a result of an analysis of a biological sample using the methods described herein.

Aspects of the systems and methods provided herein can be embodied in programming. Various aspects of the technology may be thought of as “products” or “articles of manufacture” typically in the form of machine (or processor) executable code and/or associated data that is carried on or embodied in a type of machine readable medium. Machineexecutable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide nontransitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### Classification of Protein Corona Using Machine Learning

The method of determining a set of proteins associated with the disease or disorder and/or disease state include the analysis of the corona of the at least two samples. This determination, analysis or statistical classification is done by methods known in the art, including, but not limited to, for example, a wide variety of supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive bayes, linear regression, polynomial regression, SVM for regression, K-means clustering, and hidden Markov models, among others. In other words, the proteins in the corona of each sample are compared/analyzed with each other to determine with statistical significance what patterns are common between the individual corona to determine a set of proteins that is associated with the disease or disorder or disease state.

Generally, machine learning algorithms are used to construct models that accurately assign class labels to examples based on the input features that describe the example. In some case it may be advantageous to employ machine learning and/or deep learning approaches for the methods described herein. For example, machine learning can be used to associate the protein corona with various disease states (e.g. no disease, precursor to a disease, having early or late stage of the disease, etc.). For example, in some cases, one or more machine learning algorithms are employed in connection with a method of the invention to analyze data detected and obtained by the protein corona and sets of proteins derived therefrom. For example, in one embodiment, machine learning can be coupled with the sensor array described herein to determine not only if a subject has a pre-stage of cancer, cancer or does not have or develop cancer, but also to distinguish the type of cancer.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### Examples

The following examples are illustrative and non-limiting to the scope of the compositions, devices, systems, kits, and methods described herein.

### Example 1

### Human Plasma Sample Preparation

This example provides a method for preparing a plasma sample for analysis. In this example, a human plasma sample was subjected to depletion using an Agilent 1260 Infinity II Bioinert HPLC system. Plasma depletion was conducted by first diluting 20 μL of plasma to a final volume of 100 μL with Agilent “Buffer A” plasma depletion mobile-phase. Each diluted sample was filtered through an Agilent 0.22μ cellulose acetate spin filter to remove any particulates and transferred to a 96-well plate incubated at 4° C. 80 μL of the diluted plasma was then injected into an Agilent 4.6×50 mm Human 14 Multiple Affinity Removal System (MARS 14) depletion column held at 20° C., and eluted with 100% “Buffer A” mobile-phase flowing at a rate of 0.125 mL/min. Proteins eluting from the column were detected using the Agilent UV absorbance detector operated at 209 nm with a bandwidth of 4 nm. The early eluting peak for each injection, representing the depleted plasma proteins, was collected using a refrigerated fraction collector with peak-intensity based triggering set to a 200 mAu threshold with a maximum peak width of 3 minutes. After peak collection, the fractions were held at 4° C. The sample volume was then reduced to approximately 20 μL using an Amicon Centrifugal Concentrator (Amicon Ultra-0.5 mL, 3 k MWCO) with a centrifuge operating at 4° C. and 14,000×g. Each depleted sample was then reduced, alkylated, digested, desalted, and analyzed according to the sample preparation and MS analysis protocols described below. During each sample depletion cycle, the MARS 14 column was regenerated with the Agilent “Buffer B” mobile-phase for approximately 4½ minutes at a flow rate of 1 mL/min and equilibrated back to the original protein capture condition by flowing “Buffer A” at 1 mL/min for approximately 9 minutes.

### Example 2

### Identification of Particle Properties Associated with Protein Enrichment

This example covers a method for identifying particle properties associated with the enrichment of different types of proteins. A wide range of particle properties were interrogated to identify sets of properties responsible for the adsorption of particular proteins and protein groups. A total of 37 different nanoparticles were separately incubated with human plasma samples, and the content of the resulting protein coronas associated with the particles were analyzed by liquid chromatography (LC) coupled to tandem mass spectrometry (MS/MS) with data independent acquisition.

Human plasma samples were prepared as described in Example 1 above. Particles were provided in dry powdered form, and reconstituted in DI water to make a final concentration of 5 mg/mL for all NPs except for SP-339-008, SP-353-002 which had a final concentration of 2.5 mg/mL and SP-373-007 with a final concentration of 10 mg/mL, followed by 10 min sonication and vortexing for 2-3 sec. To form the protein corona, 100 μL of NP suspension was mixed with 100 μL of plasma samples in microtiter plates. The plates were sealed and incubated at 37° C. for 1 hour with shaking at 300 rpm. After incubation, the plate was placed on top of a magnetic collection device for 5 mins to draw down the NPs. The supernatant, containing the non-corona unbound proteins was aspirated by pipetting. The protein corona was washed a total of three times with 200 μL of wash buffer which contains 150 mM KCl and 0.05% CHAPS in a Tris EDTA buffer with pH of 7.4.

To digest the proteins bound onto NPs, a trypsin digestion kit (iST 96X, PreOmics, Germany) was used according to protocols provided by the vendor. Briefly, 50 μL of Lyse buffer was added to each well and heated at 95° C. for 10 min with agitation at 1000 rpm. After the plates were cooled to room temperature, trypsin digestion buffer was added, and the plates were incubated at 37° C. for 3 hours with shaking at 500 rpm. After stopping the digestion process by addition of the supplied stop buffer, the NPs were removed from the reaction by magnetic collection, and the remaining reaction supernatant was cleaned up with the supplied filter cartridge (styrenedivinylbenzene reversed-phase sulfonate/SDB-RPS) kit. The peptide was eluted with 75 μL of elution buffer twice and combined. Peptide concentration was measured by a quantitative colorimetric peptide assay kit from Thermo Fisher Scientific (Waltham, MA).

The peptides were subject to LC-MS/MS with data-independent acquisition. For this analysis, the peptides were reconstituted in a solution of 0.1% FA and 3% ACN spiked with 5 fmol/uL PepCalMix from SCIEX (Framingham, MA). Five μg of peptides in 10 uL of reconstitution buffer was used for each constant mass MS injection. Each sample was analyzed by an Eksigent nanoLC system coupled with a SCIEX TripleTOF 6600+ mass spectrometer equipped with an OptiFlow source using a trap-and-elute method. First, the peptides were loaded on a ChromXP C18CL (0.3 mm ID×10 mm) trap column and then separated on a Phenomenex Kinetex analytical column (150 mm×0.3 mm, C18, 2.6 μm, 100 Å) at a flow rate of 5 uL/min using a gradient of 3-32% solvent B (0.1% FA, 100% ACN) mixed into solvent A (0.1% FA, 100% water) over 20 min, resulting in a 33 min total run time. The mass spectrometer was operated in SWATH™ mode using 100 variable windows across the 400-1250 m/z range.

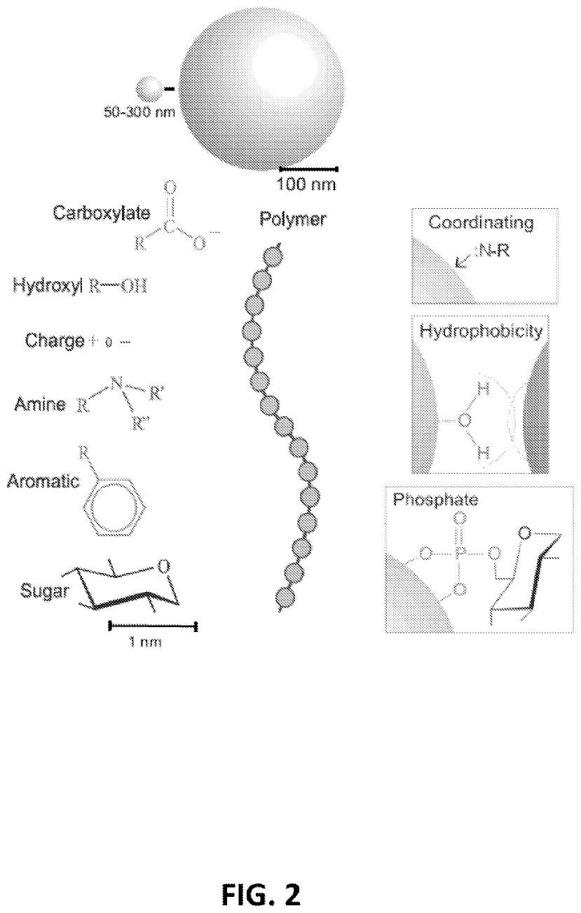

The measured protein corona compositions were interrogated as a function of individual particle properties. Each particle was annotated to describe its functionalization, including charge, hydrophobicity, and the presence of amine, carboxylate, sugar, phosphate, hydroxyl, or aromatic groups FIG. 2. To account for the possibility that a reaction to functionalize a particle may not necessarily result in complete coverage of the particle, particles were further categorized according to their “reaction class,” which is the particular type of chemical reaction used in the last step of the particle's synthesis. FIG. 3A provides protein intensity patterns grouped by particle property.

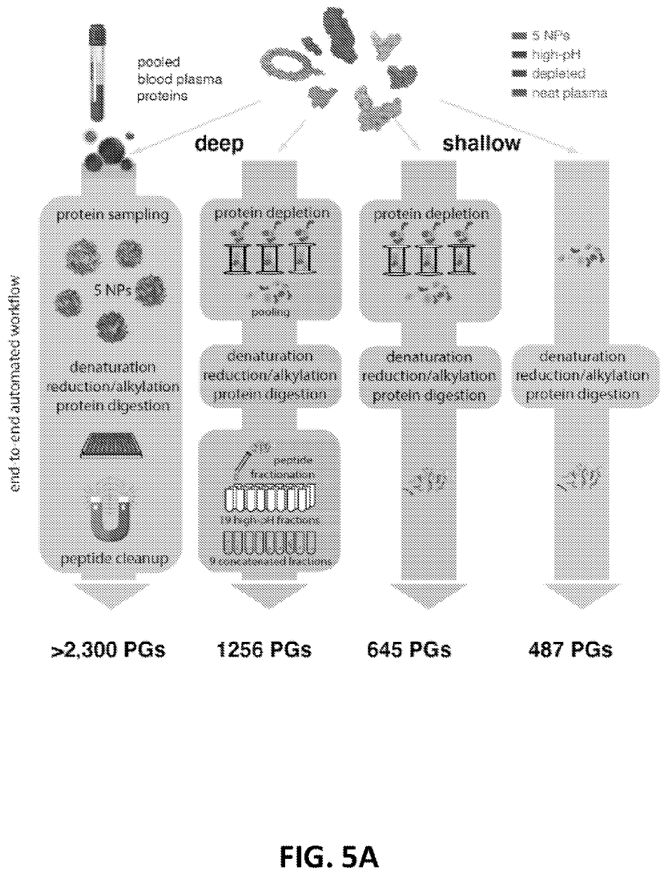

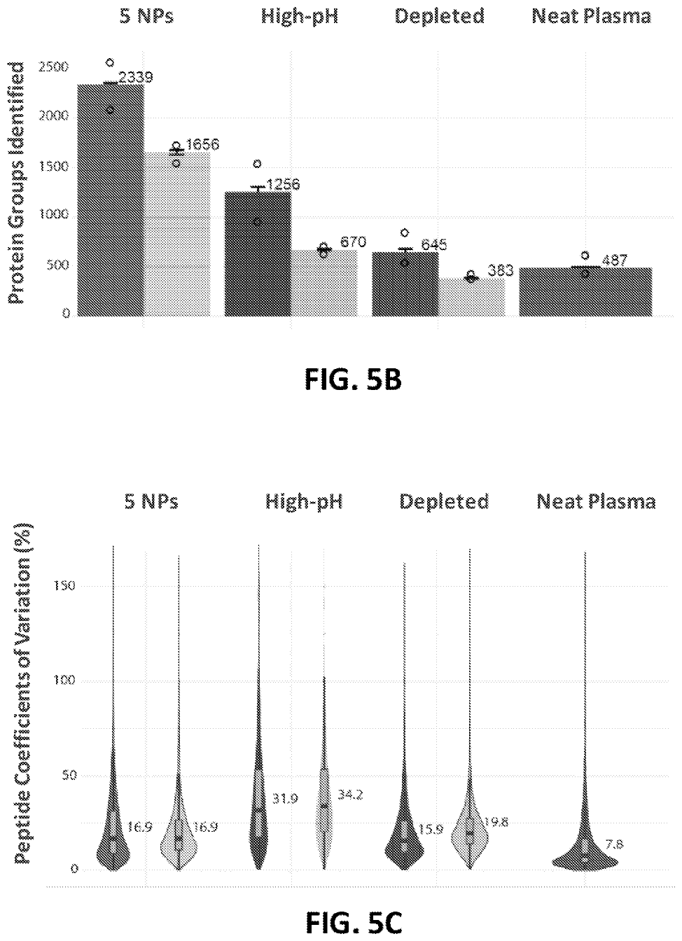

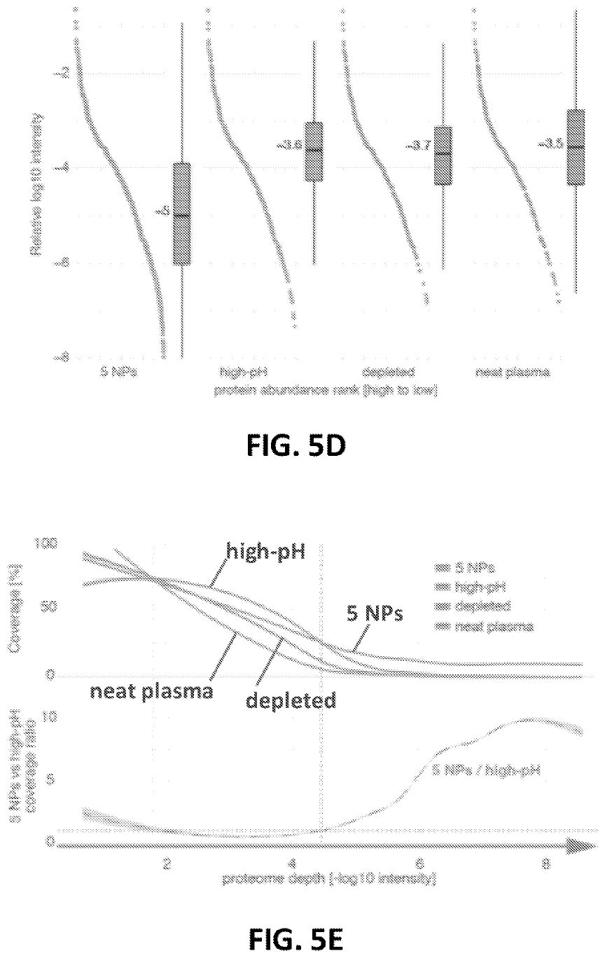

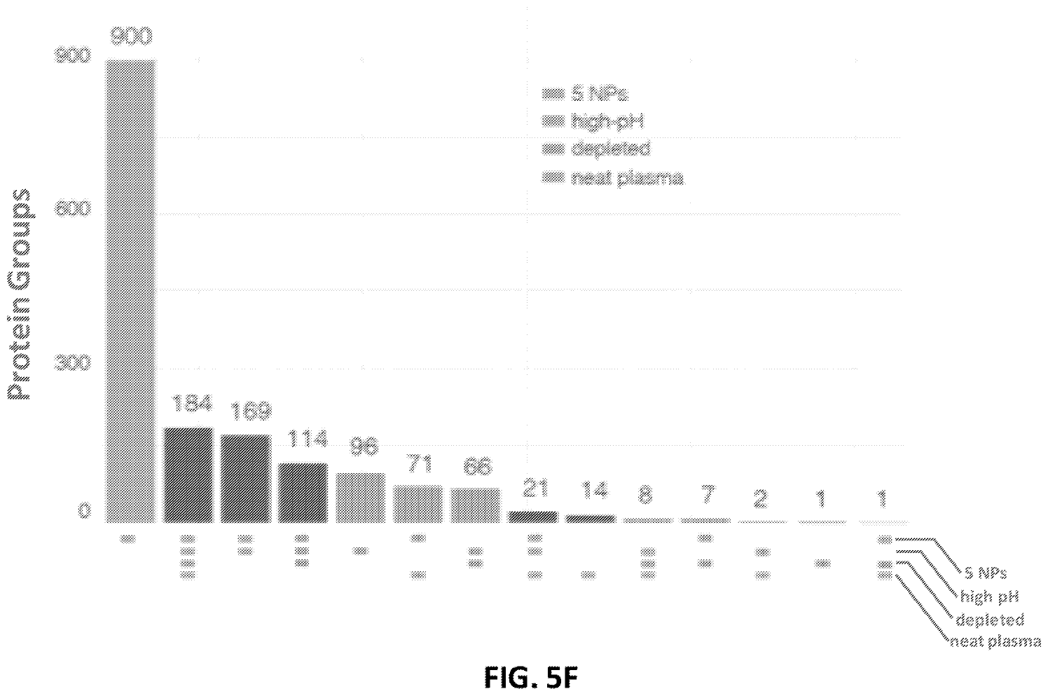

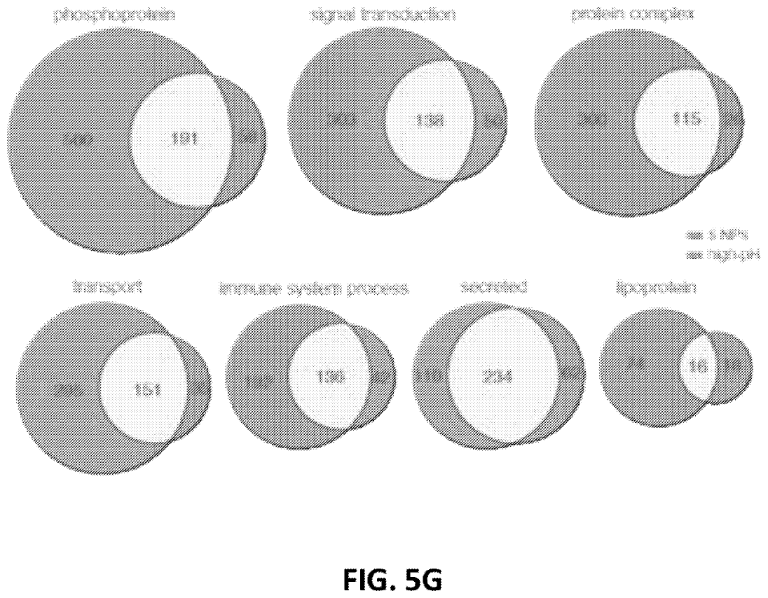

Next, 1D annotation enrichment analysis was used to identify clusters of physicochemical properties with similar effects on protein enrichment. As shown in FIG. 3B, hierarchical clustering of the 37 nanoparticles based on their 1D enrichment scores yielded five conceptually distinct groups of particles. Fisher's exact test was applied to each cluster to highlight their dominant distinguishing properties. Cluster 1 (C1, FIG. 3B) is comprised of “sponge” or silica-coated SPION particles treated with succinic anhydride, which undergoes ring-opening to form surface-exposed carboxylate groups. Several members of this group (S-182 through S-186) had other groups (e.g., butyl, pyridyl, hydroxyethyl) tethered to their surfaces via amide coupling. Cluster 2 (C2, FIG. 3B) includes the core sponge particle (S-113), as well as other particles expected to be hydrophilic or amphiphilic. Two of the particles (P-039, S-179) in this cluster have polystyrene surfaces functionalized with acidic or anionic groups (carboxylic acid and sulfonate, respectively). Cluster 3 (C3, FIG. 3B) consists primarily of amine-functionalized particles, along with a single hydroxyethyl functionalized particle and an isopropylamide particle (with the isopropylamide attached via ARGET-ATRP polymerization). Clusters 4 (C4, FIG. 3B) and 5 (C5, FIG. 3B) consist primarily of particles with hydrophilic surface functionalizations, covering most of the hydroxyl-functionalized particles and all of the sugar-functionalized particles. Clusters 4 and 5 also included several carboxylate-functionalized particles, a methylamine-functionalized particle, and a cationic tetraalkylammonium-functionalized particle.

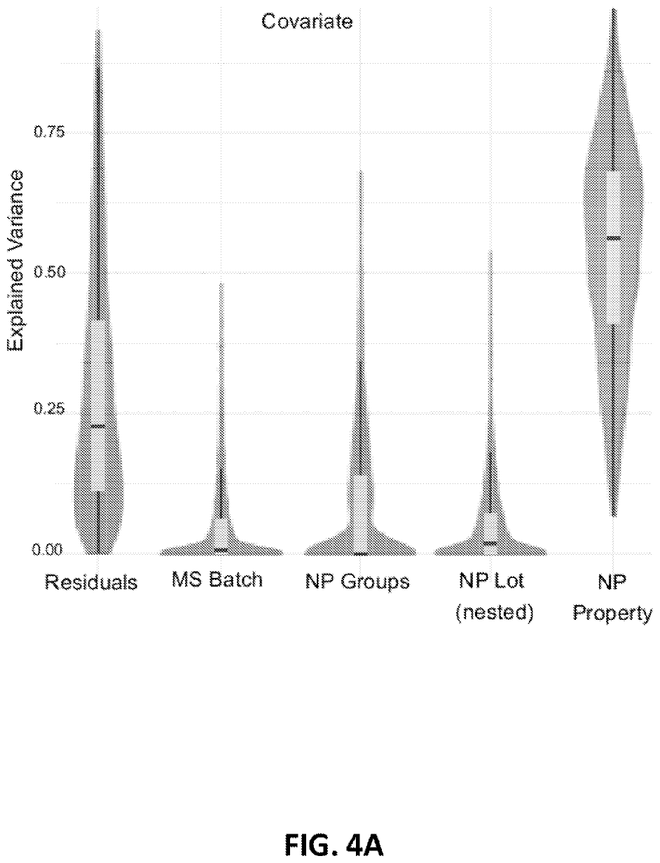

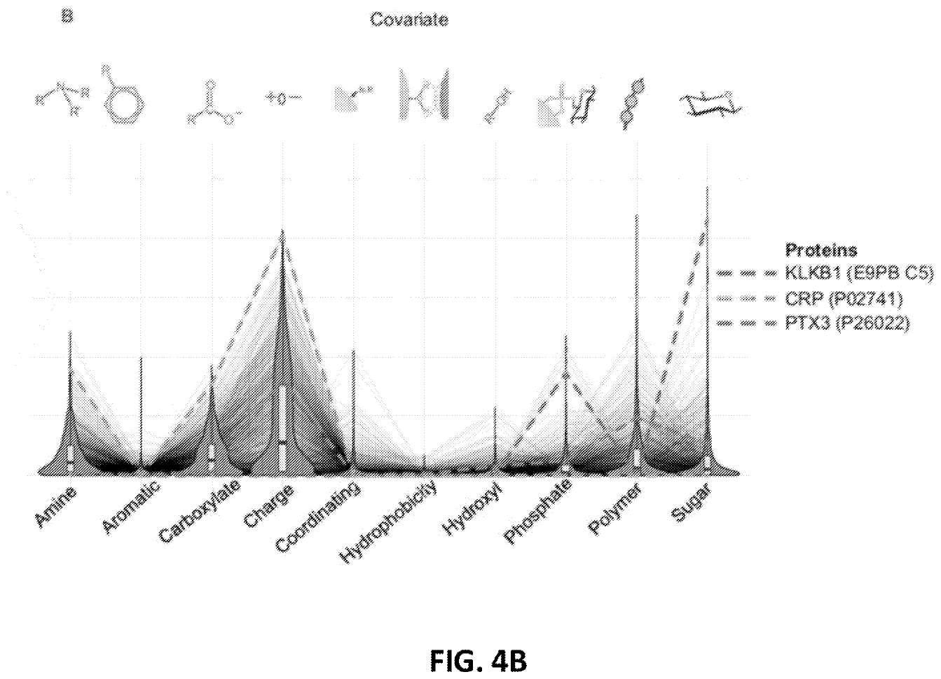

Next, a variance decomposition was performed on the protein corona data to determine how much of the observed variance in protein abundance (approximated by mass spectrometric signal intensities), the results of which are provided in FIG. 4A. Across the 37 particle-types assayed, greater than 50% of the variance in the majority of protein intensities were explained by particle properties. FIG. 4B displays the degrees of explained variance in protein intensities as functions of different particle properties. Among all functional groups, charge and carboxylate provided the largest individual contributions to protein intensity levels.

### Example 3

### Comparison of Particle Panel Enrichment to Plasma Depletion for Protein Identification with Data-Independent Mass Spectrometry

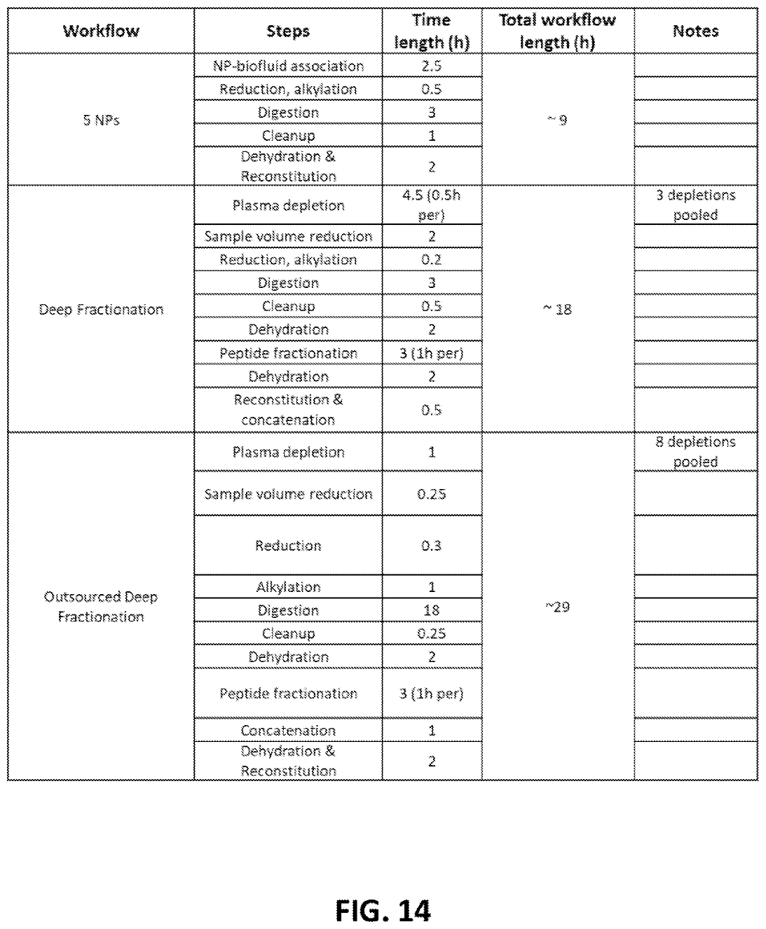

This example compares different plasma proteomic workflows in terms of their depths of coverage. Mass spectrometric analysis was performed on proteins collected on a five particle panel, a proteins from plasma subjected to high-pH depletion (“Deep Fractionation”), depleted plasma, and neat plasma. Plasma sample preparation, particle corona preparation and collection, and mass spectrometric analysis were performed as outlined in Examples 1 & 2. The workflows for each of the four analysis types are summarized in FIG. 5A. Each workflow was performed in triplicate. The workflow time and steps for the five particle panel workflow and for the two Deep Fractionation workflows are provided in FIG. 14.

FIG. 5B summarizes the number of protein groups identified with each workflow. Each bar indicates the median number of protein groups yielded for each workflow. Error bars denote standard deviations of assay replicates. The top dash depicts the number of identified proteins in any of the samples and the lower dash represents the number of identified proteins in 3 out of 3 assay replicates (complete features) The greatest number of proteins were identified by the 5 particle panel workflow, which generated greater than 2300 protein group identifications, which translates to about 2-times, about 4-times, and about 6-times more protein group identifications compared to high-pH depletion, depletion, and neat plasma analysis. FIG. 5C summarizes the variation in peptide intensities over multiple LC-MS/MS parameters, including LC-gradient length and MS instrumentation. Comparing the precision of peptide quantification between different workflows, the five particle panel, plasma depletion, and neat plasma methods resulted in the median coefficients of variation (CV) of less than 20% (16.9%, about 18%, and 7.8%, respectively), while deep fractionation yielded a roughly 2-times higher median CV of about 34%.

The dynamic range of each workflow was determined by mapping the plasma protein data to known human plasma concentrations. The results of this analysis are summarized in FIG. 5D, which shows that five particle panel workflow covers a greater dynamic range and a greater number of low concentration proteins than the other three workflows.

FIG. 5E provides the proteomic data from each workflow in terms of plasma proteome coverage, showing the percent coverage at each intensity range, ranking proteins from high- to low-abundance. While high-pH fractionation covers 18% more high-abundance proteins (defined as top 50% intensity) than the five particle panel workflow, the five particle panel workflow provides up to 10-times higher coverage across the lowest 2 orders of magnitude, capturing 62% more proteins at the lower 50% intensity levels, comparatively.

The overlap between identified protein groups across the four workflows is shown in FIG. 5F. Out of the 1706 identified protein groups across the three assay replicates for the five particle panel workflow, 900 are uniquely identified, 184 of them are commonly identified in all four workflows, and 169 of them are common only to the high-pH fractionation. Comparatively, the high-pH fractionation workflow only provides 172 unique protein groups.

FIG. 5G compares the protein group identifications between the five particle panel and high-pH depletion workflows based on their functional annotations (phosphoproteins, signal transduction proteins, protein complexes, transport proteins, immune system process-related proteins, secreted protein, and lipoproteins). Compared to high-pH fractionation, the five particle panel workflow covers 2- to 9-times as many protein groups for the seven categories queried.

### Example 4

### Comparison of Particle Panel Enrichment to Plasma Depletion for Protein Identification with Data-Dependent Mass Spectrometry

This example compares protein group identifications with data-dependent mass spectrometric analysis using the five particle panel, high-pH fractionation (“Deep Fractionation”), and neat plasma workflows outlined in Example 3. Separate analyses were performed with two separate Deep Fractionation samples (‘Deep Fract.-In’ and ‘Deep Fract.-out’).

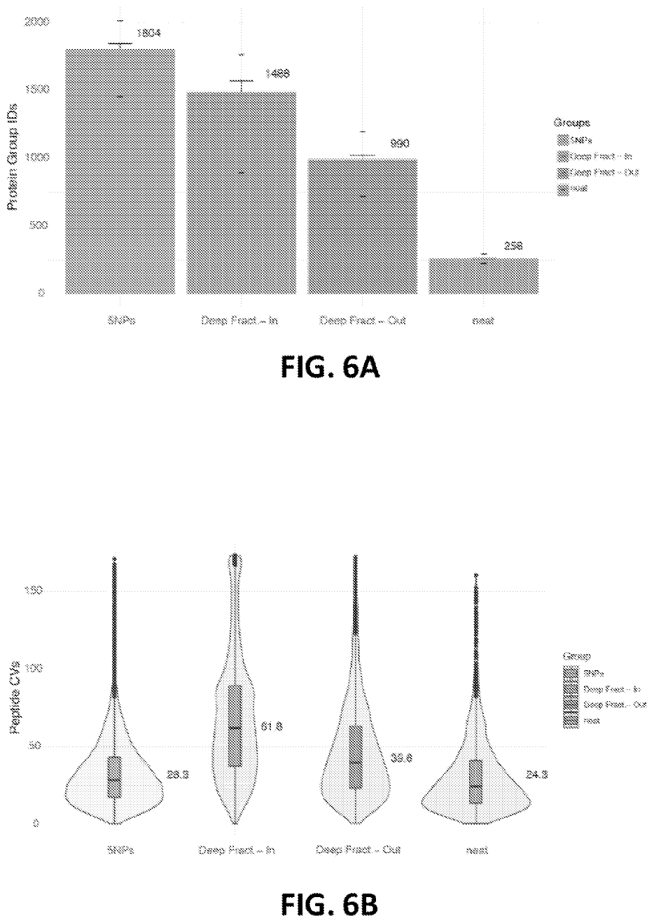

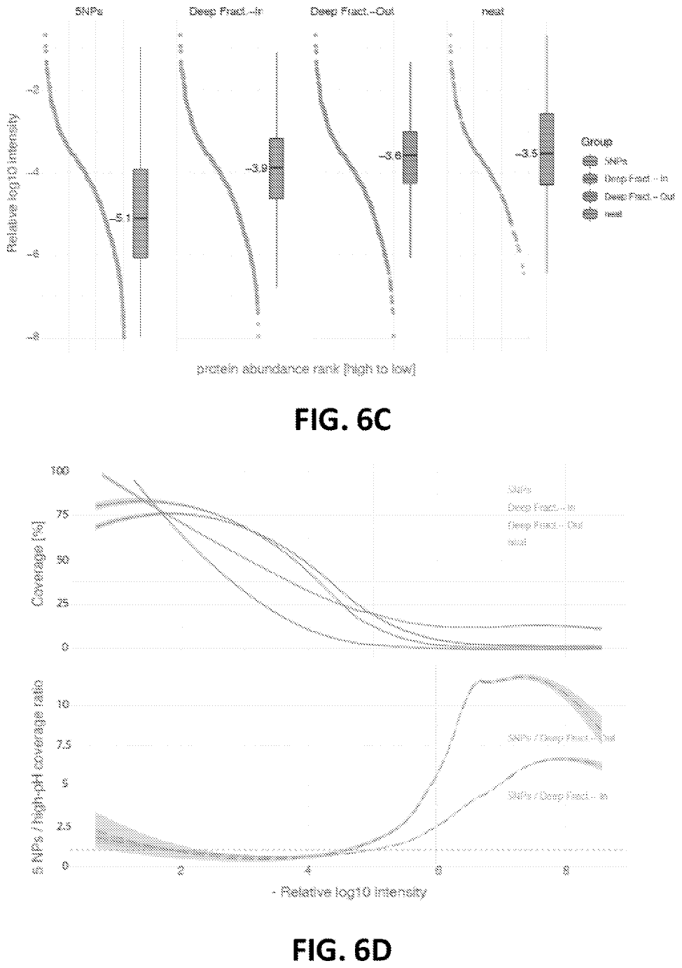

FIG. 6A provides the median number of protein groups identified for each workflow. Error bars denote standard deviations of assay replicates. The top dash depicts the number of identified proteins in any of the samples and the lower dash represents the number of identified proteins in 3 out of 3 assay replicates (complete features). The five particle panel workflow identified about 300 more proteins than the first high-pH fractionation sample, nearly double the number of proteins identified by the second high-pH fractionation sample, and about 7-times more proteins than the neat plasma workflow. FIG. 6B provides coefficients of variation (CV) of median normalized peptide intensities filtered for identifications across assay replicates, with median CV depicted on each plot.

FIG. 6C provides the dynamic range of identified proteins identified with each workflow, with the median log intensity of complete features is shown on each boxplot and the outliers removed.

FIG. 6D provides percent coverage of the human proteome in each workflow (top) and a comparison of the relative coverage of the human proteome by the five particle panel workflow and the first high-pH fractionation sample (bottom) over negative relative protein log 10 intensities. 95% interval is shown in grey.

### Example 5

### Plasma Proteome Interrogation by a 10-Particle Panel

This example covers plasma proteome analysis with a panel of 10 distinct particles differing in physicochemical properties. Plasma preparation, particle-based protein collection, and mass spectrometric analysis were performed as outlined in Examples 1 and 2. The proteomic data acquired with these particles, along with proteomic data obtained from neat and from depleted plasma, were analyzed in terms of number of identifications, precision, and quantitative and qualitative differences in the protein corona compositions.

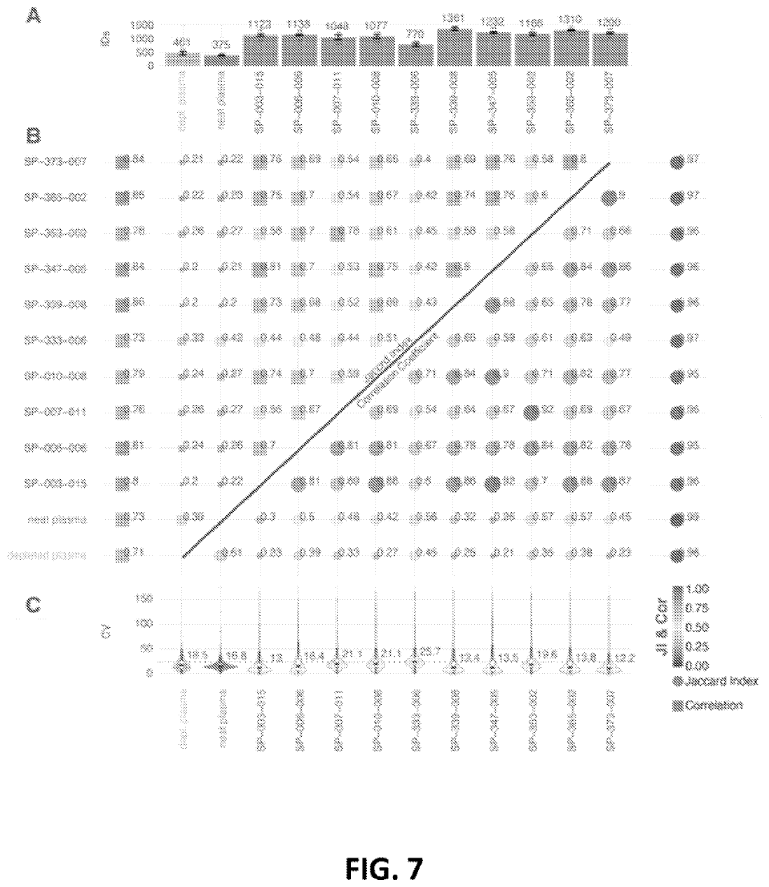

FIG. 7 panel A provides the median number of protein groups identified from neat and depleted plasma and the ten particles. Error bars denote standard deviations of protein IDs in assay replicates. The lower dash represents the number of identified proteins across assay replicates (complete features). For depleted and neat plasma median count and standard deviation across three assay replicates are shown as bar plots. The top dash depicts the number of proteins identified in any of the samples

To determine to what degree proteins identified across NPs and compared to plasma overlap, the Jaccard Index (“JI”) was calculated for each pair of particles and between the particles and neat and depleted plasma analyses. The upper triangle of FIG. 7 panel B provides JI indicating degree of overlapping identifications for each pair of particles and for neat and depleted plasma. Each box size is scaled by the magnitude of JI. The lower triangle of FIG. 7 panel B provides Pearson Correlation Coefficients (“r”) indicating correlation of median normalized log 10 intensities as the mean r across assay replicates (right column) and comparing individual NPs and neat plasma Circle size is scaled by the magnitude of r. The qualitative reproducibility of each assay triplicate is provided in FIG. 7 panel C, which provides the mean correlation coefficient (close to 1) and the coefficient of variation for each particle and for neat and depleted plasma.

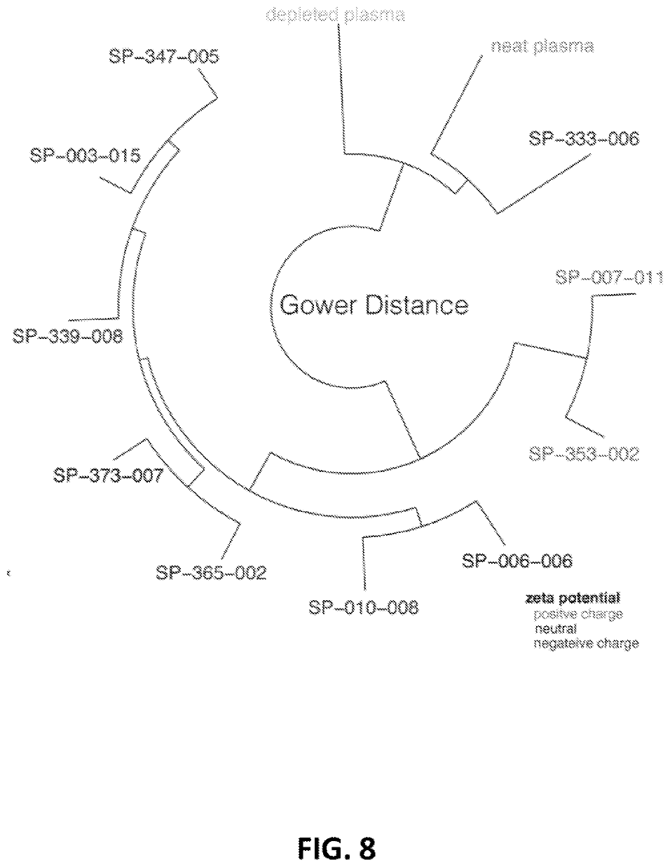

To further map out similarity and dissimilarity across all particles as well as neat and depleted plasma, the Gower distance was calculated for each pair of particles and between each particle and the neat and depleted plasma samples, based on their proteomic profiles. FIG. 8 summarizes the results as a distance tree for median protein intensities. As can be seen from this chart, the clustering primarily coincides with negative and positive zeta potential. However, some particles that cluster together (like SP-365 and SP-373) do not strictly follow that rule (FIG. 2E), suggesting that protein abundance signatures on particles are driven by more complex aspects of their physicochemical properties.

### Example 6

### Comparison of Particle Properties with Protein Corona Composition for a Ten Particle Panel

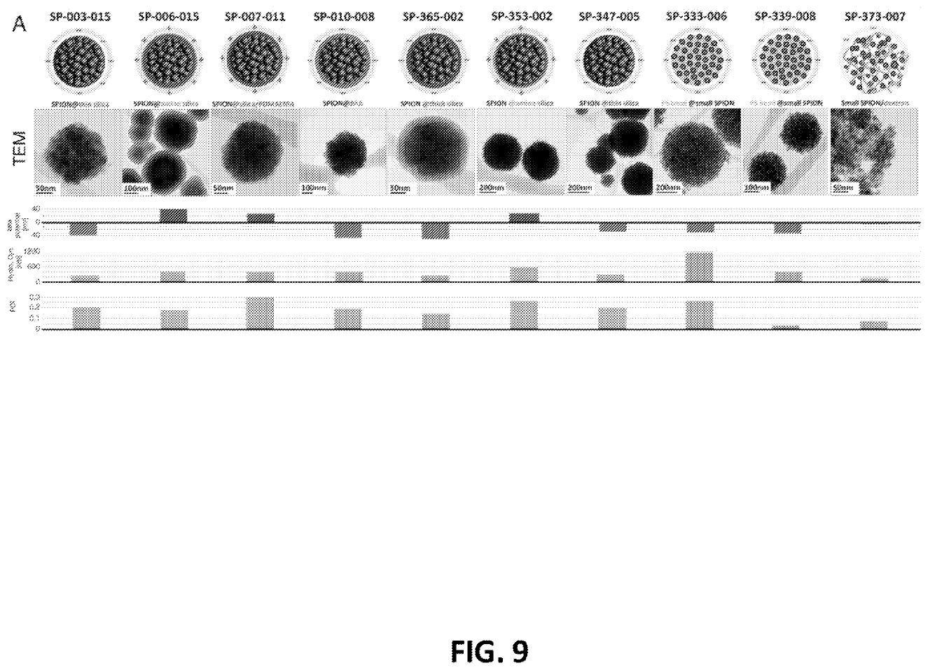

In order to explore the degree to which protein corona composition can be explained based on particle physicochemical properties, the physicochemical properties and protein corona compositions were compared for 10 different particles. Plasma protein analysis was performed according to the methods outlined in Examples 1 and 2. Particle characterization was performed with transmission electron microscopy (TEM) and dynamic light scattering (DLS). TEM was used to assess the morphology and surface features of the particles. DLS measurements were performed to determine the particle characteristics in the solution, such as hydrodynamic size and polydispersity index (PDI). These measurements show that the 10 particle panel includes a variety of features, with sizes ranging from −150 nm to 1 μm. With the exception of SP-373-007, which appears to be a mixture of dextran networks with embedded small NPs with sizes of −10-15 nm (depicted in FIG. 9 panel A), the TEM indicated that all NP samples have spherical or semi-spherical morphologies. FIG. 9 panel A provides TEM images of each particle, along with their zeta potential, hydrodynamic radii, and PDI (bar graphs below images.

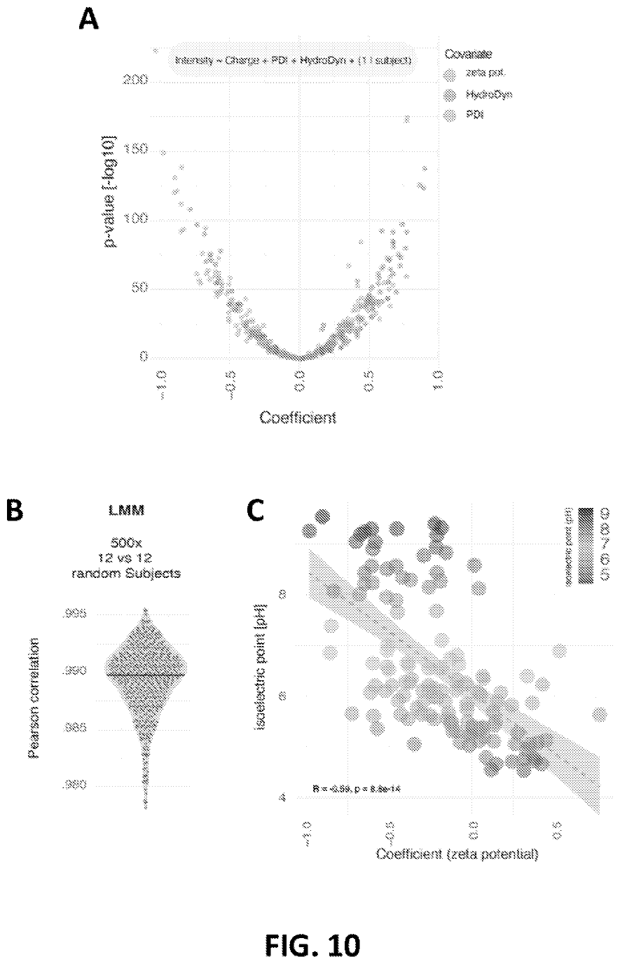

FIG. 10 panel A provides a volcano plot depicting the coefficients derived from the a model for protein binding based on the properties of three specific particles, with each protein plotted against the p-value. FIG. 10 panel B provides results from 500 random samplings selecting 2× 12 non-overlapping subjects assayed with the 10 particles to build a linear mixed effects model for which the Pearson correlation was calculated. FIG. 10 panel C provides correlation coefficients between the determined coefficients for each protein and the zeta potential of the particle. Color indicates the predicted isoelectric point for each protein. The gray shaded regression line is depicted with 95% confidence interval.

### Example 7

### Robust, High Throughput and Deep Plasma Proteomics Workflow with Engineered Nanoparticle Panels

Data Independent Acquisition (DIA) proteomics may be used to catalogue thousands of proteins in complex biological samples like human blood plasma in a high throughput LC-MS proteomics approach. For large-scale proteomics studies, it is advantageous for LC and MS systems to be robust to be widely available to users having various levels of analytical expertise, without compromising on peptide and protein coverage. The present disclosure describes a high throughput label-free plasma proteomics workflow utilizing Orbitrap Exploris 480 mass spectrometer coupled to UltiMate3000 nanoLC system and micro-pillar Array Columns (μPACTM). Results are obtained through a robust and high throughput analytical setup for in-depth proteomics analysis of plasma samples.

LC-MS analysis was performed with 50 cm and 110 cm, C18 μPAC columns (PharmaFluidics, Belgium) coupled to a Orbitrap Exploris 480 mass spectrometer (Thermo Fisher Scientific). Neat plasma, and five-nanoparticle (5 NPs) enriched plasma proteins were digested then analyzed with label-free MS data acquisition in DIA in top speed mode. Performance across different columns were evaluated with a 120 min single injection method or five separate injections with a 30 min DIA Method for each of the 5 NPs and the neat plasma digest, for comparing between the methods and assessing the improvement in throughput and protein depth of coverage with the NP based method. Proteome Discoverer™ 3.0 software and MaxQuant software were used for label-free data analysis providing improved peptide and protein coverage with a 1% FDR rate.

Label-free proteomics performance on the Orbitrap Exploris 480 MS was evaluated with 500 ng neat plasma and 5 NPs enriched protein digests in a 30 min reversed phase (RP) gradient on 50 cm, C18 μPAC column at 1 uL/min flow rate in a data independent acquisition method which resulted in about 1500 protein group identifications in a single pooled plasma digest as a baseline of performance. The 110 cm C18 μPAC was used with single injection 120 min gradient at 0.5 uL/min flowrate, injecting the 2.5 ug pooled 5 NP plasma digest. The pillar support structure allows LC flow with minimal back-pressure and allows steps involving sample load and column re-equilibration and end of gradient cleaning process. In some cases, the column re-equilibration and end of gradient cleaning process may affect the throughput of a nanoLC method done at higher flow rate of up to 1 μL. The injection was followed up with analytical separation of peptides with a lower nanoflow rate of 500 nL/min.

Proteome Discoverer 3.0 software, with an improved peptide identification workflow using a super-fast single step MSPepSearch against the human NIST Orbitrap HCD library and CHIMERYS provides the depth of coverage required for in-depth plasma proteomics workflow. Percolator FDR calculation is used to only allow those spectra within 1% FDR rate to be reported. We routinely identified at least 1500 protein groups from 0.5 μg of 5 NP enriched protein digest in less than 2.5 hours using this optimized workflow. The workflow may be further optimized to perform the method in less time, for instance, in less than 2 hours.

### Example 8

### Robust In-Depth Label-Free Plasma Proteomics with Engineered Nanoparticle Panels: An Evaluation of Micro-Pillar Array Columns and FAIMS Peptide Separation

LC-MS based proteomics analysis can be used for the identification and quantification of thousands of proteins in complex biological samples such as human blood plasma. For large-scale proteomics studies, it is advantageous for LC and MS systems to be robust to be widely available to users having various levels of analytical expertise, without compromising on peptide and protein coverage. The present disclosure describes a label-free plasma proteomics workflow on a Orbitrap Fusion Lumos Tribrid mass spectrometer coupled to a High-Field Asymmetric Waveform Ion Mobility Spectrometry (FAIMS) interface and micro Pillar Array Columns (μPACTM), as a robust analytical setup for in-depth proteomics analysis of plasma.

LC-MS analysis was performed with 110 and 200 cm C18 μPAC columns (PharmaFluidics, Belgium) coupled to a Orbitrap Fusion Lumos Tribrid mass spectrometer and FAIMS Pro Interface (Thermo Fisher Scientific). Neat plasma, five-nanoparticle (5 NP) enriched and digested plasma proteins, and standard HeLa digest (Pierce) were analyzed with label-free MS data acquisition in DDA, top speed mode with multi-CV FAIMS peptide fractionation based on peptide mass and charge states. Performance across different columns were evaluated with a 300 min single injection method. Proteome Discoverer™ 3.0 software and MaxQuant software were used for label-free data analysis providing improved peptide and protein coverage with a 1% FDR rate.

Label-free proteomics performance on the Orbitrap Fusion Lumos Tribrid MS was evaluated with 4 ug of standard HeLa digest in a 300 min reversed phase (RP) gradient on 200 cm, C18 μPAC columns in a data dependent acquisition method with FAIMS Pro Interface. The method resulted in at least 10,000 proteins and at least 120,000 peptide identifications. The μPAC performance allows loading up to 4 μg sample on column with nanoflow sensitivity concurrent with the robustness of an analytical column. The pillar support structure allows LC flow with minimal back-pressure, which allows steps involving sample load and column re-equilibration and end of gradient cleaning process. Column re-equilibration and end of gradient cleaning process sometimes may affect the throughput of a nanoLC method done at higher flow rate of up to 1 μL followed by analytical separation of peptides with lower nanoflow rate of 600 nL/min.

Proteome Discoverer 3.0 software, with an improved peptide identification workflow using a super-fast single step MSPepSearch against the human NIST Orbitrap HCD library and CHIMERYS provides the depth of coverage required for in-depth plasma proteomics workflow. Percolator FDR calculation was used to only allow those spectra within 1% FDR rate to be reported. We routinely identified about 9500 proteins and about 120,000 peptide groups from 4 μg of bulk HeLa digest in about 5 hours using this optimized workflow. The workflow may be performed with a single injection for deep plasma proteomics analysis.

### Example 9

### Modeling Nano-Bio Interactions of 37 Engineered Nanoparticles that Enable Deep Plasma Proteomics Studies at Unprecedented Scale

Introducing a nanoparticle (NP) into a biofluid such as blood plasma leads to the formation of a selective, specific, and reproducible protein corona at the nano-bio interface driven by the relationship between protein-NP affinity, protein abundance and protein-protein interactions. There are numerous NP physicochemical design possibilities which can be tailored to enhance and differentiate protein selectivity. This example illustrates the relationship between nanoparticle chemical functionalization and corona formation building linear mixed effects models that may enhance NP design.

A set of 37 engineered nanoparticles with specific physicochemical properties were processed with Proteograph interrogating a pooled plasma sample. Proteomics data were acquired using 30-minute LC runs with an Orbitrap Lumos. MaxQuant raw data processing identified more than 1,500 protein groups at 1% protein and peptide FDR. By developing machine learning (linear mixed-effects) models, identified significant relationships between physicochemical NP properties (including zeta potential, amine, and carboxy functionalization) and differential abundance of individual proteins and protein classes within NP corona were identified. For example, 23% of the abundance of C-reactive protein (CRP) in a protein corona was associated with NP zeta potential, and 22% could be allocated to polymeric and sugar surface functionalization. In contrast, the abundance of plasma kallikrein (KLKB1) was observed to be unaffected by NP zeta potential but more than 50% driven by sugar functionalization.

The results suggest that the relationship between NP surface functionalization and specific proteins or protein classes in complex biological samples may be modeled. This information may guide NP design to further increase the utility of the Proteograph platform in proteomics research and biomarker discovery.

### Example 10

### Protein Depletion and Peptide Fractionation Improve Proteome Depth of Coverage when Coupled with Nano-Particle Corona Formation

This example demonstrates a significant improvement of NP-based biomolecule assay performance in terms of proteome coverage by depleting the input plasma prior to biomolecule corona formation on a particle.

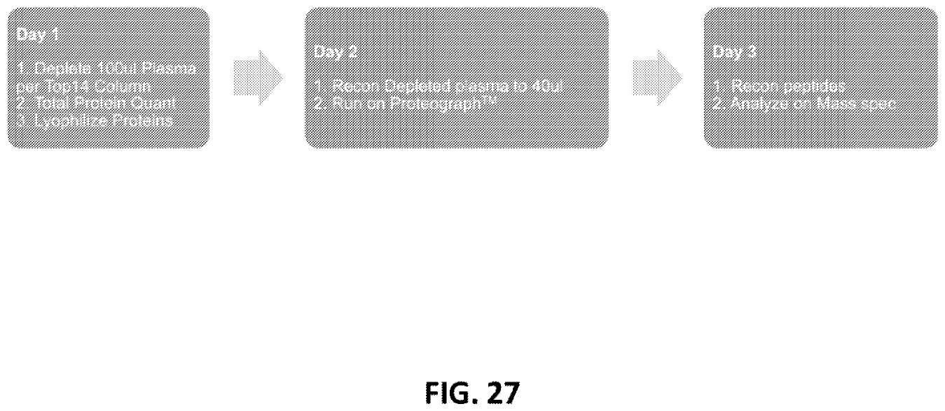

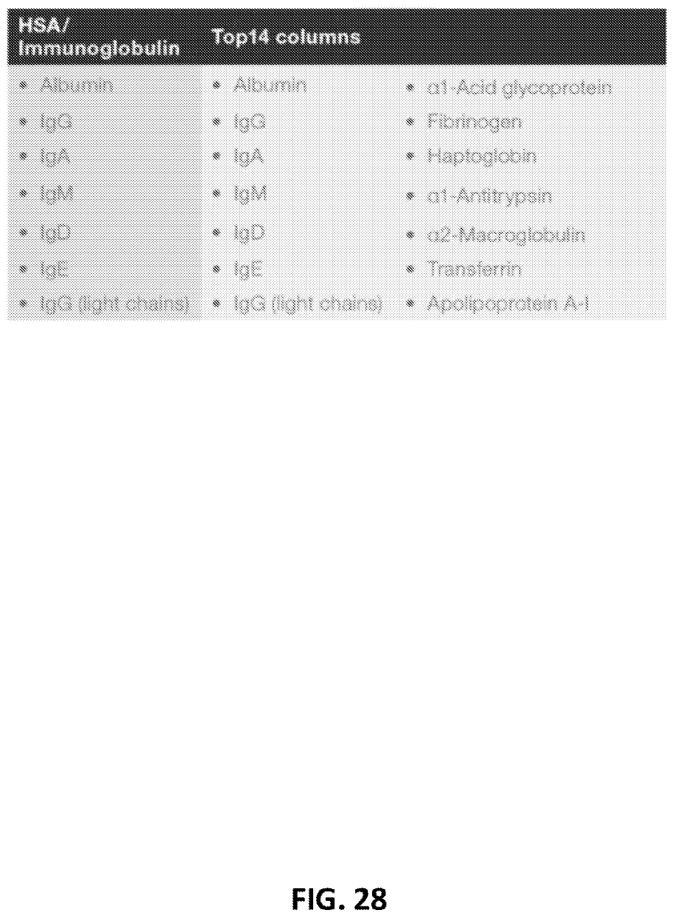

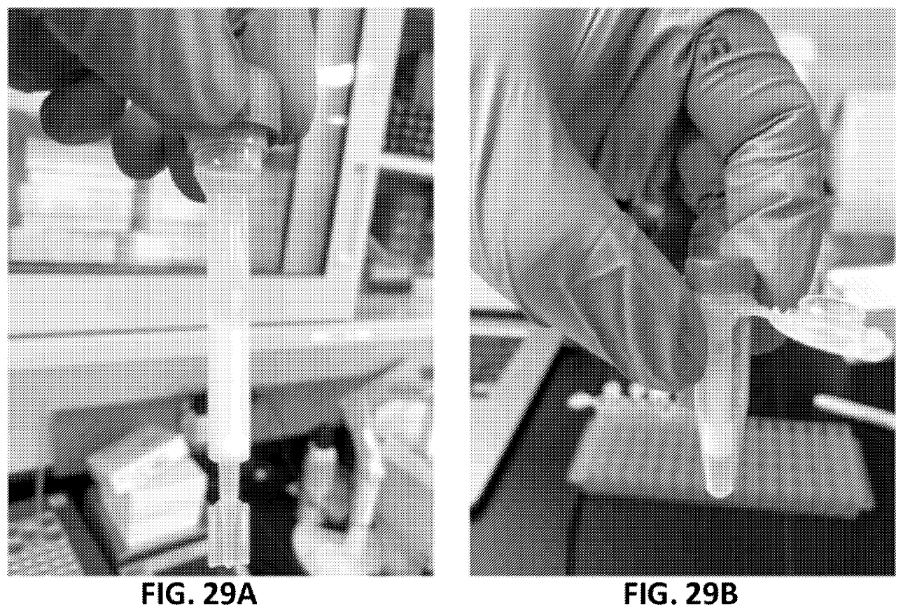

An example of the workflow is shown in FIG. 27. A plasma volume of 200 ul was depleted in Top14 resin spin columns (100 ul per spin column). The depleted sample was lyophilized and then reconstituted to 40 ul. The reconstituted sample was then contacted with V1.2 particles to enrich the sample. The enriched sample from the particles were separated from the V1.2 particles and then reconstituted, and then they were injected into a mass spectrometer. For comparison, in different arms of the study, plasma was co-incubated with Top14 resin and V1.2 particles (40 ul and 100 ul), or plasma was incubated only V1.2 particles, or plasma was incubated only with depletion resin.

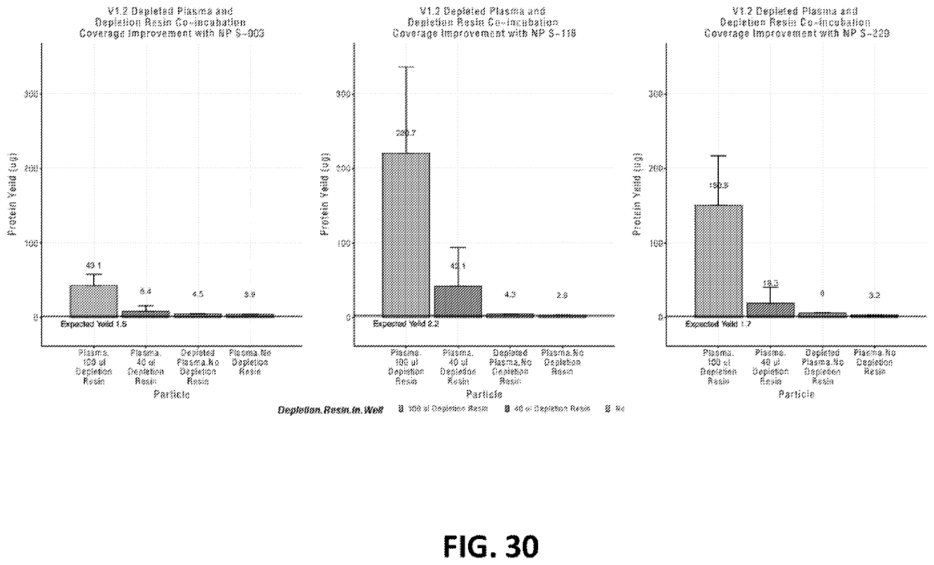

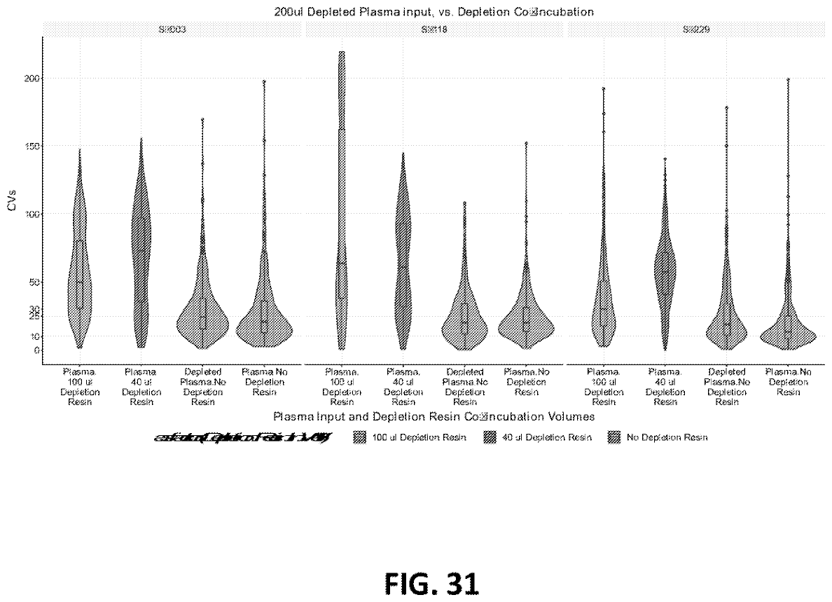

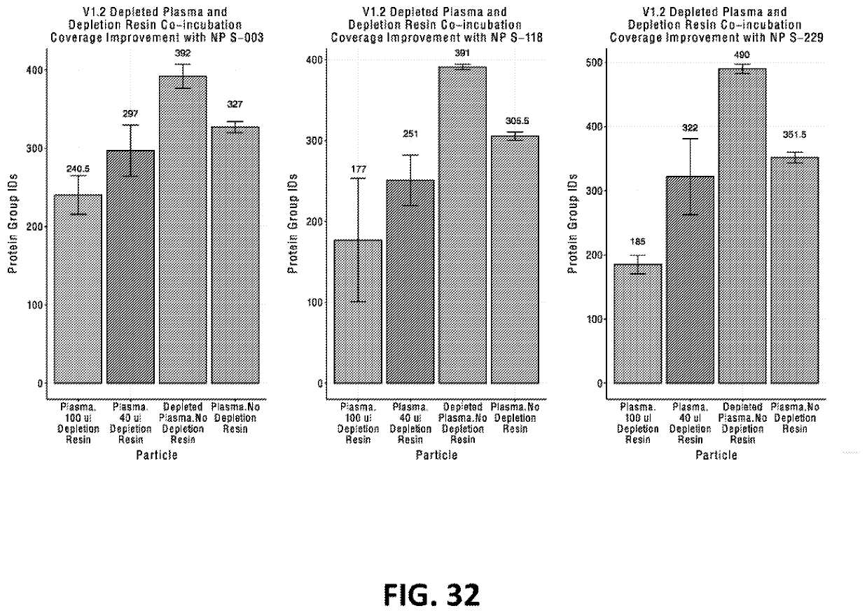

The total amount of protein quantified from the experiments are shown in FIG. 30. Proteome coverage from the experiments are shown in FIG. 30. Depleting plasma before enriching with V1.2 particles increased proteome coverage by 20-30%.

### Numbered Embodiments

Embodiments contemplated herein include embodiments 1 to 20.

Embodiment 1. An apparatus for assaying a biological sample, comprising: (a) one or more transfer units; (b) a sample storage unit configured to receive and retain the biological sample, wherein the sample storage unit is operably coupled to the one or more transfer units; (c) a partition containing therein a particle, wherein the partition is operably coupled to the one or more transfer units; (d) a plurality of columns comprising a trap column, a depletion column, and an analytical column, wherein the plurality of columns is in fluid communication with each other and operably coupled to the one or more transfer units; and (e) a control unit comprising one or more processors, wherein the control unit is in electrical communication with the one or more transfer units.

Embodiment 2. The apparatus of embodiment 1, wherein the one or more transfer units is configured to transfer the biological sample from the sample storage unit to the depletion column to produce a depleted sample.

Embodiment 3. The apparatus of embodiment 2, wherein the one or more transfer units is configured to transfer the depleted sample to the partition to adsorb a plurality of biomolecules from the biological sample onto the particle.

Embodiment 4. The apparatus of embodiment 3, wherein the one or more transfer units is configured to transfer the plurality of biomolecules to the trap column to produce a purified sample.

Embodiment 5. The apparatus of embodiment 4, wherein the one or more transfer units is configured to transfer the purified sample to the analytical column to produce a separated sample.

Embodiment 6. The apparatus of embodiment 5, wherein the one or more transfer units is configured to transfer the separated sample to a mass spectrometer for mass spectrometry analysis on the plurality of biomolecules to produce a plurality of signals.

Embodiment 7. The apparatus of embodiment 1, further comprising a plurality of reagent storage units comprising a first reagent storage unit containing therein an aqueous solvent and a second reagent storage unit containing therein an elution solvent, wherein the one or more transfer units is operably coupled to the plurality of reagent storage units.

Embodiment 8. The apparatus of embodiment 1, wherein the one or more transfer units comprises a pipette.

Embodiment 9. The apparatus of embodiment 1, wherein the one or more transfer units comprises a fluidic connection.

Embodiment 10. The apparatus of embodiment 1, wherein the sample storage unit is configured to receive and retain a plurality of biological samples.

Embodiment 11. The apparatus of embodiment 1, further comprising a sample preparation unit comprising at least one of an HPLC column, a filter, and a centrifuge, wherein the one or more transfer units is operably coupled to the sample preparation unit.

Embodiment 12. The apparatus of embodiment 1, further comprising a second partition containing therein a second particle.

Embodiment 13. The apparatus of embodiment 1, wherein the particle is a paramagnetic particle.

Embodiment 14. The apparatus of embodiment 1, wherein the partition contains therein at least two different particles types.

Embodiment 15. The apparatus of embodiment 1, wherein the one or more transfer units is configured to transfer the biological sample from the sample storage unit to adsorb a plurality of biomolecules from the biological sample onto the particle.

Embodiment 16. The apparatus of embodiment 15, wherein the one or more transfer units is configured to transfer the plurality of biomolecules to the trap column to produce a purified sample.

Embodiment 17. The apparatus of embodiment 16, wherein the one or more transfer units is configured to transfer the purified sample to the depletion column to produce a depleted sample.

Embodiment 18. The apparatus of embodiment 17, wherein the one or more transfer units is configured to transfer the depleted sample to the analytical column to produce a separated sample.

Embodiment 19. The apparatus of embodiment 18, wherein the one or more transfer units is configured to transfer the separated sample to a mass spectrometer for mass spectrometry analysis on the plurality of biomolecules to produce a plurality of signals.

Embodiment 20. The apparatus of embodiment 1, wherein the analytical column comprises a plurality of micropillars disposed therein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of selecting surfaces for a biomolecule assay, comprising:
(a) providing one or more biological samples comprising a plurality of biomolecules;
(b) contacting the one or more biological samples with a plurality of surfaces, such that each surface in the plurality of surfaces adsorbs a subset of biomolecules in the plurality of biomolecules;
(c) determining, for each surface in the plurality of surfaces, abundances of the subset of biomolecules adsorbed thereon; and
(d) selecting a subset of surfaces in the plurality of surfaces based at least in part on the abundances when the subset of surfaces adsorbs biomolecules or biomolecule groups that comprise a different abundance pattern compared to another subset of surfaces in the plurality of surfaces.

2. The method of claim 1, wherein the subset of surfaces is selected when a first surface in the subset of surfaces binds a first set of functionally-related and/or structurally-related biomolecules.

3. The method of claim 2, wherein the subset of surfaces is selected when a second surface in the subset of surfaces binds a second set of functionally-related and/or structurally-related biomolecules.

4. The method of claim 2 or 3, wherein the first set, the second set, or both functionally related biomolecules comprises at least one of: a hormonal protein, a cytolytic protein, an innate immunity protein, a membrane attack complex, a complement pathway protein, an amyloid fibril, a protein involved in cholesterol metabolism, a protein involved in steroid metabolism, a protein with gamma carboxyglutamic acid domains, a protein associated with amyloidosis, a sulfated protein, a proteoglycan protein, an immunoglobulin, an adaptive immunity protein, a mitochondrial protein, a membrane protein, a cell shape protein, a muscular protein, a protein that binds to genetic material, a protein associated with gene expression and/or regulation, a protein associated with intra and/or extracellular space, and any combination thereof.

5. The method of any one of claims 2-4, further comprising contacting a new biological sample, not among the one or more biological samples, with the subset of surfaces to thereby assay the first set or the second set of functionally-related and/or structurally-related biomolecules in the new biological sample.

6. The method of any one of claims 2-5, wherein the first surface and the second surface each adsorbs a given biomolecule in the plurality of biomolecules at a different relative abundance.

7. The method of any one of claims 2-6, wherein the first surface adsorbs at least one biomolecule that is not adsorbed on the second surface.

8. The method of any one of claims 1-7, wherein the one or more biological samples are samples obtained from subjects afflicted with a given disease, such that the selected subset of surfaces is optimized for assaying a new biological sample for the given disease.

9. The method of claim 8, further comprising contacting a new biological sample, not among the one or more biological samples, with the subset of surfaces to thereby probe biomolecules in the new biological sample for determining a disease state of the new biological sample related to the given disease.

10. The method of any one of claims 1-7, wherein the one or more biological samples are obtained from an individual, such that the selected subset of surfaces is optimized for assaying biological samples from the individual.

11. The method of any one of claims 1-7, wherein the one or more biological samples are obtained from a group of individuals having at least one attribute, such that the selected subset of surfaces is optimized for assaying biological samples from individuals having the at least one attribute.

12. The method of claim 11, wherein the at least one attribute comprises a genetic factor, a non-genetic factor, or both.

13. The method of claim 12, wherein the genetic factor comprises one or more genetic mutations, presence or absence of one or more alleles, presence or absence of one or more genes, presence or absence of one or more chromosomes, or any combination thereof.

14. The method of claim 12 or 13, wherein the non-genetic factor comprises a level of physical activity, quality and pattern of sleep, consumption of drugs and/or alcohol, biometrics, or any combination thereof.

15. The method of any one of claims 1-7, wherein the one or more biological samples are samples obtained from one or more species, such that the selected subset of surfaces is optimized for assaying for the at least one species in the one or more species.

16. The method of any one of claims 3-15, wherein the first surface and the second surface is chosen when a Jaccard index between the identities of the distinct subset of biomolecules adsorbed on the first surface and the second surface is at most about 0.1, 0.2, 0.3, 0.4, 0.5, 0.6, 0.7, 0.8, 0.9.

17. The method of any one of claims 3-16, wherein the first surface and the second surface is chosen when a Pearson correlation index between measured intensities of the first set of functionally-related biomolecules and the second set of functionally-related biomolecules is at most about 0.1, 0.2, 0.3, 0.4, 0.5, 0.6, 0.7, 0.8, 0.9, or 1.

18. The method of any one of claims 1-17, wherein the subset of surfaces is selected when the subset of surfaces adsorbs biomolecule or biomolecule groups at a greater dynamic range compared to the another subset of surfaces in the plurality of surfaces.

19. The method of claim 18, wherein the greater dynamic range is at least about 1, 2, 3, 4, 5, 6, 7, 8, 9, or 10 magnitudes greater.

20. The method of any one of claims 1-19, wherein the one or more biological samples comprises derivatives or portions of the same given biological sample.

21. The method of any one of claims 1-19, wherein the one or more biological samples comprises human blood plasma samples.

22. The method of any one of claims 1-19, wherein the one or more biological samples comprises a biological sample standard.

23. The method of claim 22, wherein the biological sample standard is a HeLa cell extract.

24. The method of any one of claims 1-23, wherein the plurality of biomolecules comprises polyamino acids.

25. The method of claim 24, wherein the polyamino acids comprise peptides, proteins, or a combination thereof.

26. The method of any one of claims 1-25, wherein the distinct subset of biomolecules adsorbed on at least one surface in the plurality of surfaces comprises at least two biomolecules that do not share a common binding motif.

27. The method of any one of claims 1-26, wherein the determining the identities in (c) is performed by: (i) desorbing the distinct subset of biomolecules adsorbed on each surface in the plurality of surfaces to produce desorbed biomolecules, (ii) performing mass spectrometry on the desorbed biomolecules to produce mass spectrometry signals, and (iii) quantifying the mass spectrometry signals to determine the identities of the distinct subset of biomolecules.

28. The method of claim 27, wherein (i) further comprises digesting at least a portion of the distinct subset of biomolecules to produce desorbed biomolecules.

29. The method of claim 28, wherein the digesting comprises contacting the distinct subset of biomolecules with a protease.

30. The method of any one of claims 1-29, wherein each surface in the plurality of surfaces adsorbs a distinct subset of biomolecules in the plurality of biomolecules.

31. The method of claim 30, wherein a first distinct subset of biomolecules adsorbed on a first surface in the plurality of surfaces comprises at least one common biomolecule with a second subset of biomolecules adsorbed on a second surface in the plurality of surfaces.

32. The method of claim 30 or 31, wherein the first distinct subset of biomolecules and the second subset of biomolecules comprises at least one biomolecule not in common.

33. The method of any one of claims 1-32, wherein the different abundance pattern comprises enrichment of low abundance biomolecules relative to the plurality of biomolecules in the one or more biological samples.

34. A method of producing an enriched biological sample, comprising:
(a) providing a sample comprising a plurality of biomolecules;
(b) contacting the sample with a particle or resin to specifically bind at least one biomolecule or biomolecule class target in the sample to the particle or resin;
(c) separating the particle or resin and the at least one biomolecule from the sample, thereby producing a depleted sample;
(d) contacting the depleted sample with a surface, wherein the surface is configured to adsorb a set of biomolecules in the depleted sample on the surface;
(e) separating the set of biomolecules and the surface from the depleted sample; and
releasing the set of biomolecules from the surface to produce an enriched sample comprising the set of biomolecules.

35. The method of claim 34, wherein the at least one biomolecule or biomolecule class target comprises: albumin, IgG, IgA, IgM, IgD, IgE, IgG (light chain), alpha-1-acid glycoprotein, fibrinogen, haptoglobin, alpha-1-antitrypsin, alpha-2-macroglobulin, transferrin, apolipoprotein A-1, or any combination thereof.

36. The method of claim 34 or 35, wherein the at least one biomolecule or biomolecule class target comprises a predetermined subset of the plurality of biomolecules comprising a high relative abundance.

37. The method of any one of claims 34-36, wherein in step (c), the separating reduces an abundance of the at least one biomolecule or biomolecule class target at least by a factor of 2, 5, 10, or 100.

38. The method of any one of claims 35-37, wherein in step (c), producing the depleted sample yields at least about 30% more unique proteins, protein groups, or peptides in the enriched sample of step (f).

39. The method of any one of claims 35-38, wherein in step (c), producing the depleted sample yields a larger dynamic range of at least about 1 magnitude in the unique proteins or protein groups in the enriched sample of step (f).

40. The method of any one of claims 35-39, further comprising after step (c) or before step (d), drying and reconstituting the depleted sample to a predetermined concentration or volume.

41. The method of claim 35-40, further comprising after step (e), drying and reconstituting the enriched sample to a predetermined concentration or volume.

42. The method of any one of claims 35-41, wherein the method is performed in less than about 72 hours.

43. The method of any one of claims 35-42, wherein the biomolecule comprises a protein or protein group.

44. The method of any one of claims 35-43, wherein the surface is a nanoparticle surface.

45. The method of any one of claims 35-44, further comprising contacting the depleted sample with a second surface, wherein the second surface is configured to adsorb a second set of biomolecules in the depleted sample on the second surface.

46. The method of any one of claims 35-45, wherein the releasing in (f) further comprises digesting the set of biomolecules.

47. The method of any one of claims 35-46, wherein the particle or resin is disposed in a column.

48. A kit for enriching a biological sample, comprising:
a first substance configured to specifically bind to a first set of biomolecule targets;
a second substance configured to adsorb a second set of biomolecule targets; and
a third substance configured to adsorb a third set of biomolecule targets.

49. The kit of claim 48, wherein the first substance is a resin or a particle.

50. The kit of claim 48 or 49, wherein the first substance comprises a specific binding moiety configured to bind to the first set of biomolecule targets.

51. The kit of any one of claims 48-50, wherein the first substance is configured to specifically bind to at least one of: albumin, IgG, IgA, IgM, IgD, IgE, IgG (light chains), alpha-1-acid glycoprotein, fibrinogen, haptoglobin, alpha-1-antitrypsin, alpha-2-macroglobulin, transferrin, and apolipoprotein A-1.

52. The kit of any one of claims 48-51, further comprising a fourth substance configured to non-specifically bind to a fourth set of biomolecule targets.

53. The kit of any one of claims 48-52, further comprising a fifth substance configured to non-specifically bind to a fifth set of biomolecule targets.

54. The kit of any one of claims 48-53, wherein the second substance comprises a plurality of domains, wherein each domain in the plurality of domains is configured to non-specifically bind to a distinct subset in the second set of biomolecule targets.

55. The kit of any one of claims 48-54, wherein the second substance comprises a particle surface, and the plurality of domains comprises a plurality of surface regions on the particle surface.

56. The kit of any one of claims 48-55, wherein the second substance comprises a plurality of particle surfaces, and the plurality of particle surfaces are disposed on a plurality of particles.

57. The kit of any one of claims 48-56, wherein the kit comprises a chamber or a well having the first substance, the second substance, and the third substance disposed therein.

58. The kit of claim 48, wherein the chamber comprises a column.

59. The kit of claim 48, wherein the chamber comprises a microfluidic channel.

60. The kit of claim 48, wherein a surface region of the well comprises the first substance.
